(19) United States
(12) Patent Application Publication
(10) Pub. No.: US 2009/0022699 A1
(43) Pub. Date: Jan. 22, 2009

(54) Method Of Genotypically Modifying Cells By Administration Of RNA

(75) Inventors: Stephen Ray, Bath (GB); Michael Fischer, Berkshire (GB)

Correspondence Address: WOODCOCK WASHBURN LLP, CIRA CENTRE, 12TH FLOOR, 2929 ARCH STREET, PHILADELPHIA, PA, 19104-2891, US

(73) Assignee: FISCHER EDUCATION PROJECT LIMITED, Oxfordshire (GB)

(21) Appl. No.: 11/814,271

(22) PCT Filed: Jan. 19, 2006
(86) PCT No.: PCT/GB2006/000184

§ 371 Date: Jun. 6, 2008

(30) Foreign Application Priority Data
Jan. 19, 2005 (GB) 0501125.9

(51) Int. Cl. A61K 48/00; C12N 15/87; C12N 5/10; A61K 35/12; A61P 35/00; A61P 7/00; A61K 31/7105

(52) U.S. Cl. 424/93.21; 435/440; 435/455; 435/468; 435/325; 435/419

(57) ABSTRACT

A method of inducing genotypic modification in a cell, which comprises providing isolated RNA comprising RNA extractable from source tissue to the cell under conditions whereby the desired induction of genotypic modification is achieved, wherein the RNA is isolated polyA positive RNA in substantially pure form.

### FIELD OF THE INVENTION

The present invention relates to the alteration of cell properties. In particular, it relates to the alteration of the genotype of a cell, both in vitro and in vivo. The invention further relates to the induction of differentiation of stem cells.

### BACKGROUND OF THE INVENTION

Genetic modification of cells can be achieved by treatment of the cells with DNA sequences. Typically, such methods are used to alter one or a few bases at a specific location within the target genome. For example, ‘Small Fragment Homologous Replacement’ (Goncz et al (2002) Gene Ther 9, 691-4) uses a sense DNA oligonucleotide of 200 to 500 bases encoding the desired sequence modification towards its centre. Similar methods have been described by Campbell et al (1989) New Biol 1, 223-7 and Igoucheva (2001) Gene Ther 8, 391-9. The oligonucleotides used in these methods are generally DNA or RNA:DNA hybrids. However, the transformation efficiencies for these in vitro methods are low, generally between 0.01 and 1%.

Accordingly, there is a need to provide further methods for the genetic modification of cells, both in vitro and in vivo. RNA extracts have been shown to affect the differentiation of tissues, for example when applied to chick embryos (Sanyal et al (1966) PNAS, 55:743-750), mouse ascite cells (Niu et al (1961) PNAS, 47:1689-1700) and mouse uteri (Yang et al (1977) PNAS 74:1894-1898). They have also been shown to affect the properties of neoplastic cells, for example in rat hepatoma cells (DeCarvalho et al (1961) Nature, 189:815-817) and leukaemic patients (DeCarvalho et al (1963) Nature 197:1077-1079). Moreover, RNA extracts have been shown to have effects in the immune system, for example in the transfer of immune properties from donor to recipient (Rascati et al (1981) Intervirology 15:87-96 and DeLuca et al (2001) Molecular and Cellular Biochemistry 228:9-14).

The present invention is based on the discovery that RNA extracted from donor cells may be used to induce genetic modification in target cells. The genetic characteristics induced in the target cells are unique to the donor cells and are inheritable at the cellular level. The transformation efficiency may be better than current methods used in the art, approaching 100% under preferred conditions. The present invention is also based on the discovery that when the target cells are stem cells, the RNA extracts may also induce differentiation.

### SUMMARY OF INVENTION

The present invention is concerned with the alteration of cell properties. In particular, it relates to the alteration of the genotype of cells. The invention is also concerned with the control of differentiation of stem cells.

Without wishing to be bound by theory, the present invention extends a hypothesis presented by the present inventors in co-pending international patent application PCT/GB20041002981 that alteration of the genotype of a cell may be effected by the transfer of information from one cell to another via RNA.

Like the invention disclosed in PCT/GB2004/002981, the present invention is concerned with the alteration of the genotype of a cell, and the treatment of various disease conditions by alteration of the genotype of cells. The present inventors have also found that it is possible to induce stem cells to differentiate into a desired differentiated cell type. This is achieved by providing specific RNA sequences to the target cells.

The ability to influence genotype and cell differentiation allows a variety of clinically useful phenomena to be induced including allowing the genetic constitution of cells to be altered, allowing specific cell types and cell fates to be induced, allowing immunological profiles to be changed at will, allowing the induction of particular immune functions and so on. The ability to induce genotypic alteration and stem cell differentiation in vivo means that stem cell-mediated functional repair may be beneficially promoted in intact organisms, and particularly animals.

PCT/GB2004/002981 describes a method for altering a cell property towards a property of one or more desired cell types comprising providing isolated RNA comprising a RNA sequence extractable from cells comprising said desired cell type(s) to a population of cells under conditions whereby the alteration of the cell property of said cells is achieved. In this method, the isolated RNA may be extractable from or extracted from one or more cell types that possess the property or properties of interest. The isolated RNA may comprise the sequence of RNA extractable from one or more cell types that possess the property or properties of interest. It is thus not always necessary to extract RNA from the desired cell types; the RNA sequence conferring the advantageous property or properties onto the cell type may be generated synthetically, for example, using a recombinant expression system. Larger quantities of the desired RNA may be produced by the in vitro expansion of isolated RNA. The population of cells may be exposed to the RNA in vitro, or in vivo. In vitro, the population of cells may for example be a cell culture, such as in a cell culture dish or roller bottle or cells growing on a support, membrane, implant, stent or matrix; or a tissue, such as an isolated tissue grown outside the body. In vivo, the population of cells may be an organism, such as a human patient, or a tissue isolated from an organism, such as an organ, a specific part of an organ, or a specific cell type or collection of cell types.

The present invention extends and builds upon the invention described in PCT/GB2004/00298.

The above method of the invention may be used to induce genotypic modification in cells, either in vivo or in vitro.

In one embodiment, the present invention provides a method of inducing genotypic modification in a cell, which comprises providing isolated RNA comprising RNA extractable from source tissue to the cell under conditions whereby the desired induction of genotypic modification is achieved. The RNA may be extracted or extractable from said source tissue. It is thus not always necessary to extract RNA from the desired cell types; the RNA sequence conferring the advantageous property or properties onto the cell type may be generated synthetically, for example, using a recombinant expression system. Larger quantities of the desired RNA may be produced by the in vitro expansion of isolated RNA.

The cells may be modified in vivo or in vitro. In one embodiment, therefore, this aspect of the present invention provides a method of inducing genotypic modification in cells in vitro, which comprises providing isolated RNA comprising RNA extractable from source tissue to a cell under conditions whereby the desired induction of genotypic modification is achieved. The RNA may be extracted or extractable from said source tissue. The target cells may for example be a cell culture, such as in a cell culture dish or roller bottle or cells growing on a support, membrane, implant, stent or matrix; or a tissue, such as an isolated tissue grown outside the body. Alternatively, the target sells may be in a tissue isolated from an organism, such as an organ, a specific part of an organ. The cells generated in vitro in this manner may be delivered into a recipient.

In another embodiment, this aspect of the present invention provides a method of inducing genotypic modification in a cell in vivo, which comprises providing isolated RNA comprising RNA extractable from source tissue to said cell under conditions whereby the desired induction of genotypic modification is achieved. The RNA may be extracted or extractable from said source tissue. For such in vivo treatment, the cell may reside and be exposed to the RNA in situ, in the body of the patient. Accordingly, the target cell may be in an organism, such as a human patient.

In these embodiments, cells and RNA may also be administered in simultaneous, separate or sequential application with other therapies effective in treating a particular disease.

Preferably, the target cells are totipotent, pluripotent or unipotent stem cells of a stem cell line or derived from a tissue of an animal or plant. More preferably, the target cells are totipotent, pluripotent or unipotent stem cells of a stem cell line or derived from a tissue of an animal, and in particular a mammal. More preferably still, the cells used are totipotent, pluripotent or unipotent stem cells of a human stem cell line or derived from a tissue of a human. In these embodiments, the present invention provides a method of inducing a further change in the stem cells' properties, namely differentiation into one or more desired cell types. For this additional alteration in cell property, the isolated RNA used in the method of the invention comprises RNA extractable from source tissue comprising the desired cell type(s). For in vitro treatment, this isolated RNA is provided to the cell culture of stem cells under conditions whereby the desired differentiation of said stem cells is achieved. For in vivo treatment, the isolated RNA is provided to stem cells in situ.

In another aspect, the invention provides for the use of the RNA capable of inducing genotypic modification of cells, particularly stem cells, in the treatment of, or in the manufacture of a medicament for improving or rectifying tissue or cellular damage or a genetic disease, including repair of diseased cells, alteration of the genetic constitution of cells, induction of specific cell types and cell fates, changing the immunological profiles of cells, and inducing particular desired immune functions or properties. The invention also provides for the use of the RNA additionally capable of inducing differentiation of stem cells in such uses. The isolated RNA may be provided to the cell population as a medicament in which the RNA forms the principal active ingredient of the medicament.

The isolated RNA may be used to induce genotypic modification of cells in vivo. Accordingly, in another aspect the invention provides a method of treatment comprising administration of the RNA capable of inducing genotypic modification of cells in a therapeutically effective amount to a patient in need thereof. The invention also provides for the use of RNA additionally capable of inducing differentiation of stem cells in such methods. Such methods may be used, for example, for the repair of diseased cells, induction of specific cell types and cell fates, alteration of the immunological profiles of cells, and induction of particular desired immune functions or properties.

The invention also provides cells obtained by the above methods. Such cells may be used in the manufacture of medicaments for treating a number of disorders. Thus, in a further aspect the invention provides for the use of the cells in the manufacture of a medicament for improving or rectifying tissue or cellular damage or degeneration or a genetic disease. The invention includes methods of treatment that comprise administration of these cells in a therapeutically effective amount to a patient in need thereof. Furthermore, the cells may be used for diagnostic and/or research purposes and/or in the manufacture of reagents used for diagnosis and/or research. Thus, in a further aspect, the invention provides for the use of the cells in diagnosis or research and in the manufacture of a reagent for diagnosis or research.

In some cases further desired genetic modifications may be introduced into the cells.

### BRIEF DESCRIPTION OF THE DRAWINGS

FIG. 1: The effects of brain RNA differentiated stem cells on age-related damage to the rat brain assessed by spatial learning and memory performance of recipient animals. Ex-breeder male rats aged between 468 to 506 days were given intravenously either untreated bone marrow stem cells or bone marrow stem cells treated with brain RNA extract. The results for control rats that received untreated stem cells (closed boxes) and those for experimental rats that received brain treated stem cells (open circles) are shown. The results show a remarkable increase in learning ability in the experimental rats.

FIG. 2: The effects of spine RNA differentiated stem cells on an animal model of motor neurone disease. SOD 1 mice were given intravenously either bone marrow stem cells treated with spine RNA extract, untreated bone marrow stem cells or physiological saline. The results for experimental mice that received spine RNA-treated stem cells (closed boxes), control mice that received untreated stem cells (open triangles) and control mice that received physiological saline (closed circles) are shown. The results show that pre-treatment of stem cells with spine derived RNA dramatically improved the efficacy of stem cell treatment in an established model of progressive neurodegenerative disease.

FIG. 3: The influence of donor tissue developmental stage on the effect of brain RNA differentiated stein cells on age related damage to the mouse brain assessed by spatial learning and memory performance of recipient animals. 254-299 day old C57/Bl mice were given intravenously either bone marrow stem cells treated with foetal (E15) brain RNA extract, bone marrow stem cells treated with adult (90 day) brain RNA extract or untreated bone marrow stem cells. The results for control mice that received untreated stem cells (closed boxes), experimental mice that received foetal brain treated stem cells (closed circles) and experimental mice that received adult brain treated stem cells (open triangles) are shown. The results show an increase in learning ability in the experimental mice, with the mice that received foetal brain treated stem cells demonstrating significantly faster learning.

FIG. 4: The effects of direct injection of bone marrow stem cell derived RNA on age related damage to the rat brain assessed by spatial learning and memory performance of recipient animals. Ex-breeder male rats aged between 433 to 570 days were given injections of either bone marrow stem cell RNA or bone marrow stem cell RNA treated with RNase into the right lateral ventricle. The results for control rats that received RNase treated stem cell RNA (closed boxes) and those for experimental rats that received stem cell RNA (open circles) are shown. The results show that control rats could not learn the task, while the stem cell RNA treated animals could learn the task with comparable performance to young rats.

FIG. 5: Views of cells treated according to Example 4 after 18 hours. A; Brightfield and B; Fluorescence views of cells treated with total RNA. C; Brightfield and D; Fluorescence views of cells treated with DNase. E; Brightfield and F; Fluorescence views of cells treated with RNase; G; Brightfield and H; Fluorescence views of control cells.

FIG. 6: Views of cells treated according to Example 4 after 72 hours. A; Brightfield and B; Fluorescence views of cells treated with total RNA. C; Brightfield and D; Fluorescence views of cells treated with DNase. E; Brightfield and F; Fluorescence views of cells treated with RNase; G; Brightfield and H; Fluorescence views of control cells.

FIG. 7: Views of cells treated according to Example 5 after 24 hours. A; Brightfield and B; Fluorescence views of cells treated with total RNA. C; Brightfield and D; Fluorescence views of control cells.

FIG. 8: Views of cell treated according to Example 5, 4 days after passage: A; Brightfield and B; Fluorescence views of cells treated with RNA; C; Brightfield and D; Fluorescence views of control cells.

FIG. 9: Views of cell treated according to Example 5, after passage #2: A; Brightfield and B; Fluorescence views of cells treated with RNA; C; Brightfield and D; Fluorescence views of control cells.

FIG. 10: Views of cell treated according to Example 19. A; Brightfield and B; Fluorescence views of cells treated with poly A+ RNA after 24 hours; C; Brightfield and D; Fluorescence views of control cells after 24 hours. E; Brightfield and F; Fluorescence views of cells treated with poly A+ RNA after 5 days; G; Brightfield and H; Fluorescence views of control cells after 5 days.

### DETAILED DESCRIPTION

The inventors have found that provision of RNA sequences from particular sources to cells can influence cell properties, and in particular cell genotype. Accordingly, the present invention is concerned with inducing genotypic modification in a cell, in vitro or in vivo. This is achieved by providing specific RNA sequences to the target cells.

In some embodiments, the “target cells” to which the specific RNA sequences are administered consist of a single cell type. However, in other embodiments, they may comprise more than one cell type, for example in the form of one or more tissues. This may particularly be the case when RNA of the invention is applied to cells in situ. The target cells may be any of the cell types described infra. In preferred embodiments, the target cells are stem cells, which may be any of the stem cell types described infra.

By “genotypic modification” is meant an inheritable alteration of one or more elements of the genome of the target cells. The alteration is from the sequence(s) found at said one or more elements in untreated target cells to the (different) sequence(s) found at corresponding elements in the genome of the source tissue. Such alteration may include substitution, insertion or deletion of one or more bases. The alteration may also include substitution, insertion or deletion of longer sequences within the element concerned, for example of 10, 100, 1000 or 10,000 bases, etc. The alteration may occur on the paternal and/or maternal chromosomes. The alteration may occur in any portion of the genome, e.g. within protein-coding regions (exons), non-protein coding regions, introns, promoter regions, ribozyme-coding regions etc.

Accordingly, the method of the present invention results in the cell undergoing a genetic transformation so as to acquire an altered, inheritable genotype. Such an altered genotype may reverse a genetic mutation that a cell has acquired through somatic mutation or which the cell has inherited. In this way, genetic disease may be treated or prevented. Such an altered genotype may provide a genetic change that provides for an additional, modified, removed or disabled function. This method of transformation is a form of gene therapy whereby a cell is genetically altered, so that the alteration is passed to any progeny. Accordingly, the methods of the present invention can be used in gene therapy, either of somatic or germ line cells, for the provision of cells that are genetically altered. Further examples will be clear to those of skill in the art.

The skilled person will be aware of many diseases that have a genetic basis. The present invention provides a method of treating these diseases by effecting a genotypic modification in cells that carry the disease-causing genomic characteristics. As noted above, this may be achieved by producing genotypically modified cells, particularly stem cells, with the methods of the present invention ex vivo (i.e. in vitro) and administering the genotypically modified cells to a subject. Alternatively, genotypically modified cells may be produced in vivo. Examples of diseases that may be treated with the present invention include those listed in the following table. The relevant disease-causing gene and common mutations for each disease (representing the genomic region that is modified to wild-type sequence by the methods of the present invention) are also provided. Further examples will be clear to those of skill in the art.

| | Disease- | Common genetic |
|---|---|---|
| Disease | causing gene | basis/bases |
| Muscular | Dystrophin gene | Deletions and point |
| Dystrophy | | mutations. No |
| | | common mutation |
| Cystic | Transmembrane | DeltaF508 mutation |
| Fibrosis | conductance | other mutation |
| | regulator | |
| | (CFTR) gene | |
| Haemophilia A | FVIII gene | Many mutations, |
| | | including intron 22 |
| | | inversion, intron 1 |
| | | inversion, point |
| | | mutation, small |
| | | insertion/deletion, |
| | | large deletion and |
| | | splice site mutation |
| Haemophilia B | Factor IX gene | No common mutation. |
| | | Most mutations in |
| | | Exon h |
| Sickle Cell | Hemoglobin | Several hundred HBB |
| Anaemia | beta (HBB) gene | gene variants known, |
| | | most common is HbS |
| | | variant |
| Cancer | Many, examples | Various - mutations, |
| (General) | include: | deletions, insertions, |
| | p53 | splice mutations etc |
| | K-ras | |
| | APC | |
| | DPC4 | |
| | p16 | |
| Cancer | Many, examples | Various - mutations, |
| (Specific): | include: | deletions, insertions, |
| Melanoma | INK4a/ARF, B-RAF | splice mutations etc |
| Breast cancer | BRCA1, BRCA2 | |
| Renal cell | VHL | |
| carcinoma | HER-2, C-MYC | |
| Ovarian cancer | | |

As used herein, “source tissue” means one or more tissues or one or more cell types in the RNA donor.

Preferably, the source tissue comprises one or more cell types in common with the target cells to be treated. More preferably, the source tissue comprises the most abundant cell type that may be present in the target cells. More preferably still, the source tissue comprises the most abundant two or three cell types that may be present in the cells. Even more preferably, the source tissue comprises all of the cell types that may be present in the target cells. Even more preferably, the source tissue consists of all of the cell types that may be present in the target cells. As noted above, the target cells may be entirely homogeneous in nature, in which case the source tissue may consist of those specific cells.

As described above, in certain embodiments, the target cells may be stem cells. In such embodiments, the cells may also undergo differentiation towards a more specialized form or function. For example, the cell may differentiate from a stem cell towards an adult cell with a specialised function (for example, a hepatocyte).

The term “function” is meant to include any biological activity that is observed in the differentiated cell type. Examples of functions include those that are specific to a particular tissue, for example, brain (for example, cortex, cerebellum, hippocampus, retina, substantia nigra, subventricular zone), spinal cord, liver, kidney, muscle, nerve tissue (peripheral, central, neuronal, glial), cardiac tissue (for example, atrial, ventricular, valve, cardiac innervation), immune cells, blood, pancreatic tissue, thymic tissue, spleen, skin, and gastrointestinal tract, lung, bone, cartilage, tendon, hair follicle, sense organ (for example, ear, eye), any gland either endocrine, exocrine, paracrine, such as thyroid, thymus, pituitary, adrenal, pancreatic, reproductive system (for example, testicular, prostate, seminal vesicle, ovarian, uterine, fallopian mammary), dental, vascular, digestive tract tissues (for example, stomach, gall bladder, intestines, colon). At a more detailed level, the function of particular cell types within a tissue type may be of interest, for example within brain tissue, neuronal cells or cortical neurones or glial cells have more specialised functions within the brain. At a more detailed level still, desired functions may be at a molecular level, where it is desired for specific molecules to be expressed on the surface of cells, such as specific T cell receptors in the case of T cells of the immune system. It is not possible for any list of desired function to be exhaustive and equivalent functions that may be desired in each circumstance will be apparent to the skilled reader.

In these embodiments, the direction of differentiation will be determined by the source of the RNA provided to the stem cells. Typically, the differentiation will be directed towards one or more cell types found in the source tissue.

Accordingly, the present invention allows the direction of differentiation to be dictated towards a particular speciality. For example, the stem cells may be directed towards liver function, or more specifically, hepatocyte function.

The invention provides methods and medicaments for the controlled manipulation of any stem cell to induce the cell to differentiate into a desired differentiated cell type. Such methods include the induction of stem cells to differentiate into one or more desired adult cell types.

A stem cell may, for example, be induced to differentiate in order to achieve a specific terminal differentiated state. Using the methods of the invention it is also possible to ensure that the differentiated cells are immunologically compatible with the intended recipient. The ability to choose what type of cell to induce the stem cell to differentiate into means that it is possible to produce a variety of different cell types from a single stem cell or stem cell line. The RNA molecules of the invention, or differentiated cell types obtained, may be employed in treating, or in the manufacture of medicaments for treating, various disorders. In particular they may be used for improving or rectifying tissue or cellular damage or a genetic disease. The ability to influence cell fate using RNA allows diseased cells to be repaired, allows the genetic constitution of cells to be altered, allows specific cell types and cell fates to be induced, allows immunological profiles to be changed at will, allows the induction of particular immune functions and so on.

The following embodiments of the present invention are specifically envisaged.

### a) Combined Genotypic Modification and Differentiation of Stem Cells.

The present invention provides a method of simultaneously inducing genotypic modification and differentiation in stem cells, which comprises providing isolated RNA comprising RNA extractable from source tissue to a cell culture of said stem cells under conditions whereby the desired induction of genotypic modification and differentiation is achieved. The RNA may be extractable or extracted from the source tissue. The present invention also provides cells obtained or obtainable by this method.

For example, as described in more detail below, a culture of stem cells may be obtained and treated in vitro with RNA of the invention to provide a culture of differentiated but still dividing cells. The culture will comprise differentiated cells of the same type(s) as are found in the source tissue. Moreover, at least a fraction of these cells will have undergone genotypic modification. If necessary, the proportion of genotypically modified cells may be enriched, as described below. Similarly, the total number of cells may also be increased by maintaining the cells under non-confluent conditions. Alternatively, differentiated, non-dividing cells may be obtained by allowing the cells to reach confluency. The resultant cells may be administered to a subject or otherwise used.

In one preferred example of this embodiment, the cells may be used to treat a patient having a disorder caused by an inherited defect of haematopoietic cells, for example, one caused by a mutation (e.g. a single base mutation) in a single gene. In this example, the source tissue may be extractable from the bone marrow of a healthy donor. Preferably, the RNA will be fractionated such that it comprises only RNA that induces the desired genotypic modification (as described below). A sample of bone marrow may be obtained from the patient and cultured ex vivo, using conventional techniques to provide a culture of bone marrow mesenchymal stem cells. These stem cells may then be treated ex vivo with the RNA extract, as described below. For example, the cells may be incubated with the RNA under normal cell culture conditions. A fraction of the resulting culture, which is capable of bone marrow engraftment, may then be selected. Within this population of cells, those that are differentiated and genotypically modified may be isolated (e.g. by limiting dilution clonal culture or other suitable technique). These cells are then available to repopulate the patient's bone marrow, preferably after a suitable ablation procedure.

In another preferred example, differentiated murine cells carrying a specific mutation, e.g. a single point mutation, may be obtained. For example, murine muscle cells that carry a mutation in the dystrophin gene may be obtained. In this example, the source tissue may be cells of a mouse carrying the mutation of interest (e.g. muscle cells of mdx mice). The stem cells may be any suitable stem cells described infra, but are preferably from a sample of bone marrow obtained from normal mice, which are then cultivated ex vivo using conventional techniques to provide a culture of bone marrow mesenchymal stem cells. These stem cells may then be treated ex vivo with the RNA extract, as described below. For example, the cells may be incubated with the RNA under normal cell culture conditions. The resultant culture will comprise differentiated muscle fibres. A proportion of these fibres will carry the mdx mutation.

In a further preferred example, differentiated murine cells expressing exogenous genes may be obtained. For example, murine muscle cells that carry a gene for GFP expression may be obtained. In this example, the source tissue may be cells of a mouse carrying the gene of interest (e.g. GFP-expressing neurones from a transgenic mouse). The stem cells may be any suitable stem cells described infra, preferably from a sample of bone marrow obtained from normal mice, which are then cultivated ex vivo using conventional techniques to provide a culture of bone marrow mesenchymal stem cells. These stem cells may then be treated ex vivo with the RNA extract, as described below. For example, the cells may be incubated with the RNA under normal cell culture conditions. The resultant culture will comprise differentiated neurones. A proportion of these cells will express the GFP gene.

### b) Use of the Combined Genotypic Modification and Differentiation of Stem Cells in Methods of Treatment.

This embodiment is a modification of the embodiment described in a) above, wherein the target stem cells are used after genotypic modification has been achieved, but before differentiation has taken place. This is possible because of a lag between uptake of the RNA and differentiation of the cells. During this lag, the cells have a latent ability to differentiate. Moreover, the present inventors have discovered that if such cells are administered to a subject, they are capable of migrating to and integrating into tissue comprising the one or more desired cell types into which the stem cells will ultimately differentiate. This effect is described in detail in PCT/GB2004/002981.

Such cells may be obtained, for example, by administering the cells shortly after RNA treatment, but before extensive differentiation has taken place. The precise timing involved will depend, inter alia, on stem cell type and the nature of the RNA used and can be identified by routine experimentation for any given procedure.

In one preferred example of this embodiment, the cells may be used to treat a patient having a disorder caused by the mutation of a single base within a gene. For example, the cells may be used to treat a human patient having muscular dystrophy, which may be caused by the mutation of a single base within the dystrophin gene. In this example, the isolated RNA may be extractable from the muscle of a human donor, living or cadaveric, that does not carry the mutation. A sample of bone marrow may be obtained from the patient and cultured ex vivo, using conventional techniques to provide a culture of bone marrow mesenchymal stem cells. These stem cells may then be treated ex vivo with the RNA extract, as described below. For example, the cells may be incubated with the RNA under normal cell culture conditions. A suitable time after this treatment, the cells may be administered to the patient, for example by intravenous injection. At least a proportion of the cells will migrate, integrate and differentiate into patient muscle. Moreover, at least a proportion of these will have the non-mutated form of the dystrophin gene.

### c) Genotypic Modification of Iso-Organic Cells.

In some embodiments of the invention, the target cells (which may or may not be stem cells) will be iso-organic with the cells of the source tissue. By “iso-organic” it is meant that the source tissue comprises the same cell type or types as are present in the target cells, or the same cell type or types that can give rise to the target cells.

In some cases, the target cells will be a culture of dividing cells. For example, as described in more detail below, a culture of cells may be obtained and treated in vitro with RNA from iso-organic source tissue to provide a culture of dividing cells, in which at least a fraction have undergone genotypic modification. If necessary, the proportion of genotypically-modified cells may be enriched, as described below. Similarly, the total number of cells may also be increased by maintaining the cells under non-confluent conditions. Alternatively non-dividing cells may be obtained by allowing the cells to reach confluency.

In other cases, the target cells will be a quantity of non-dividing cells. For example, these cells could be directly treated in vitro to obtain genotypically modified cells, a proportion of which may be enriched, as described below.

The resultant cells may be administered to a subject or otherwise used, as described below.

### d) Genotypic Modification of Non-Iso-Organic Cells.

This embodiment is a modification of the embodiment described in c) above, wherein the target cells (which may or may not be stem cells) are specifically chosen such that they are not iso-organic with the cells of the source tissue. In this embodiment, it is preferred that the RNA used to effect the genotypic change in the target cells has been fractionated to remove RNA that may be capable of inducing cell differentiation, as described below.

### e) Direct Application of RNA to a Subject.

RNA of the invention may be administered directly to a patient in order to effect genotypic modification of target cells in situ.

In one preferred example of this embodiment, the RNA may be used to treat a patient having a disorder caused by the mutation within a gene, for example of a single base within a gene. For example, the RNA may be used to treat a human patient having muscular dystrophy, wherein the isolated RNA may be extractable from the muscle of a human donor, living or cadaveric, that does not carry the mutation. Preferably, the RNA will be fractionated such that it comprises only RNA that induces the desired genotypic modification, as described below. However, in some embodiments, the RNA will be total RNA, in which case the patient may derive an additional regenerative effect (as described in co-pending UK Patent Application, Agent's Reference G039686PT). At least a proportion of the target cells in the patient will undergo genotypic modification to correct the mutation. For example, in the specific example given above, muscle cells in the patient will be modified such that they have the non-mutated form of the dystrophin gene.

f) Direct Application of RNA to a Subject for the Treatment of cancer.

This embodiment is a modification of the embodiment described in e) above, wherein the genotypic modification is to one or more genetic lesions associated with cancer. Such lesions may be associated with cancers in general or they may be specific to the type of cancer suffered by the subject. They may also be lesions that are specific to the individual cancer suffered by the subject.

In this embodiment, the source tissue is preferably derived from non-cancerous tissue of the patient to be treated (i.e. it is “autologous” source tissue). Preferably, the RNA will be fractionated such that it comprises only RNA that induces the desired genotypic modification, as described below. In some embodiments, allogeneic source tissue may be used, in which case it is preferred for the genotype of the donor at the relevant region to be identical to the normal tissues of the recipient, so as to avoid the possibility of an unwanted immunological response.

### g) Creation of Genotypically Modified Cells.

The present invention provides a generally applicable method of providing genotypically modified cells. The cells may be any of the cells described herein. In particular, they may be stem cells. Preferably, the RNA will be fractionated such that it comprises only RNA that induces the desired genotypic modification, as described below.

The present invention also provides cells obtainable by such methods.

### h) Creation of Genetically-Modified Organisms.

The present invention also provides genetically modified organisms derived from a genotypically modified cell of the invention.

In particular, the present invention provides a method for producing genetically modified mammals.

In this aspect, the source tissue is derived from a member of the same species as the desired genetically modified mammal, but which has the desired genotype. Preferably, the RNA is fractionated such that it comprises only RNA that induces the desired genotypic modification(s), as described below.

The target cells are cells that are capable of being implanted into the uterus of a female of the species in question and producing viable embryos. Typically, the cells will be fertilised eggs. However, unfertilised eggs may also be used, although these must be fertilised before implantation.

Alternatively, the target cells are early embryonic stem cells, which may subsequently be used in a conventional early embryo system for producing genetically modified animals. For example, a suitable method is described in Murphy and Carter (1993) Transgenic techniques, principles and protocols, Humana Press Inc., NJ.

The target cells are treated in vitro in accordance with the method of the present invention. For example, the cells may be incubated with the RNA under normal cell culture conditions.

More than one genotypic modification may be effected in the target cells, for example by using source tissue that comprises cells with multiple genomic differences. Accordingly, the present invention provides a method for producing double knock-out mammals, for example.

After treatment with the RNA, the cells are used to produce a developed mammal by conventional methods, which would depend on the nature of the cells, as discussed above.

### Enrichment of Genotypically Modified Cells

A culture of cells treated by the methods of the present invention, wherein at least a percentage of the cells are genotypically modified, may be enriched for this fraction using any suitable method.

For example, the genotypically modified fraction may be enriched using single cell cloning. In this method, a sample of the cell culture is diluted until a single cell is contained in each of a number of compartments. The individual cultures are then grown for a period of time. A sample of each culture is tested to see if the required modification has taken place. Cultures consisting of clones that have been modified may then be pooled to provide a mixed culture of modified cells.

In another example, where the genotypic modification changes a surface characteristic of the cells that can be labelled (e.g. with a fluorescent ligand, such as a fluorescent antibody), the culture of cells may be enriched by appropriate labelling followed by isolation of modified cells (e.g. with a fluorescence-activated-cell-sorter).

In another example, where the genotypic modification changes a surface characteristic of the cells which can be bound by a ligand (e.g. with a biotinylated antibody), the culture of cells may be enriched by binding of the ligand followed by isolation of bound cells with a suitable capturing receptor (e.g. streptavidin). For example, a quantity of magnetic beads coated with the capturing receptor may be added to a culture of cells with the ligand present. The modified cells, which will be bound to the beads, may then be isolated in a suitable column using a magnetic field before elution by washing away the ligand.

### RNA Molecules

In order to produce the desired changes in cell properties, the invention employs specific RNA. In general, the RNA employed is one that comprises RNA extractable from tissues or cells comprising the cell type or types that it is desired to induce the target cell to have a cell property of Accordingly, the RNA is extractable from or extracted from source tissue comprising the genotype that it is desired to induce in the target cells, as discussed above.

Moreover, in embodiments where the aim is to induce differentiation of a stem cell into a desired differentiated cell type, the RNA provided to the target cell is typically an isolated RNA comprising a RNA sequence extractable from tissue or cells comprising the desired differentiated cell type or types. The isolated RNA may comprise a RNA extractable from or extracted from tissue or cells comprising the desired differentiated cell type or types.

The degree to which the source of the RNA is homogenous will be dictated in part by the specificity of the target cells and, in those embodiments where differentiation is involved, the type of tissue that is desired. The RNA may be extracted from, or the RNA sequence may be derived from, a particular tissue type, for example, brain (for example, cortex, cerebellum, hippocampus, retina, substantia nigra, subventricular zone), spinal cord, liver, kidney, muscle, nerve tissue (peripheral, central, neuronal, glial), cardiac tissue (for example, atrial, ventricular, valve, cardiac innervation), immune cells, blood, pancreatic tissue, thymic tissue, spleen, skin, and gastrointestinal tract, lung, bone, cartilage, tendon, hair follicle, sense organ (for example, ear, eye), any gland either endocrine, exocrine, or paracrine, such as thyroid, thymus, pituitary, adrenal, pancreatic, reproductive system (for example, testicular, prostate, seminal vesicle, ovarian, uterine, fallopian, mammary), dental, vascular, digestive tract tissues (for example, stomach, gall bladder, intestines, colon). Such tissues are made up of a number of different cell types e.g. constituent cells of brain tissue include various sub-types of neurones and glial cells, vascular tissues, connective tissues and brain-resident stem cells. RNA may be from a specific type of tissue in a particular location, such as a left tibia or left frontal lobe. Accordingly, a more homogeneous population of cells might include neurones and so where the desired cell fate is itself specific (for example, in the treatment of age-related brain disease), the RNA may be extracted from neurones, or the RNA sequence may be derived from neurones. More specifically again, the RNA may be from a specific neurone type such as cortical neurones. More specifically again, the RNA may be from a specific type of cortical neurones, such as dopaminergic cortical neurones. In embodiments such as these, the RNA is from a purified cell source.

Source tissue may be from the same organism as the target cells. Alternatively, source tissue may be from one or more donors that are not the same organism as the target cells.

In specific embodiments, source tissue may be a whole organism (e.g. a whole embryo, fetus or post-natal cadaver), limb(s), organ(s), part(s) of an organ, an organ from which specific sub-component(s) have been removed, a collection of specific sub-components from organ(s) or specific cell-type(s). In other embodiments, source tissue may be an in vitro culture of one or more cell types, or one or more cell lines. Moreover, in any of these embodiments, the source tissue may have had specific cell type(s) (such as cells with one or more particular cell surface makers) completely or partially removed. Similarly, the source tissue may have had specific cell type(s) (such as cells with one or more particular cell surface makers) enriched, or be a selection of such cells.

In some embodiments, the RNA employed in the invention, derived from a particular tissue type or set of cells or cell lines or cell types, or a cell line or a single cell type, or the RNA sequence derived from such sources, may in addition use a source of such material which comes from a donor of a specific developmental stage. Accordingly the RNA may be derived from neurones from a particular developmental stage, where that developmental stage is the same as, or earlier than, or later than, the developmental stage of the intended recipient. Developmental stages include embryo, foetal, neonatal, juvenile, or adult, or any sub-stage of any of these stages.

In some embodiments the RNA employed in the invention, for the treatment of a tissue or organ in a recipient of a certain developmental stage, may be derived from a tissue or cell type or types that is related to that of the target cells, but where the exact type of source tissue is only present at a different developmental stage. For example, dental tissue in an adult might be treated with RNA derived form the emergent dental tissue in a neonate or young juvenile.

Other preferred sources of homogenous, purified RNA for use in accordance with the present invention include pure preparations of foetal, neonatal or juvenile cells and pure preparations of embryonic stem cells.

In some embodiments of the invention the RNA employed is derived from stem cells, and is administered into the whole organism, or organ, or tissue. In this case, the RNA provided is typically isolated RNA comprising RNA sequence extractable from a stem cell type or types or stem cell active tissue(s). The RNA may be extractable or extracted from a stem cell type or types or stem cell active tissue(s). Examples of stem cell-rich tissues include foetal tissue and embryo tissue, or tissues from later developmental stages undergoing a phase of growth repair or regeneration.

Typically, a cellular RNA extract will comprise a heterogeneous population of species of different RNA molecules. Types of RNA molecules in a heterogeneous population can include messenger RNA (mRNA), ribosomal RNA (rRNA), transfer RNA (tRNA), heterogeneous nuclear RNA (hnRNA), small nuclear RNA (snRNA), small cytoplasmic RNA (scRNA), small nucleolar RNA (snoRNA), transcription-related RNAs, splicing-related RNAs, signal recognition particle RNAs, linear RNA, circular RNA, inhibitory RNA (e.g. siRNA), single-stranded RNA, double-stranded RNA, etc. It may be desired to treat the cellular RNA extract so as to remove one or more types of RNA molecules that are either unnecessary or even detrimental to the methodology of the invention. In certain embodiments, a population of a small number of or only one RNA species may be prepared.

In a preferred embodiment the RNA will comprise, or consist essentiality of, a RNA extract from source tissue.

Thus, preferably a RNA rich extract is prepared from donor material. The donor material may, for example, be an organotypic source obtained post mortem. However, in other embodiments, the donor is the same organism as the target cells are from/in. Similarly, in those embodiments of the invention wherein stem cells are used, the donor material may be obtained from the same source as the stem cells to be treated.

In some embodiments, the source tissue may have been conditioned, modified, treated and/or cultured after extraction from the donor but before extraction of the RNA, such that the nature of the extracted RNA is altered in a desirable manner or the amount of active RNA present prior to extraction is increased. For example, the source tissue may have been treated to remove unwanted RNA sequences partially or completely. This may be achieved, for example, with interference RNA techniques. Alternatively, the source tissue may have been treated to increase the amount of active RNA present prior to extraction, for example by treatment with an appropriate medicament. The amount of active RNA present prior to extraction may also be increased by treatment with an RNase inhibitor (e.g. “RNA later” from Ambion) before or after removal from the donor, or after culturing, in order to reduce RNA degradation during the extraction process.

In some embodiments, the source tissue may be preserved at one or more points during the process of RNA extraction, for example by treatment with an RNase inhibitor and/or by storage at −20 to −80° C.

The RNA extract may be from an organ or tissue or cells isolated from an organ or a tissue. For example, the RNA extract may be from an organ, tissue or cells isolated from the group comprising, but not limited to, the brain, spine, heart, kidney, spleen, skin, the gastrointestinal tract or liver. In some embodiments, the source organ, tissue or cells may have been treated one or more times with the methods or medicaments of the present invention. The extract may be from a cell line of specific chosen phenotype, a primary cell culture, or a donor tissue of specific immunological profile.

In some embodiments, the source tissue may be cultured and the culture medium used as the source of, or as an additional source of, RNA of the invention. In such a culture, the cultured tissue may be treated in a particular fashion to modify the amount, activity and/or nature of the RNA released into the medium. For example, the inventors have found that subjecting a cultured tissue to mechanical damage can increase the amount of extractable active RNA. Moreover, in embodiments where a culture medium is used as a source of RNA of the invention, the RNA may be extracted from a fractionated part of that medium, For example, the RNA may be obtained by positive or negative selection of RNA associated with microvesicles, and in particular microvesicles with specific cell surface markers or composed of specific lipids. In another example, the RNA may be derived by positive or negative selection of RNA associated with particular proteins.

Similarly, in some embodiments, the source tissue may have been conditioned and/or modified before extraction from the donor, for example by artificial exercise of function (e.g. physical exercise of a muscle) or by treatment with medicament in order to alter the nature of the extracted RNA in a desirable manner.

Typically the RNA will comprise RNA sequence that is extractable from the same species as the target cell to be treated. Thus in cases where the target cell to which the RNA will be provided is an animal cell, the RNA will usually comprise a RNA sequence extractable from or a RNA extracted from an animal cell and in particular from the same species of animal as the target cell to be treated. Similarly, where the target cell is a plant cell, usually the RNA will comprise a RNA sequence extractable from or a RNA extracted from a plant cell and typically a plant cell of the same species as the target cell. However, in some embodiments, the source tissue may be derived from a xenogeneic source. Preferably, the target cells are human cells and the source tissue is from a human. However, in some embodiments, the source tissue may be from a non-human animal and in particular from a non-human mammal. In preferred examples of this embodiment, the source tissue may be from a pig. In other preferred examples of this embodiment, the source tissue is from a non-human primate such as a monkey. For example, the primate may be a chimpanzee, gorilla or orangutan.

The RNA may comprise a RNA sequence extractable from or a RNA extracted from any of the organisms or groups of organisms mentioned herein. The RNA may comprise a RNA sequence extractable from or a RNA extracted from any of the stem cell types or differentiated cell types mentioned herein.

In some embodiments, the RNA may comprise a RNA sequence extractable from or a RNA extracted from a different developmental stage than the recipient of the cells to be treated. For example, the developmental stage may be more immature than that of the recipient of the cells to be treated. Alternatively, the developmental stage may be a more active cell generative stage. For example, the treatment of spinal cord lesions may be effected by treatment with RNA obtained from donor embryo tissue, sourced at neuralation. The developmental stage may also be one that shows increased stem cell activity. For example, in some preferred embodiments of the invention, the RNA may comprise a RNA sequence extractable from or a RNA extracted from foetal, neonatal juvenile or embryonic developmental stages. For example, where the RNA is extractable from brain cells or tissue, the donor may be at a developmental stage when extensive neurogenesis is occurring, such as the foetal developmental stage. It has been demonstrated by the inventors that provision of RNA extractable from cells of an early developmental stage has advantageous effects, particularly in eliciting stem cell-mediated tissue repair.

The developmental stage may in alternative embodiments be less immature than that of the recipient of the cells to be treated or a less active cell generative stage. In some embodiments, the RNA may comprise a RNA sequence extractable from or a RNA extracted from a tissue that has been pre-treated (for example, chemically or physically) or pre-conditioned (for example, by exercise for muscle tissue or induction of a particular reproductive stage for reproductive tissue) in any way or ways which modify the activity of the extractable RNA. For example, the RNA may be extracted from tissue that has been stressed or damaged.

The alteration in genotype using the RNA in accordance with the invention as discussed above may result in the target cell adopting an immunological profile similar to or the same as that of the organism from which the RNA is extractable from. The expression “immunological profile”, is intended to include the immunological properties of the target cell in the intended recipient. Thus the invention may be used to change the immunological profile of a target cell in a desired manner. This may be used to ensure that the cells produced, or products produced from them, have a specific immunological profile. In particular, the RNA provided to the target cells may therefore be chosen so that the resultant cells, or products from them, have an immunological profile so that they are not immunogenic in the intended recipient or produce a minor immune response which is not significant and that preferably does not result in a detrimental phenotype. Thus the RNA provided may in a preferred case be a RNA sequence extractable from or a RNA extracted from, and particularly a RNA extracted from, cells or tissues of the intended recipient or an immunologically compatible subject. Such methodologies will in particular be useful in the provision of allografts or xenografts to patients, to minimise or prevent the risk of rejection.

The ability to change the immunological profile of a cell may mean that the stem cells or differentiated cells to which the RNA is provided do not themselves have necessarily to be immunologically compatible with the intended recipient. This means that cells such as stem cells may not necessarily have to be isolated from the intended recipient and, for example, already existing stem cells or stem cells from a more convenient source may be used. It may also mean that cells and in particular stem cells with a specific desired genotype may be employed and converted to a compatible immunological profile. For example, the intended recipient may have a genetic defect, whereas the stem cells or differentiated cells to which the RNA is provided may be from a different subject that does not have the same defect. Using the invention the donor cells may be rendered immunologically compatible to the intended recipient and also compensate for the genetic defect.

The alteration in genotype using RNA in accordance with the invention may therefore be used to change the immunological properties of cells, such that cells that are allogeneic or even xenogeneic with respect to the treated individual may be administered with a minimised risk of rejection of the cells. For example, pig cells treated with human RNA prior to injection may be introduced into human patients with a minimised risk of rejection, through alteration of the expression of cell surface molecules and their replacement with self molecules that would otherwise have been recognised as non-self by the treated individual. The isolated RNA may thus comprise a RNA sequence extractable from or a RNA extracted from a different species to that of the target cell to be treated.

The alteration in genotype using the RNA in accordance with the invention as discussed above may be used to boost the immune function of a diseased patient. For example, a RNA sequence for use in treatment may be isolated from a patient or species that is immune or relatively immune to the disease, either through natural resistance or through vaccination. The RNA may have the effect of conferring resistance to the treated patient, for example, through inducing a desired immune function or property already possessed by the cells of the individual from which the RNA was extracted. One example is in the incidence of pathogenic or viral disease. In such cases, it may be that RNA extracted from immune cells, such as T cells, of a resistant individual of the same or different species confers the required immune function to the treated individual. An example might be the case of HIV, which has little adverse effect on chimpanzees or certain groups of humans. RNA extracted from immune cells of chimpanzees or these groups of humans might be administered to a human or to immune cells isolated from a human and then reintroduced, in order to confer resistance on the human patient to AIDS.

The alteration in genotype using the RNA in accordance with the invention as discussed above may be used to reverse tumour growth. It is postulated herein that by exposing a tumour cell to a RNA sequence extractable from or a RNA extracted from a healthy cell, or a cell at an early developmental stage, the tumour cell may be induced to revert to a normal, healthy phenotype, or to become susceptible to elimination by the immune system or by genetic integrity maintenance systems for example, p53-mediated apoptosis.

### Preparation of RNA

Various techniques exist for the extraction of donor RNA. Such techniques may be used to obtain the RNA to be provided to the target cells. Alternatively, such techniques may be used to provide RNA to identify the sequences of the necessary RNA molecules in the RNA extract (e.g. by fractionation and screening). Thus the invention includes a method of screening for a RNA sequence capable of conferring a desired property from one cell type to another, comprising the steps of:
i. extracting RNA from cells comprising a desired cell type;
ii. separating the extracted RNA into different fractions;
iii. providing a fraction to one or more test cells and/or test recipients;
iv. analysing the test cells or recipients for an altered property possessed by the desired cell type from which the RNA was extracted;
   wherein a fraction that confers the altered property onto a test cell or recipient is identified as comprising a RNA sequence capable of conferring the desired property.

This screening method identifies RNA sequences that are capable of conferring a desired property from one cell type to another by fractionating the RNA extract and analysing RNA function using an appropriate assay. One example of an appropriate assay is an experiment of the type described in Example 3 below. The assay comprises providing isolated RNA comprising RNA extractable from cells comprising particular cell type(s) to a population of cells; and determining whether a cell property is altered towards a property of said desired cell type(s). In this way, RNA in an extract can be identified as unnecessary for the purposes of the invention and can be omitted (e.g. to simplify or standardise a RNA composition), ultimately leaving a RNA molecule, or set of RNA molecules, which are responsible for the desired activity.

Accordingly, the present invention also envisages the use of specific RNA sequences, specific RNA subtypes, or particular RNA structures that have been identified as capable of conferring a desired property from one cell type to another in the RNA extract. Such RNA molecules may be synthesised artificially. In some cases, the RNA may be an artificial or synthetic RNA or a RNA analogue based on the sequence of the extractable sequences. The analogue may be one chosen for its stability or ability to enter the target cell or other desirable properties.

Accordingly, the RNA employed in the invention is one that comprises RNA sequence extractable from tissues or cells comprising the genotype that it is desired to induce the target cells to have. Moreover, in the case where the aim is also to induce differentiation of a stem cell into a desired differentiated cell type, the RNA provided to the target cell may typically be an isolated RNA comprising RNA sequence extractable from tissue or cells comprising the desired differentiated cell type or types.

Suitable techniques include preparation by either cold or hot phenol extraction methodologies. Alternatively, the RNA may be sourced from specific tissues or cells by employing commercially available kits and in particular those that are based on the denaturing of protein and separation of RNA via centrifugation. For example, in one preferred protocol (cold phenol) extraction, primary donor tissue or cells is/are homogenised in a volume of physiological saline. An equal volume of 95% saturated phenol is added and initially centrifuged at 18,000 rpm in an ultra-centrifuge for 30 minutes. The aqueous phase is retained and brought to a concentration of 0.1 M MgCl₂ solution by the addition of 1M MgCl₂. Two volumes of ethanol are then added and this is allowed to precipitate for approximately 30 minutes. A final spin at 6,000 rpm for 15 minutes produces a RNA rich precipitate which can be retained and stored under ethanol. Alternatively, active RNA rich extracts may be prepared with any of the commercially available RNA extraction kits (such as, for example, RNAzol™). However, the precise methodology by which the RNA is extracted is generally not critical to the invention.

In some embodiments, the RNA used in the methods and medicaments of the invention will comprise “total RNA”, that is, a crude extract of RNA resulting from the extraction of essentially all types of RNA from the source tissue.

Alternatively, a specific fraction of a RNA extract may be employed. For example, the RNA population may be fractionated on the basis of size and a particular weight range of RNA species provided to the target cell. Fractionation may also be on the basis of weight, charge, or identifiable common chemical feature (for example, a structure, or the presence of a particular consensus or pattern of nucleotides) or any combination of size, weight or charge or common chemical feature.

In particular, the present inventors have ascertained that the active fraction of total RNA that effects genotypic modification in the target cells is the polyA positive fraction (i.e. the fraction of RNA that is polyadenylated). Accordingly, in preferred embodiments, the RNA used in the present invention is isolated polyA positive RNA in substantially pure form. By “substantially pure” is meant that the RNA consists essentially of isolated polyA positive RNA. However, as fractionation techniques based on the polyadenylation status of the RNA may not be 100% efficient, the RNA fraction may comprise a residual amount of polyA negative RNA. Isolated polyA positive RNA may be obtained from total RNA using any suitable fractionation techniques known in the art, for example as described above and in Aviv et al (1972) PNAS 69, 1408-1412, Sambrook and Russell (2001) Molecular Cloning. A Laboratory Manual (3^{rd} ed.) Cold Spring Harbor Laboratory Press, Cold Spring Harbor, N.Y. and reviewed by Homes and Korsnes (1990) Genet. Anal. Tech. Appl. 7: 145-150 and Jarret (1993) J. Chromatogr. 618: 315-339. Suitable methods include those that isolate polyA positive RNA by hybridisation of the polyA tail to a thymine oligomer coupled to a solid-phase matrix. In particular, isolated polyA positive RNA may be obtained using a commercially available separation kit, such as the Poly(A) Purist™ mRNA purification kit (Ambion cat#1916, see Ito et al (2003) Am J Path, 163, 2165-2172); the Poly(A) Purist™ MAG magnetic poly(A) RNA purification kit (Ambion cat #1922, see Hyun et al (2004) Mol Cell Biol 24, 4329-4340); or the μMACS™ mRNA isolation kit (Miltenyi Biotec cat# 130-075-102, see Fischer et al (2002) J Neuroscience, 22, 3700-3707).

In particular, polyA positive may be prepared according to the following protocol:

An extract of total RNA (comprising no more than 2,000 μg RNA) is resuspended in 0.75 cm³ nuclease free water and vortexed to resuspend the pellet. An equal volume of 2× binding solution (Poly(A) Purist™ mRNA purification kit, manufacturer's protocol) is added and mixed thoroughly. Each RNA sample is then added to a tube containing 100 mg oligo(dT) cellulose and mixed by inversion. The resultant mixture is then heated to 70° C. in a water bath for 5 minutes. After this time, the mixture is agitated gently for 60 minutes at room temperature. The oligo(dT) cellulose is pelleted by centrifuging the mixture at 3000 g for 3 minutes at room temperature. The resultant supernatant (which contains the polyA negative RNA) is then removed by aspiration and discarded.

In a washing step, 0.5 cm³ of Wash Solution 1 (Poly(A) Purist™ mRNA purification kit, manufacturer's protocol) is added to the oligo(dT) cellulose pellet and the mixture vortexed to resuspend the pellet. A spin column is placed in a 2 ml microfuge tube and the oligo(dT) cellulose suspension transferred to this column, which is then centrifuged at 3000 g for 3 minutes at room temperature. The filtrate is discarded from the microfuge tube and the spin column returned to the tube. This washing step is repeated a further time with Wash Solution 1 and a further three times with Wash Solution 2 (Poly(A) Purist™ mRNA purification kit, manufacturer's protocol).

The spin column is then placed in a fresh microfuge tube and 200 μl of warm THE RNA Storage Solution (Ambion cat#7001) (previously heated to 70° C. in a water bath) added to the oligo(dT) cellulose pellet. The mixture is vortexed briefly to mix the two and the tube immediately centrifuged at 5,000 g for 2 minutes at room temperature. This addition of warm THE RNA Storage Solution is repeated a further two times.

The spin column is discarded and 40 μl 5M ammonium acetate, 1 μl glycogen and 1.1 ml 100% ethanol added to the filtrate. This mixture (which contains the polyA positive RNA) is then stored at −70° C. for 30 minutes.

To recover the polyA positive RNA, the mixture is centrifuged at 12,000 g for 30 minutes at 4° C. and the supernatant removed by aspiration and discarded. The remaining pellet is then washed with 70% ethanol and vortexed. Finally, a polyA positive RNA pellet is obtained by centrifuging the resultant mixture at 12,000 g for 10 minutes at 4° C. This sample may be stored at −20° C. until required.

Accordingly, in some embodiments, the RNA will be fractionated total RNA. This may be obtained by one or more RNA fractionation techniques, as described in the following list. These techniques may be applied sequentially, each step involving the retention/removal of particular RNA fractions. Sometimes, it will be appropriate to pool fractions in a defined ratio before carrying out a further fractionation technique or in the production of the final RNA preparation for use in the methods and medicaments of the invention.

### Fractionation Techniques:

1) Fractionation according to the presence or absence of polyadenylation, for example as described above.
2) Fractionation according to mobility, for example by any suitable electrophoresis technique, for example as described in Pley et al. (1993) J. Biol. Chem. 268: 19656-19658 and Heus et al. (1990) Nucleic Acids Res. 18: 1103-1108.
3) Fractionation according to density, for example by any suitable centrifugation technique such as by sucrose gradient fractionation, described in Jain et al. (1997) Mol. Cell. Biology 17(2): 954-962.
4) Fractionation according to binding characteristics, for example by any suitable affinity purification technique such as described in Schnapp et al. (1998 Nucleic Acids Res. 26(13): 3311-3313.
5) Fractionation according to mass/size, for example by any suitable chromatography technique, as described in Lee & Marshall (1986) Prep. Biochem. 16(3): 247-58.
6) Fractionation according to the presence or absence of bound protein, for example by any suitable purification technique (e.g. immunological) that recognises the bound protein.
7) Fractionation according to inherent sequence information. The removal or enrichment of RNAs with particular sequence properties is specifically envisaged in the preparation of RNA for use in the present invention. For example, in some embodiments, it will be desirable to modify the content of the RNA extract by the selective removal or enrichment of RNAs of particular sequence. The selective removal or enrichment of RNAs of particular sequence may be achieved using any suitable complementary sequence RNA separation technique (for example, as described in Srisawat et al (2001) Nucleic Acids Res 29, E4).

In particular, the present invention provides a method for isolating from an RNA extract a fraction of RNA molecules that comprise a specific sequence. This method comprises the steps of:
i) contacting the RNA extract with one or more nucleic acid species capable of annealing to an RNA fraction in the extract;
ii) incubating the resultant mixture under conditions whereby said one or more nucleic acid species anneal with said fraction; and
iii) isolating the annealed fraction from the remainder of the extract, wherein said fraction is the fraction of RNA molecules that comprise the specific sequence.

The expression “nucleic acid” in step i) above typically means RNA, preferably synthetic RNA. However, in some embodiments, it may mean DNA, including cDNA, synthetic DNA or genomic DNA. The term “nucleic acid” also includes analogues of DNA and RNA, such as those containing modified backbones, and peptide nucleic acids (PNA).

Preferably, the one or more nucleic acid species of step i) are single-stranded, although in some embodiments double-stranded nucleic acids may be used.

In order to be capable of annealing to an RNA fraction in the extract, the nucleic acid species will typically comprise sequence that is complementary to a specific sequence found in the molecules of the RNA fraction to which they are to anneal. It will be appreciated that absolute complementarity, although preferred, may not be required for the species to anneal to the RNA fraction of interest. Accordingly, in some embodiments, the species may comprise sequence that is 99, 95, 90, 85, 80, 75, 70, 65, 60, 55 or 50% complementary to the specific sequence found in the RNA fraction of interest, provided that said level of complementarity allows the species to anneal with said molecules under the conditions used.

Generally, the sequence to which the species are complementary is only part of the sequence of the RNA molecules in the target fraction. Accordingly, the species need not be of the same length as the RNA molecules in the fraction. However, the species must be of an appropriate length to anneal selectively to RNA molecules comprising the complementary sequence. Designing suitable species for any given target RNA fraction would be routine to those skilled in the art. In general, the one or more nucleic acid species of step (i) are between 10 to 500 bases long. More preferably, they are between 10 to 250; 10 to 150; 10 to 100; 10 to 90; 10 to 80; 10 to 70; 10 to 60; 10 to 50; 15 to 40; 15 to 30 or 15 to 25 bases long. Even more preferably the one or more nucleic acid species are 17 to 25 bases long. More preferably, the one or more nucleic acid species are 20 bases long. Where more than one nucleic acid species is used, each species may be of a uniform length or they may have lengths that are independent of the lengths of the other species used. Preferably, they are of uniform length.

In some embodiments, the annealed fraction may comprise the RNA fraction for use in the present invention and will therefore be retained. However, in other embodiments, the non-annealed fraction will comprise the fraction for use in the present invention and the annealed fraction will therefore be discarded.

In a preferred embodiment, selective isolation of the annealed fraction is achieved as follows. The one or more nucleic acid species in step i) are coupled to one or more groups that are suitable for isolating the annealed fraction from the non-annealed fraction in the isolation of step iii). Examples of suitable groups include, but are not limited to, any tag that may be used for the purification of nucleic acids, such as biotin or a specific nucleic acid sequence. Nucleic acid fractions comprising such tags may be separated by contacting the fraction with (strep)avidin or a complementary nucleic acid sequence respectively. Those skilled in the art will be aware of other suitable tags and separation techniques for use in this embodiment.

This method for isolating from an RNA extract a fraction of RNA molecules that comprise a specific sequence may be used to select (positively or negatively) RNA sequences that induce specific genotypic modifications for use in the present invention. Accordingly, in some embodiments, the RNA used in the present invention may be enriched for (preferably to the extent that the RNA consists essentially of) RNA sequences that have the ability to induce one or more specific genotypic modifications in the target cells. For example, the present invention may be used to induce a genotypic modification wherein the genomic characteristics of a single gene in the target cells are altered to those of the source tissue. In another example, the present invention may be used to induce a genotypic modification wherein a short stretch of bases in the genome of the target cells is altered to that of the source tissue.

Similarly, in other embodiments, the RNA used in the present invention may lack RNA sequences that have the ability to induce one or more specific genotypic modifications in the target cells (for example, to the extent that the RNA used is substantially free of such sequences). This selective removal of RNAs of particular sequence may be desired when the unfractionated RNA comprises RNA sequences with the ability to induce one or more unwanted genotypic modifications in the target cells. For example, if the RNA is derived from a subject with a specific genetic mutation and it is desired for these mutations not to be induced in the target cells, RNA sequences capable of inducing the mutation may be removed. In another example, RNA sequences capable of inducing genotypic modifications to the genes defining one or more histocompatibility elements, e.g. MHCI, MHCII or other immune response-inducing proteins, may be removed.

The selective enrichment or removal of RNA molecules responsible for specific modifications may be achieved using the method described above, wherein the one or more nucleic acid species of step (i) comprise sequence that is capable of annealing with the RNA molecules responsible for the specific modifications. Accordingly, the one or more nucleic acid species of step (i) will typically comprise sequence that is complementary to sequence in the RNA molecules responsible for the specific modifications.

Where the sequence of the RNA molecules is unknown, it may be possible to design suitable species on the basis of the genomic modification that the molecules cause. Without wishing to be bound by theory, it is believed that an RNA molecule responsible for a specific modification will comprise one or more regions that are complementary to a sequence in one of the strands of DNA at the genomic region that is modified. The one or more regions that are complementary to a sequence in one of the strands of DNA at the genomic region that is modified may be complementary to the coding strand of the DNA. Alternatively, these one or more regions may be complementary to the non-coding strand of the DNA.

Accordingly, the one or more nucleic acid species of step (i) may comprise sequence that is complementary to a sequence of DNA at the genomic region in the source tissue corresponding to the genomic region modified in the target cells. The sequence that is complementary to a sequence of DNA at the genomic region in the source tissue corresponding to the genomic region modified in the target cells may be complementary to the coding strand of the DNA. Alternatively, this sequence may be complementary to the non-coding strand of the DNA.

Alternatively, in other embodiments, the species may comprise sequence that is complementary to a sequence of DNA at a genomic region some distance away from this region. For example, the complementarity may be to a strand at a region that is 10, 20, 30, 40, 50, 60, 70, 80, 90, 100, 200, 300, 400 or 500 bases away (upstream or downstream) from this region. In some cases, this may result in the species annealing with RNA molecules that do not comprise sequence complementary to the genomic region in the source tissue corresponding to the genomic region modified in the target cells. Instead, they may comprise sequence complementary to regions that flank said genomic region. Such molecules are not thought to be capable of effecting the specific genotypic modification when used alone. However, as discussed below, these molecules may be useful for improving the efficiency of genomic modification when used in combination with RNA molecules that comprise sequence complementary to the genomic region concerned.

As noted above, in some embodiments, the species used in step i) will be double stranded nucleic acid. Accordingly, in such embodiments, the species may comprise two strands, one complementary to one strand of DNA at the genomic region concerned and the other complementary to the other strand of DNA at the genomic region concerned. Such species will therefore comprise sequence that is complementary to the coding strand of the DNA and the non-coding strand of the DNA.

In some embodiments, more than one species may be used in step i). This may improve the efficiency of the isolation of specific RNA molecules. In such embodiments, each different species may comprise sequence that is complementary to adjacent sequences in the DNA at the genomic region concerned.

Where the specific genotypic modification of interest involves the alteration of only a few bases (e.g. less than 20 bases), a single nucleic acid species may be used in step (i), for example a single species of 17 to 25 bases, more preferably 20 bases.

In some embodiments, the specific genotypic modification of interest may involve alteration to a genomic region that is relatively long, for example between 100 and 500, 500 and 1000, 1000 and 5 000, 5 000 and 10 000, 10 000 and 100 000 or 100 000 and 1000 000 bases long. This may be the case when the modification involves the substitution or insertion of an exon or entire gene from the source tissue. In such embodiments, it is preferred to use more than one species in step i). For example, multiple species of 17 to 25 bases, more preferably 20 bases may be used. Typically, each different species comprises sequence that is complementary to adjacent sequence in the DNA at the genomic region concerned. These adjacent sequences may be spaced along the length of the genomic region concerned in order to reduce the number of species required. The optimal spacing for a given RNA target may be determined by routine experimentation. However, typical spacings would be between 100 and 1000 bases. The spacings may be uniform (i.e. all of the same length) or non-uniform.

In some embodiments, the species used in step i) may include one or more species comprising sequence that is complementary to a sequence of DNA at the genomic region in the source tissue corresponding to the genomic region modified in the target cells and one or more species comprising sequence that is complementary to a sequence of DNA that is at a genomic region some distance away from this region. This may result in the various species used in step i) annealing with multiple different RNA molecules in the RNA extract. As noted above, some of these RNA molecules may not comprise sequence that is complementary to the genomic region in the source tissue corresponding to the genomic region modified in the target cells. Despite this, the presence of these RNA molecules may improve the efficiency of genotypic modification. In particular, where the genotypic modification involves the insertion of a sequence that is not present in the target tissue, such RNA molecules may be essential for successful modification.

The sequence chosen for the species used in step i) may be used to probe the genome of the source tissue in silico to ensure that it is complementary to DNA at one genomic region. Preferably, species that are complementary to DNA at only one genomic region in the source tissue will be used in step i). This will limit the extent of non-specific binding to other RNA molecules that may be present in the RNA extract.

The number of different species required and the specific regions of DNA to which they are complementary may be determined for any given genotypic modification by routine experimentation.

Although the inventors have ascertained that the fraction of total RNA responsible for the effects of the present invention (i.e. the “active” fraction) is the polyA positive fraction, in some embodiments it may be desirable to further fractionate the RNA so that an even more pure sample of active RNA is obtained. This may be particularly the case in the methods of treatment and medicament of the present invention, where for regulatory reasons, inter alia, it may be necessary to administer a more defined preparation of RNA. Accordingly, in some embodiments, the RNA will have been further fractionated to concentrate the active fraction, for example by fractionating the RNA and carrying out a functional assay on the various fractions to identify the active fraction at each stage of the fractionation.

Accordingly, the RNA used in the present invention will preferably be one that is obtainable by the following procedure:
a) extracting RNA from source tissue;
b) separating the extracted RNA into different fractions;
c) providing each fraction to separate samples of one or more test cells;
d) analysing the test cells for an altered property possessed by the source tissue from which the RNA was extracted; and
e) retaining the fraction that results in a modified genotype in the test cells.

This procedure identifies RNA sequences that are capable of conferring a desired genotype from one cell type to another by fractionating the RNA extract and analysing RNA function using an appropriate assay. Any one or more suitable fractionation techniques from the list given above may be used in this procedure. Moreover, an example of an appropriate assay for use in this procedure is an experiment of the type described in Example 3 below. The assay comprises providing isolated RNA comprising RNA extractable from source tissue to a population of cells; and determining whether their genotype is altered towards that of the cells of the source tissue.

In some cases the RNA may comprise a mixture of sequences extractable from different cell types or tissues. For example, the RNA species may comprise a mixture of sequences extractable from two, three, four, five or more different cell types. In cases where it is desired to differentiate a stem cell, the RNA may, for example, be extractable from different cell types to produce a differentiated cell with characteristics of both cell types. In cases where the RNA is to be provided to a target cell that has a genetic defect, the RNA may be a mixture of sequences extractable from cells comprising and lacking the defect. For example, the RNA may comprise a blend of RNA extracts from cells from the subject with the defect and cells of the same type from another subject that lack the defect. In some cases specific sequences that are extractable from the desired cell type may not be present. For example, the transcript of a defective gene may be removed. The removal of specific sequences may, for example, be achieved, by selective degradation or by hybridisation. Ribozymes may be used to cleave specific sequences. RNase molecules may also be used with some degree of specificity.

In cases where the RNA is one extractable from a stem cell, preferred stem cells include any of those mentioned herein and in particular adult stem cells. The stem cell may, for example, be a haematopoietic, bone marrow stromal or neuronal stem cell. In cases where the RNA is one extractable from a differentiated cell, the differentiated cell may be any differentiated cell and may be in particular an adult differentiated cell. In a preferred embodiment the differentiated cell may be selected from a bone marrow cell, a neuronal cell, or a haematopoietic cell. The differentiated cell may be from any mammalian organ for example such as the kidney, liver, heart, pancreas, central nervous system, reproductive organ or other organ.

In some embodiments, isolated RNA extractable from cells and used in the methods of the invention is natural in derivation. By this is meant that the RNA contains no non-natural sequences and entirely consists of RNA from the species to which the cell belongs. In some embodiments, the RNA contains no viral, exogenous retroviral or pathogen sequences. In some embodiments, the RNA is a homogenous mixture and contains no siRNA, miRNA or other types of interfering RNA. In some embodiments, the RNA may not encode protein (e.g. the RNA does not have in-frame start and stop codons flanking a protein-coding region). In some embodiments, the RNA is not extractable from neoplastic cells. In some embodiments, the RNA contains no double-stranded RNA of a kind that directly activates an anti-viral immune response (e.g. by binding to a Toll receptor). In some embodiments, the RNA contains no antisense RNA (e.g. there is no RNA that is complimentary to the sense strand of a RNA transcript that is also present). RNA used according to the invention may be integrating or non-integrating. It may or may not be capable of replication. It may or may not have a 5′ cap. It may or may not act as a substrate for endogenous reverse transcriptase.

### Modified RNA and Analogs

The invention generally involves the use of RNA. This RNA comprises a sequence that can be extracted from cells comprising a desired characteristic. Transfer of the RNA to a target cell causes desired changes in the target cell, with the changes being defined by the RNA.

As shown herein, the RNA in which the changes are defined is active even when delivered within a phenol extract of RNA from a starting cell. This phenol extract contains a variety of different RNA molecules. If the activity is associated with specific RNA molecules and/or sequences within the extract then, to simplify preparation and quality control, it is preferred to deliver just the specific RNA rather than a complex mixture. The specific RNA can be prepared by purification from the RNA extract, or can instead be prepared synthetically or artificially (e.g. by chemical synthesis, at least in part, or by purification after transcription of the specific RNA from a template nucleic acid).

Accordingly, in addition to the use of RNA obtainable by extraction (optionally including additional fractionation, as discussed above), it is also specifically envisaged to use RNA prepared synthetically or artificially (e.g. by chemical synthesis, at least in part, or by purification after transcription of the specific RNA from a template nucleic acid) in the methods and medicaments of the present invention.

Thus the invention provides a process for preparing a RNA for use with the invention, comprising the step of synthesising the RNA by chemical means, at least in part. The invention also provides a process for preparing a RNA for use with the invention, comprising the steps of: contacting a template for said RNA with a RNA polymerase, whereby the polymerase can interact with the template to produce said RNA. The RNA polymerase could be a RNA-dependent RNA polymerase, but will typically be a DNA-dependent RNA polymerase (i.e. the template is preferably DNA, e.g. in the form of a plasmid).

The RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases. Bases such as pseudo-uridine, methyl-cytosine, and inosine may be present in such RNA molecules. It is also possible to include DNA nucleotides to form a DNA/RNA chimera. The use of modified backbones is a preferred feature of modified RNA molecules of the invention.

RNA analogs and mimics can also be used. Polymers that mimic natural RNA structures can be prepared and used with the invention etc. e.g. as described by Kirshenbaum et al. (1999). These modified molecules and analogs can be considered as “RNA” herein even if, from a strict chemical viewpoint, they are not simply ribonucleic acid.

### Amplified RNA

RNA obtainable by extraction (optionally including additional fractionation), as discussed above, or RNA prepared synthetically or artificially, as discussed above, may be amplified in vitro to increase the amount of active RNA present. Suitable techniques for this amplification, such as in vitro expansion of arbitrary RNA sequences, would be well known to those of skill in the art. For example, RNA may be amplified using the BD SMART mRNA amplification technique (Chenchik, A., et al. (1998) Generation and use of high-quality cDNA from small amounts of total RNA by SMART PCR in Gene Cloning and Analysis by RT-PCR (BioTechniques Books, MA), pp. 305-319).

### Provision of RNA to Target Cells

The RNA may be provided to the target cells in vitro or in vivo. The RNA may also be used in the manufacture of medicaments for the provision of the RNA to the target cells in situ. This is particularly the case where the RNA is provided to target cells in the animal mammalian body. In the case of plants the invention also provides methods for providing the RNA to the target cells both in vitro and in vivo. The RNA may be provided to the target cells by any suitable technique.

A number of methods for the provision of nucleic acid molecules to cells are known and these may be employed. For example, suitable techniques may include calcium phosphate transfection, DEAE-Dextran, electroporation, liposome encapsulation, liposome-mediated transfection, microsphere encapsulation, transduction using viral envelope particles and microinjection. The calcium phosphate precipitation method of Graham & van der Eb (1978) may be employed. General aspects of mammalian cell host system transformations have been described in U.S. Pat. No. 4,399,216 and may be employed. For various techniques for transforming mammalian cells, see Keown et al. (1990) and Mansour et al. (1988). In some cases the RNA or the enclosed RNA may be bound to chemical agents that enhance uptake by the target cells. For example the RNA of RNA-containing particles may be linked to an antibody specific to an appropriate receptor. Such a targeting chemical may increase uptake by all cell types, or may have an effect that is specific to a particular cell type or stem cell type. As an alternative, RNA can be administered without being bound to such reagents e.g. naked RNA. In some cases the RNA may simply be added to the culture medium of the cells for a suitable period of time. For example, the cells and RNA may be cultured together for from 1 minute to 10 days, preferably from 1 hour to 5 days, more preferably from 6 hours to 2 days. In a preferred embodiment the RNA may be cultured with the cells for 12 or 24 hours and in particular for 12 hours. In another example, the cells and RNA may be cultured together for prolonged periods from 10 days to 60 days. In a preferred embodiment, the RNA may be cultured with the cells for 10 to 14 days. In a further example, the RNA may be repeatedly administered to the cells hourly, daily or weekly. In another preferred embodiment, the RNA may be cultured with the cells for a short duration, for example from 1 minute to 6 hours and in particular for 1 hour. Similar time periods may be employed where the RNA is provided in the form of liposomes comprising the RNA sequences or by any of the other methods for providing RNA outlined above.

In some embodiments, the temperature of the cells in culture may be lowered or raised to facilitate uptake of the RNA. The cells are typically maintained at a constant pH. In some embodiments, the cells may be osmotically shocked to facilitate RNA uptake. The culture conditions may contain specified serum, or may be serum free. In some embodiments, the media may be conditioned by specific tissues or cell types. In other examples, the cells may be grown on a defined substrate (e.g. gelatin, polylysine, feeder cell layer etc.)

The amount of RNA provided to the target cells will be sufficient to bring about the necessary desired alteration in a cell property. For example the concentration of RNA may be from 10 ng to 5 mg/ml, preferably from 100 ng/ml to 2.5 mg/ml, more preferably from 1 μg/ml to 500 μg/ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. These concentrations may apply to in vitro or in vivo applications. In some cases, a total of 100 ng to 0.1 g, preferably from 1 μg to 50 mg, more preferably from 100 μg to 10 mg, still more preferably from 250 μg to 1 mg of RNA may be administered. Any suitable concentration and/or amount of RNA may be provided. A wide range of concentrations and/or amounts of RNA may be employed and the precise concentration and/or amount may be varied according to the method of delivery of the RNA to the target cells, the source of the RNA and whether the RNA is provided in vitro or in vivo. Once aware of the teaching of the present invention, it would be routine to the skilled reader to optimise the amount of RNA provided to the target cells in order to bring about the desired alteration.

The response of the cells to RNA of the invention may be enhanced by appropriate adjustment of the medium during treatment. For example, the medium may be adjusted to reduce RNA degradation. In one preferred embodiment, the medium is RNase-free. In another preferred embodiment, the medium contains an RNase inhibitor, preferably in saturating doses.

The RNA administered to a subject or used in the ex vivo treatment of cells may be extracted RNA as prepared according to any of the methods described above. However, in some embodiments, the RNA is first modified to increase its effectiveness or improve its in vitro and/or it vivo delivery. For example, suitable modifications include one or more of the following techniques.

### i) Microvesicle Packaging

The RNA may be contained in microvesicles (e.g. liposomes), in order to protect the RNA from RNase degradation and/or improve uptake.

Typically, for in vitro use, the composition and other characteristics of the microvesicles (e.g. size, wall thickness etc.) will be chosen to optimise the effective uptake of the RNA by the recipient cells (for example, by ensuring that the RNA is taken up in a cellular pathway that results in the desired effect), and/or to minimise degradation of the RNA during incubation. The composition and other characteristics of the microvesicles may also be chosen to facilitate the addition of ligands (described below) to enhance effective uptake by the recipient cells.

Typically, for in vivo use, the composition and other characteristics of the microvesicles (e.g. size, wall thickness etc.) will be chosen to optimise the effect of the RNA on the target cells (for example, by optimising effective uptake by the target cells and/or minimising uptake by non-target cells). The composition and other characteristics of the microvesicles may also be chosen to facilitate the addition of ligands (described below) to optimise the effect of the RNA on the target cells (for example, by optimising effective uptake by the target cells and/or minimising uptake by non-target cells).

### ii) Carrier Materials

The RNA may be conjugated or otherwise attached to carriers (e.g. polyethylene glycols of a particular molecular weight, sugars, lipids (e.g. cholesterol) or proteins) that protect the RNA from degradation, e.g. by RNase, and/or improve effective uptake.

Typically, for in vitro use, the nature of the carriers will be chosen to optimise the effective uptake of the RNA by the recipient cell and/or to minimise degradation of the RNA during incubation. Carriers may also be chosen to facilitate the addition of ligands (described below) to enhance effective uptake by the recipient stem cells.

Typically, for in vivo use, the nature of the carriers will be chosen to optimise the effect of the RNA on the target cells (for example, by optimising effective uptake by the target cells and/or minimising uptake by non-target cells). The nature of the carriers may also be chosen to facilitate the addition of ligands (described below) to optimise the effect of the RNA on the target cells (for example, by optimising effective uptake by the target cells and/or minimising uptake by non-target cells).

iii) Ligand Targeting Moieties

The RNA (optionally packaged in microvesicles or attached to a carrier as described above) may be complexed with a ligand that results in selective uptake by the target cells. For example, the RNA may be linked to an antibody or antibody fragment that targets the RNA to a desired population stem cells and results in effective uptake of the RNA by said cells.

### iv) Stored RNA

In some embodiments, the RNA of the invention may have been stored before use. In other embodiments the RNA may be used in treatment methods or in the manufacture of medicaments which will allow in vivo provision of the RNA to stem cells or to other cells. In such cases the RNA is typically formulated so that the medicament is in a suitable form for administration to the intended subject.

The medicament may be in a form where the RNA is in liposomes to facilitate delivery or alternatively encapsulated within viral envelope particles. The RNA may be present as naked RNA molecules or RNA molecules complexed with proteins and in particular proteins known to increase uptake of nucleic acids into cells.

The medicament may be administered in conjunction with other treatments given prior, simultaneously or subsequently which increase the time for which the medicament remains in an active state, in vitro or in vivo. For example the use of a known RNase inhibitor could be used for such treatment. Alternatively saturating dose of inactive or sacrificial RNA may be given to block the existing RNase activity.

The medicament may be administered in conjunction with other treatments given systemically or locally, prior, simultaneously or subsequently which increase the uptake or effect of the medicament in vitro or in vivo. For example molecules secreted in a local or systemic manner following tissue damage may enhance uptake of the medicament. Such molecules may originate from the damaged tissue per se, or from a stem cell source. In another example, known non-RNA inducers of tissue differentiation of specific tissues culture may be used in conjunction with the RNA of this method in vitro for example, the use of retinoic acid to aid differentiation of neuronal tissues. In another example known non-RNA supports of tissue culture may be used in conjunction with the medicament, for example, basic fibroblast growth factor in the culture of spinal neurons.

The medicament comprising the RNA may be delivered by any suitable route. For example, the medicament may be administered parenterally and may be delivered by an intravenous, rectal, oral, auricular, intraosseous, intra-arterial, intramuscular, subcutaneous, cutaneous, intradermal, intracranial, intratheccal, intraperitoneal, topical, intrapleural, intra-orbital, intra-cerebrospinal fluid, transdermal, intranasal (or other mucosal), pulmonary, inhalation, or other appropriate administration route. The medicament may be administered directly to the desired organ or tissue or may be administered systemically. In particular preferred routes of administration include via direct organ injection, vascular access, or via intra-muscular, intravenous, or subcutaneous routes. The RNA may be formulated in such a way as to facilitate delivery to the target cells.

The RNA may be provided on metallic particles. In some cases the medicament may be intended to be administered so that naked RNA is provided to the target cells. In cases where the RNA is provided present in liposomes or other particles, there may be targeting molecules present on the surface of the particles to allow targeting to the intended stem cells. For example, the particles may comprise ligands for receptors on the target stem cells or target differentiated cells. In one preferred embodiment, RNA is delivered to the cells via liposomes prepared after the methodology of Felgner et al (1987) Other suitable liposomes include immunoliposomes (e.g. U.S. Pat. No. 4,957,735).

RNA preparations may also be administered to an organism with cells, such as stem cells. Administration may be simultaneous, separate or sequential. Cells and RNA of the invention may also be administered in simultaneous, separate or sequential application with other therapies effective in treating a particular disease. In one embodiment, RNA extractable from one or more stem cell types or stem cell active tissue(s) may be administered in simultaneous, separate or sequential application with cells, such as stem cells. For example, in preferred embodiments, whole embryo RNA, foetal RNA, neonatal RNA or juvenile RNA is administered in simultaneous, separate or sequential application with stem cells, particularly bone marrow stem cells. It is shown here that stem cell mediated tissue repair and regeneration is improved by co-injecting embryo-derived RNA fractions with stem cells.

In embodiments where RNA is administered to a subject in vivo, the RNA is preferably administered by one or more of the following methods:

### i) Systemic RNA Application

The RNA of the invention may be provided by systemic application. For example, the RNA may be provided by intra-venous, intra-arterial, oral, intra-osseous or sub-cutaneous injection or infusion.

### ii) Localised RNA Application

The RNA of the invention may be provided to a restricted region in the body of the recipient. For example, application may be localised to an organ or limb. Generally, the restricted region will comprise the target cells to be treated.

In one embodiment, the region to which the RNA is provided may be defined by the circulation to that particular area of the body. For example, the RNA is provided by localised perfusion, or by infusion in one or more arteries supplying that particular area.

In another embodiment, the region may be defined by a distinct fluid region, such as the pleura, the peritoneum, the spinal cerebrospinal fluid or the ventricular cerebrospinal fluid. For example, the RNA is provided by localised injection or infusion into the fluid region of interest.

iii) Targeted RNA Application

The RNA of the invention may be provided in a manner that allows it to be preferentially taken up by a subset of cells or types of tissue, for example by the target cells. In these embodiments, the RNA may be modified such that it is preferentially taken up by the subset of cells or types of tissue. For example, the RNA may be packaged in liposomes that comprise specific ligands for a particular cell type and be injected systemically to provide targeted application to that cell type. Other examples of possible modifications for optimising effective uptake by the target cells are discussed above.

In this embodiment, the RNA may be provided by either systemic or localised application, as described above.

### iv) Multimodal RNA Application

RNA may also be applied by a combination of two or more of the above methods. In such embodiments, each administration may involve the same or different RNAs of the invention.

### v) Enhanced Application

RNA application may also be enhanced by administering one or more treatments to the subject that enhance the effective uptake the RNA by the target cells. These treatments may be systemic or localised, as discussed above. The treatments may also be given simultaneous, separate or sequential treatments in relation to the administration of the RNA. For example, in embodiments where RNA is administered by systemic intravenous injection, the recipient may be given an intravenous injection of an RNAse inhibitor either prior, simultaneously or after the RNA administration in order to reduce the rate of degradation of the RNA in the circulation. Alternatively, for example instead of RNAse in the above-noted example, the recipient may be given an effective amount of inactive RNA to sequester RNA-binding species and RNAase in the circulation.

### Stem Cells

In those embodiments of the present invention where the target cells are stem cells, any suitable stem cells may be used.

A stem cell is generally understood to be a cell capable of self-renewal that is also capable of differentiation into one or more specific differentiated cell type(s). Stem cells may be pluripotent, that is they may be capable of giving rise to a plurality of different differentiated cell types. In some cases the stem cells may be totipotent, that is they may be capable of giving rise to all of the different cell types of the organism that they are derived from. In other cases the stem cells may be unipotent (i.e. they may be “progenitor stem cells”), that is they may be capable of giving rise to one cell type of the organism from which they are derived. The invention is applicable to pluripotent stem cells, totipotent stem cells or unipotent stem cells, including the types of stem cells described below.

The stem cells may be plant or animal stem cells.

In a preferred case, the stem cells will be plant stem cells. Stem cells are known to occur in a number of locations in the seed and developing or adult plant. Stem cells differentiated or obtained in the present invention may be those from any of the tissues in which stem cells are present. Examples include stem cells from the apical or root meristems. In one preferred embodiment, the stem cells are from an agriculturally important plant. The plant may, for example, be maize, wheat, rice, potato, an edible fruit-bearing plant or other commercially farmed plant.

In another preferred case, the stem cells will be animal stem cells and preferably mammalian stem cells. In one preferred embodiment, the stem cells may be human stem cells. Alternatively, the stem cells may be from a non-human animal and in particular from a non-human animal. The stem cells may be those of a domestic animal or an agriculturally important animal. The animal may, for example, be a sheep, pig, cow, horse, bull, or poultry bird or other commercially-farmed animal. The animal may be a dog, cat, or bird and in particular from a domesticated animal. The animal may be a non-human primate such as a monkey. For example, the primate may be a chimpanzee, gorilla, or orangutan. The stem cells may be rodent stem cells. For example, the stem cells may be from a mouse, rat, or hamster.

Preferably, the invention uses mammalian stem cells from an adult, juvenile, baby, fetus or embryo. In some embodiments, for example when the use of stem cells derived from fetuses or embryos is prohibited by law, the stem cells may be from an adult, juvenile or baby.

Stem cells are known to occur in a number of locations in the mammalian body. Stem cells used in the present invention may be those from any of the organs and tissues in which stem cells are present. Examples include stem cells from the bone marrow, haematopoietic system, neuronal system, the brain, muscle stem cells or umbilical cord stem cells. The stem cells may in particular be bone marrow mesenchymal stem cells, foetal mesenchymal stem cells, blood-derived stem cells (e.g. CD34 positive circulatory stem cells), Tristem™ stem cells, spinal stem cells, neural stem cells, umbilical-cord derived stem cells, skin-derived stem cells, gut-derived stem cells, fat-derived stem cells and muscle-derived stem cells. Other examples include foetal-derived stem cells, placental-derived stem cells and tissue type-specific progenitor stem cells, such as retinal stem cells, liver stem cells, satellite cells, neuronal progenitors, glial progenitors, fibroblasts, olfactory ensheathing cells and reproductive system stem cells.

In a particularly preferred embodiment, the invention is used to differentiate adult human stem cells. The stem cells may in particular be bone marrow stromal stem cells, neuronal stem cells or haematopoietic stem cells, in a preferred case they may be bone marrow stromal stem cells or neuronal stem cells. In particular, when the methods of the invention are used to induce differentiation of a stem cell, the stem cell may be a bone marrow stromal cell.

In many cases, the RNA-treated stem cells may be intended to treat a subject, and in the manufacture of medicaments. In such cases the stem cells are preferably from the intended recipient (i.e. they are autologous stem cells). In other cases the stem cells may originate from a different subject. Accordingly, the stem cells may be allogeneic. However when the stem cells are allogeneic, they are preferably chosen to be immunologically compatible with the intended recipient. For example, the donor may be chosen to have an immunological profile which has a specific relationship to the immunological profile of the recipient. Accordingly, in some cases the stem cells may be from a relation of the intended recipient such as a sibling, half-sibling, cousin, parent or child, and in particular from a sibling. The stem cells may be from an unrelated subject who has been tissue-typed and found to have a immunological profile which will result in no immune response or only a low immune response from the intended recipient which is not detrimental to the subject. However, in other cases the stem cells may be from an unrelated subject as the invention may be used to render the stem cell immunologically compatible with the intended recipient. For example, the stem cell and the recipient may or may not have a histocompatible haplotype (e.g. HLA haplotypes).

In some cases the stem cells may be embryonic stem cells, foetal stem cells, neonatal stem cells, or juvenile stem cells. The embryonic, foetal, neonatal, or juvenile stem cells may be pluripotent stems cells and particularly totipotent stem cells. The cells may be from any stage or sub-stage of development, in particular they may be derived from the inner cell mass of a blastocyst (e.g. embryonic stem cells). The embryonic, foetal, neonatal or juvenile stem cells may be from, or derived from, any of the organisms mentioned herein. The embryonic, foetal, neonatal or juvenile stem cells may be human stem cells or non-human stem cells and in particular non-human animal stem cells (e.g. a non-human primate). The embryonic, foetal, neonatal or juvenile stem cells may be rodent stem cells and may in particular be mouse embryonic stem cells. In some cases, the embryonic, foetal, neonatal or juvenile stem cells may be recovered and then used in the manufacture of medicaments to treat the same subject, typically at some stage in their life. In one embodiment, where embryonic, foetal, neonatal or juvenile stem cells are employed, they will be from already established foetal, embryonic, neonatal or juvenile stem cell lines. This will particularly be the case for human cells. In some cases the stem cells may be obtained from, or derived from, extra-embryonic tissues. The stem cells may be obtained from the umbilical cord and in particular from umbilical cord blood.

In certain jurisdictions, for reasons of public policy, the stem cells may not be totipotent stem cells that have the capacity to form a human being. This is particularly the case where the stem cells are human foetal or embryonic stem cells.

The invention is also applicable to stem cell lines. Stem cell lines are generally stem cell populations that have been isolated from an organism and maintained in culture. Thus the invention may be applied to stem cell lines including adult, foetal, embryonic, neonatal or juvenile stem cell lines. The stem cell lines may be a clonal stem cell line i.e. they may have originated from a single stem cell. In one preferred embodiment the invention may be applied to existing stem cell lines, particularly to existing embryonic and foetal stem cell lines. In other cases the invention may be applied to a newly established stem cell line.

The stem cells may be an existing stem cell line. Examples of existing stem cell lines which may be used in the invention include the human embryonic stem cell line provided by Geron and the neural stem cell line provided by Reneuron. In a preferred case the stem cell line may be one which is a freely available stem cell, access to which is open, and in particular such an existing stem cell line.

In the case of human embryonic stem cell lines, in a preferred case a pre-existing stem cell line will be used. In a particularly preferred embodiment of the invention, where a human embryonic stem cell line is used, the cell line may be one where the derivation process (which begins with the destruction of the embryo) was initiated prior to 9:00 p.m. EDT on Aug. 9, 2001. Preferably human embryonic stem cell lines may be ones created from embryos donated for reproductive purposes which were no longer needed for the original purpose, because, for example, they were surplus to requirements. Preferably informed consent will have been obtained for the use of the embryos to create the cell line. In a preferred case, the human embryonic stem cell line employed will meet the requirements announced by President Bush on 9 Aug. 2001 as being necessary for obtaining US federal funding for embryonic stem cell research. These include the stem cell lines recognised as meeting the requirements from BresaGen Inc. of Australia; CyThera Inc.; the Karolinska Institute of Stockholm, Sweden; Monash University of Melbourne, Australia; National Centre for Biological Sciences of Bangalore, India; Reliance Life Sciences of Mumbai, India; Technion-Israel Institute of Technology of Haifa, Israel; the University of California at San Francisco; Goteborg University of Goteborg, Sweden; and the Wisconsin Alumni Research Foundation.

Reference herein to stem cell generally includes the embodiment mentioned also being applicable to stem cell lines unless, for example, it is evident that the target cells are freshly isolated stem cells or the stem cells are resident stem cells in vivo. The invention is applicable to freshly isolated stem cells and also to cell populations comprising stem cells. The invention may also be used to control the differentiation of stem cells in vivo.

An initial step in the methods of the invention may be the isolation of suitable stem cells. Methods for isolating particular types of stem cells are well known in the art and may be used to obtain stem cells for use in the invention. The methods may, for example, be used to recover stem cells from the intended recipients of the medicaments of the invention. Cell surface markers characteristic of stem cells may be used to isolate the stem cells, for example, by cell sorting. In particular embodiments, stem cells are obtained from tissue samples by a known method of culturing, for example the method of obtaining bone marrow mesenchymal stem cells from bone marrow or the method of obtaining cultures of CD34 positive stem cells from blood, bone marrow, spleen or liver. Stem cells may be obtained from any of the types of subjects mentioned herein and in particular from those suffering from any of the disorders mentioned herein.

The stem cells may be freshly isolated stem cells or they may be an ex-vivo culture of stem cells. They may also be obtained from one or more primary cell lines derived from any of the above examples. In some cases, the stem cells may be isolated from a subject, differentiated in vitro and then returned to the same subject. Such ex vivo methods are particularly preferred.

In some cases the target stem cells may be in situ, that is, they may be present in a subject. Such a method may, for example, be used for treating a degenerative brain disease or brain or spinal cord injury. It may also be used for the treatment of diseases such as liver disease, heart disease, skeletal or cardiac muscle disease and type I diabetes. Furthermore, it may be used to counteract age-related degenerative disease. Other examples will be clear to those of skill in the art.

In such embodiments, the stem cells may be any of the types of stem cells mentioned herein and may be in any of the organisms mentioned herein. The target stem cells may be present in any of the organs, tissues or cell populations of the body in which stem cells exist, including any of those mentioned herein. The target stem cells will typically be resident stem cells naturally occurring in the subject, but in some cases stem cells that have been transferred into the subject may be the target stem cells.

Various techniques for isolating, maintaining, expanding, characterising and manipulating stem cells in culture are known and may be employed. In some cases genetic modifications may be introduced into the genomes of the stem cells. Stem cells lend themselves to such manipulation as clonal lines can be established and readily screened using techniques such as PCR or Southern blotting. Techniques such as gene targeting or random integration may be used to introduce changes into the genome of the cells.

In some instances, the stem cells may originate from an individual with a genetic defect. Modifications may then be made to correct or ameliorate the defect. For example, a functional copy of a missing or defective gene may be introduced into the genome of the cell. Gene targeting may be used to introduce desired specific changes and in particular to modify a defective gene to render it normal. Site-specific recombinases may be used to remove selective markers involved in the gene targeting.

The stem cells used in the present invention may consist of a combination of two or more of any of the stem cell types described infra. In some embodiments, this combination may comprise a particular ratio of stem cell types.

In some cases the stem cells may be chosen because they have a specific genotype. For example the stem cells may be intended to produce differentiated cells to treat a subject with a genetic defect. The stem cells may lack the genetic defect. For example, the stem cells may be obtained from a relation of the subject who lacks the defect. For example, the cells may be derived from a sibling who does not have the disorder. Alternatively, the stem cells may be stem cells that have been treated ex vivo to correct a genetic defect. This is particularly preferred when the stem cells used in the methods and medicaments of the invention are derived from an intended recipient (i.e. they are autologous stem cells) who has said genetic defect.

The stem cells may be stored before use in the invention. For example, they may have been stored for between 1 hour and 1 year before use in the methods and medicaments of the invention, or alternatively between 6 hours and 6 months, 12 hours and 4 months, 18 hours and 3 months or 24 hours and 1 month. Suitable storage conditions include refrigeration at −80° C. in a suitable freezing solution or at −196° C. (liquid nitrogen) in a suitable freezing solution. An example of a suitable freezing solution would be 90% foetal calf serum, 10% DMSO. Preferred storage conditions are at −196° C. (liquid nitrogen) in a suitable freezing solution.

The stem cells may also have been treated ex vivo to preserve activity before use in the methods and medicaments of the invention. For example, the stem cells may have been treated by being cultured in appropriate media supplemented with LIF and β-mercaptoethanol to maintain them in an undifferentiated state (Bain (1995) Dev Biol 168, 342-357). In another example the cells may have been expanded in culture. In another example the stem cells may have been exposed to specific conditioned media. In another example the stem cells may have undergone a period of co-culture with a second cell type. In another example the stem cells may have been rejuvenated by exposure to appropriate RNA from a cell source of less chronological age or developmental stage.

### Pharmaceutical Compositions

The invention also provides pharmaceutical compositions comprising the various RNA molecules, RNA-treated cells and/or differentiated stem cells of the invention. The RNA molecules, RNA-treated cells and differentiated cells may be formulated with standard pharmaceutically acceptable carriers and/or excipients as is routine in the pharmaceutical art. Techniques for formulating cells and nucleic acids may be employed as appropriate. The cells or RNA may be provided in physiological saline or water for injections. The exact nature of a formulation will depend upon several factors including the particular substance to be administered and the desired route of administration. Suitable types of formulation are fully described in Remington's Pharmaceutical Sciences, 19^{th} Edition, Mack Publishing Company, Eastern Pennsylvania, USA, the disclosure of which is included herein of its entirety by way of reference. RNA-based pharmaceuticals are known in the art. For example, ‘Ampligen’ (Herispherx Pharma) is a medicament comprising double-stranded RNA molecules.

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sugars, and lipid aggregates (such as oil droplets or liposomes). Such carriers are well known to those of ordinary skill in the art. Compositions may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in Gennaro (2000).

Compositions of the invention will generally be in aqueous form (e.g. solutions or suspensions), but they may alternatively be in fried form (e.g. lyophilised). Liquid formulation allows the compositions to be administered direct from their packaged form, without the need for reconstitution in an aqueous medium, and are thus ideal for injection. Compositions may be presented in vials, or they may be presented in ready-filled syringes. The syringes may be supplied with or without needles. A syringe will include a single dose of the composition, whereas a vial may include a single dose or multiple doses.

Compositions of the invention may be packaged in unit dose form or in multiple dose form. For multiple dose forms, vials are preferred to pre-filled syringes. Effective dosage volumes can be routinely established, but a typical human dose for injection has a volume of 0.5 ml.

The pH of the composition for patient administration is preferably between 6 and 8, preferably about 7. Stable pH may be maintained by the inclusion of a buffer in the composition (e.g. a histidine or phosphate buffer). The composition will generally be sterile and/or pyrogen-free. Compositions may be isotonic with respect to humans. Compositions of the invention may include sodium salts (e.g. sodium chloride) to give tonicity.

Compositions of the invention may include an antimicrobial, particularly when packaged in multiple dose format. The various RNA preparations and compositions used to provide the RNA discussed herein to the target cell may also comprise agents to increase the stability of the RNA. For example, they may comprise RNase inhibitors or other agents that stabilise and/or protect the RNA from degradation. The RNA preparations may also have been treated to remove other kinds of molecules, for example protease or DNase treatment may have been used to remove protein and/or DNA. Thus the composition may be substantially free from DNA and/or protein.

Some pharmaceutical compositions of the invention include combinations of RNA extracted from source tissue according to any one of the embodiments described above, either alone or in combination with stem cells. The cells of the invention may be administered to a patient together with other active agents, such as one or more anti-inflammatory agent(s), anti-coagulant(s) and/or human serum albumin (preferably recombinant), typically in the same injection. The cells will generally be administered to a patient essentially in the form in which they exit culture. In some cases, however, the cells may be treated between production and administration. The cells may be preserved (e.g. cryopreserved) between production and administration. Cells may be present in a maintenance medium.

Specific combinations of particular interest include RNA extracted from brain tissue, neurone cells, cortical neurones and the like, with stem cells, for example bone marrow mesenchymal stem cells; spine RNA with stem cells, for example with bone marrow mesenchymal stem cells; foetal RNA with stem cells, for example with bone marrow mesenchymal stem cells; embryo-derived RNA, such as embryonic stem cell RNA with stem cells, for example with bone marrow mesenchymal stem cells. Examples of treatments would include: for Alzheimer's Disease treatment of bone marrow stem cells with foetal brain RNA; for treatment of Parkinson's Disease, bone marrow stem cells with RNA from a culture of dopaminergic neuronal cells obtained form a juvenile donor; for heart disease, bone marrow stem cells treated with RNA from a juvenile or adult cadaver; for diabetes CD34+ circulation stem cells treated with RNA from pancreatic islet cells form the cadaver of a normal adult. For multiple sclerosis, bone marrow stem cells treated with RNA derived from primary cultures of oligodendroglia. Such compositions are for simultaneous, separate or sequential administration to a patient suffering from a disease that is amenable to treatment according to the invention (although in each case treatment may also be effected by direct administration of only the RNA to the recipient). Examples of such diseases are presented above. Where stem cells and RNA are to be administered together, they may be packaged separately or in admixture, and they may then be administered separately or in admixture.

A therapeutically effective amount of the medicament, compositions, cells or RNA molecules will be administered to a subject. The dose may be determined according to various parameters, especially according to the substance used; the species, age, weight and condition, including immuno-status, of the patient to be treated; the route of administration; and the required regimen. A physician will be able to determine the required route of administration and dosage for any particular patient. The dose may be determined taking into account the age, weight and conditions of the subject to be treated, the type and severity of the degeneration and the frequency and route of administration.

The amount of RNA provided to the target cells will be sufficient to bring about the necessary desired alteration in a cell property. For example the concentration of RNA (e.g. in a composition of the invention) may be from 10 ng to 5 mg/ml, preferably from 100 ng/ml to 2.5 mg/ml, more preferably from 1 μg/ml to 500 μg/ml, even more preferably from 5 μg/ml to 100 μg/ml and still more preferably from 10 to 50 μg/ml. In a particularly preferred case the RNA concentration may be from 15 to 40 μg/ml, preferably from 20 to 35 μg/ml and in particular may be 25 μg/ml. These concentrations may apply to in vitro or in vivo applications. In some cases, a total of 100 ng to 0.1 g, preferably from 1 μg to 50 mg, more preferably from 100 μg to 10 mg, still more preferably from 250 kg to 1 mg of RNA may be administered. Any suitable concentration and/or amount of RNA may be provided. A wide range of concentrations and/or amounts of RNA may be employed and the precise concentration and/or amount may be varied according to the method of delivery of the RNA to the target cells or tissues, the source of the RNA and whether the RNA is provided in vitro or in vivo. It is routine to optimise the amount of RNA provided to the target cells in order to bring about the desired alteration.

The invention provides a pharmaceutical composition comprising a RNA of the invention (including RNA mimics, analogs, and modified RNAs), wherein the composition: (i) has a pH between 6 and 8; (ii) includes a buffer; (iii) is sterile; and (iv) is substantially pyrogen-free. The RNA in the composition is preferably homogenous. The RNA is preferably the active pharmacological agent within the composition. The composition is preferably located within a container that is labelled to indicate the composition's pharmaceutical purpose. The composition is preferably contained in an RNAse free container. The composition is preferably contained in a coloured bottle. The composition is preferably produced in an RNase free environment. The composition is preferably produced using reagents and chemicals that are essentially RNase free. The composition may comprise an RNase inhibitor.

### Medicaments and Methods for Treating Subjects

The cells and RNA provided by the invention may be used to treat a number of disorders, and in the manufacture of appropriate medicaments. The invention may employ a number of approaches to treat such disorders and to provide appropriate medicaments. In particular, administration of the medicaments of the invention to a subject to be treated may result in:
(a) administration of a RNA of the invention to a subject in order to induce genotypic modification of cells, such as stem cells, in situ; or administration of a RNA of the invention to a subject in order to induce genotypic modification and differentiation of stem cells, in situ;
(b) administration of genotypically modified cells obtained by the invention to a subject;
(c) administration of genotypically modified and differentiated stem cells obtained using the methods of the invention to a subject;
(d) administration of a RNA of the invention to the subject prior to, in conjunction with or after administration of cells (particularly stem cells), which cells may or may not have been altered according by the methods of the invention; and/or
(e) treatment of cells (particularly stem cells) with a RNA of the invention prior to, or after, administration of the cells to a subject.

In these embodiments, RNA may be provided to the chosen population of cells by providing the RNA locally, such as to an appropriate tissue or organ. For example, the administration of the RNA may be intravenous, rectal, oral, auricular, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, or by inhalation to a site of interest. The RNA may, for example, be provided by local injection. The RNA may be provided by injection into a blood vessel or other vessel that leads to the desired target site. The RNA may be administered by local injection to the desired tissue. The RNA may be administered by any of the routes mentioned herein such as intra-muscular injection or by ballistic delivery. In some cases the localised delivery may be achieved because the RNA is provided in a form that specifically targets the RNA to the target cells. For example, the RNA may be provided in liposomes or other particles that have targeting molecules for the specific desired stem cell type. In preferred embodiments the RNA may be administered via direct organ injection, vascular access, or via intramuscular, intra-peritoneal, or sub-cutaneous routes.

In one preferred embodiment administration of a RNA is achieved as follows:
a RNA extract is prepared from desired tissue type including any of those mentioned herein;
the RNA is injected either directly to affected organ or via systemic delivery as defined above; and
the RNA induces genotypic modification in resident cells

Similarly, cells may be delivered by providing the cells locally, such as to an appropriate tissue or organ. For example, the administration of the cells may be intravenous, intraosseous, intra-arterial, intramuscular, subcutaneous, cutaneous, intradermal, intracranial, intratheccal, intraperitoneal, topical, intrapleural, intra-orbital, intra-cerebrospinal fluid, intranodal, intralesional, transdermal, intranasal (or other mucosal), pulmonary, inhalation, to a site of interest. The cells may, for example, be provided by local injection. The cells may be provided by injection into a blood vessel or other vessel that leads to the desired target site. The cells may be administered by local injection to the desired tissue. The cells may be administered by any of the routes mentioned herein such as intramuscular injection. In preferred embodiments the cells may be administered via direct organ injection, vascular access, or via intramuscular, intra-peritoneal, or sub-cutaneous routes.

Preferably, the cells are preferably administered by one or more of the following methods:

### i) Systemic Cell Application

The cells may be provided by systemic application. For example, the cells may be provided by intra-venous, intra-arterial, oral, intra-osseous or sub-cutaneous injection or infusion.

### ii) Localised Cell Application

The cells may be provided to a restricted region in the body of the recipient. For example, application may be localised to an organ or limb. Generally, the restricted region will comprise the target cells to be treated.

In one embodiment, the region to which the cells are provided is defined by the circulation to that particular area of the body. For example, the cells are provided by localised perfusion, or by infusion in one or more arteries supplying that particular area.

In another embodiment, the region is defined by a distinct fluid region, such as the pleura, the peritoneum, the spinal cerebrospinal fluid or the ventricular cerebrospinal fluid. For example, the cells are provided by localised injection or infusion into the fluid region of interest.

### iv) Multimodal Stem Cell Application

Cells may also be applied by a combination of the above methods. In such embodiments, each administration may involve the same or different cells, which may or may not have been treated with RNA of the invention.

In the above methods of treating a subject the cells, genotypically modified cells, genotypically modified and differentiated cells, RNA, method of providing the RNA and other aspects may be as defined anywhere herein. In respect of the above agents, the RNA or differentiated cell or altered cell may be any defined herein.

### Provision of Cells

The invention provides cells obtained by the methods of the invention. The cells may be provided as frozen cells in a suitable receptacle. The cells may be provided in culture. Extracts of the cells are also provided such as whole cell extracts.

### In Vitro Methods for Inducing the Differentiation of Stem Cells

In those embodiments of the present invention involving the differentiation of stem cells, differentiation is achieved by providing the cells with a RNA sequence comprising a RNA extractable from the cell type that it is desired to differentiate the stem cell into. The RNA may be extractable or extracted from cells comprising said desired cell type(s). In particular the invention provides a method of inducing in vitro totipotent or pluripotent stem cells of a stem cell line or obtained from a tissue of an animal or plant to differentiate into one or more desired cell types, which comprises providing isolated RNA comprising a RNA sequence extractable from tissue or cells comprising said desired cell type(s) to a cell culture of said stem cells under conditions whereby the desired differentiation of said stem cells is achieved.

Any stem cell may be used in the methods, including any of those mentioned herein. In cases where the differentiated cells obtained are intended for use in the treatment of a subject, or in the manufacture of medicaments to treat a subject, the stem cells may originate from the intended recipient. In some cases the stem cells may originate from a recipient who has a genetic defect and preferably the genetic defect may have been corrected or ameliorated in the stem cells in such cases.

The RNA may be provided to the target stem cells using any of the methods discussed herein.

The stem cells may be induced into any desired cell type including any of those mentioned herein. In a preferred case the stem cell will be differentiated into a stable terminal differentiated cell type. A terminal differentiated cell type may generally be considered as one that does not naturally differentiate to give any other cell type and is typically at the end of a lineage. In some cases the stem cell may be differentiated into an intermediate cell between the stem cell and the terminal cell of the lineage. Such intermediates may have some degree of proliferative capacity.

The differentiated cell may be one of an organ or tissue such as the liver, spleen, heart, kidney, skin, gastrointestinal tract, eye, or reproductive organ. In a preferred embodiment the differentiated cell type may be one that is missing, present in reduced number or defective in a particular condition. The condition may be any of those mentioned herein and include injury, degenerative disease or a condition resulting from a genetic disorder. In a particularly preferred embodiment the differentiated cell may be an islet of Langerhans cell as the resulting cells can be used to treat diabetes. In another case the differentiated cell may be one of the central nervous system that can be used to treat a disorder or injury of the nervous system and particularly a disease of the brain or a spinal cord injury. In a preferred embodiment bone marrow stromal cells may be differentiated into neuronal cells.

In some cases the stem cell that is differentiated may be a pluripotent, but not totipotent, stem cell. In such cases the stem cell may, for example, be differentiated into a cell type that the stem cell is known to differentiate into in the organism it is isolated from.

In a preferred embodiment, bone marrow stromal stem cells may be differentiated into neuronal cells. In particular, they may be differentiated into neuronal cells expressing neuronal marker proteins (NeuN). Typically, the bone marrow stem cells may be differentiated into neuronal cells by providing an isolated RNA comprising RNA extractable from one or more types of brain cells or brain cell lines. In some cases the RNA may comprise a RNA extractable from brain tissue and in particular it may comprise a RNA extracted from a brain tissue. In a particularly preferred case the RNA may comprise RNA extractable from cortical neurones or a cortical neurone cell line. In some cases RNA extractable from neurones found in other locations than the brain may be employed or from cell lines derived from such neurones.

In another preferred embodiment, bone marrow stem cells may be induced to differentiate into muscle cells and in particular into skeletal muscle cells. Typically the RNA sequence provided will comprise a RNA extractable from or extracted from muscle cells or muscle cell lines and in particular from muscle stem cells.

In another preferred embodiment, pretreatment of bone marrow stem cells with spine derived RNA dramatically improved the efficacy of stem cell treatment in an established model of progressive neurodegenerative disease. Typically in this embodiment, the RNA sequence provided will comprise a RNA extractable from or extracted from spine cells or other cells in the peripheral nervous system. This methodology may also involve the administration of such RNA in vivo to influence the proliferation, migration and functional integration of stem cells in situ.

In another preferred embodiment, pre-treatment of stem cells with brain derived RNA has been shown to increase their proliferation, migration and functional integration into recipient nervous systems. Further, RNA sourced from a more immature developmental stage, at an active cell generative stage, appears to have a more profound effect on stem cell stimulation and their consequent ameliorative effect in both age and disease related damage. This methodology may also involve the administration of such RNA in vivo to influence the proliferation, migration and functional integration of stem cells in situ.

The invention provides cells obtained using the above methods. The cells may be provided in some cases as frozen aliquots in suitable receptacles. The invention also provides cell extracts of the cells.

In some cases the stem cells may be present in or on a structure such as a support, membrane, implant, stent or matrix when they are differentiated or alternatively the differentiated cells may be added to such a structure. The structure may then be used in the manufacture of a medicament for treating any of the conditions mentioned herein. Mixtures of different differentiated cell types may also be made, for example, to mimic populations occurring together in vivo.

In one preferred embodiment the in vitro method may comprise:
providing a stem cell population and culturing it in vitro according to established protocols;
providing RNA extracted from a desired target tissue type (for example neurones, glia, muscle or any of the differentiated cell types mentioned above) to the stem cells; and
maintaining the cells in culture.

In a further preferred embodiment the in vitro method may additionally comprise the step of
extracting RNA from a desired target tissue type (for example neurones, glia muscle or any of the differentiated cell types mentioned above).
   In these embodiments of the invention, the RNA may be preferably be provided to the stem cells either 1) as naked RNA extract 2) via liposome mediated transfer 3) by electroporation of recipient cells or other established methods.

Preferably the resulting differentiated cells may then be formulated into a medicament which can be administered to a subject by an appropriate route such as via the sub-cutaneous, sub dermal, intra-venous or intra peritoneal routes.

### General

The term “comprising” encompasses “including” as well as “consisting” e.g. a composition “comprising” X may consist exclusively of X or may include something additional e.g. X+Y.

The word “substantially” does not exclude “completely” e.g. a composition that is “substantially free” from Y may be completely free from Y. Where necessary, the word “substantially” may be omitted from the definition of the invention.

The term “about” in relation to a numerical value x means, for example, x±10%.

### MODES FOR CARRYING OUT THE INVENTION

The following Examples illustrate the invention.

### Example 1

### Production of Neural and Muscle Cells from Bone Marrow Stromal Stem Cells

### Marrow Harvest and Culture.

Bone marrow stromal (mesenchymal) stem cells were obtained from adult Sprague Dawley rats. The technique is based upon the protocol of Owen and Friedenstein (1988), and represents a typical established adult stem cell source suitable for expansion in vitro. Briefly, after schedule one killing (cervical dislocation), tibia and femora were excised within 5 minutes of death. All connective and muscular tissue was removed from the bones and all further procedures were conducted under sterile conditions.

Marrow was expelled from the bones by flushing the bones with media (α-MEMS—Gibco Invitrogen Co. UK) containing 10% foetal calf serum, and 1% penicillin/streptomycin. Flushing was achieved by inserting a 25-gauge needle attached to a 5 ml plastic barrel into the neck of the bone (cut at both distal and proximal end) and expelling 2 ml of media through the bone. The media and bone marrow sample were collected in sterile universal containers. Bone marrow cells were subsequently dissociated by gentle trituration through a 19-gauge needle approximately 10 times. One ml of aspirate was then placed in six well plates (SLS Ltd. UK). Two ml of fresh α-MEMS was then added to each well giving a plating density of approximately 12,000-15,000 cells per ml. Plates were then incubated at 37° C., in 5% CO₂ in air and left undisturbed for 24 to 48 hours (Harrison & Rae, 1997).

Following this time period, marrow derived stem cells were isolated from non-plastic adherent cells by aspirating the culture media from the plate. Plastic adherent marrow stromal stem cells remained, and were supported by the addition of 2 ml of fresh α-MEMS (10% foetal calf serum and 1% penicillin/streptomycin). New media was applied every 48 hours until the plate was confluent with colony forming units (CFU's) confirmed by microscope analysis (Owen & Friedenstein, 1988, supra). Under optimal conditions this required 5 to 7 days at 37° C. Resultant cells were confirmed as stromal stem cells morphologically and immunohistochemically.

### RNA Procedure

Brain homogenate was prepared and RNA separated using a RNA commercial separation kit or standard phenol based procedures. In the initial experiment, RNA was prepared by a cold phenol extraction method based on the method of Kirby (1956). Brains were freshly dissected from eight freshly killed rats. Eight grams of brain, excluding the cerebellum, was weighed and 5 ml of phosphate buffered saline (PBS) was added. The mixture was homogenised in a glass Teflon homogeniser for approximately 4 minutes. An equal volume of 95% saturated phenol was added. The resultant solution was left at room temperature for 15 minutes then centrifuged at 18,000 rpm in an ultra centrifuge for 30 minutes. The aqueous phase was retained and brought to a concentration of 0.1M MgCl₂ by the addition of 1M MgCl₂. Two volumes of ethanol were then added and precipitation was allowed to occur for approximately 30 minutes. A final spin at 6,000 rpm for 15 minutes produced a RNA rich precipitate, which was retained and stored under ethanol. Resultant RNA was air dried and dissolved in 6 ml of fresh media as defined above.

One ml of media containing the RNA was added to each well of confluent bone marrow stem cells for 24 hours. After 24 hours the RNA media was removed and replaced with fresh media. Cells were observed for phenotypic change every 12 hours.

Further, cells were subjected to immunohistochemical analysis to confirm that the RNA induced in the bone marrow stem cells was a neuronal phenotype. This was achieved by testing treated cells for the expression of a neuronal marker NeuN. The results obtained are indicated in the Table below.

| Cells | Morphology | NeuN |
|---|---|---|
| Untreated cells | Retained CFU morphology | − |
| Brain RNA treated cells | Developed Neuronal type morphology | + |

Examination of the cells showed the RNA induced change in cellular differentiation to a clear neuronal phenotype 24 hours after application of brain derived RNA. Untreated bone marrow stem cells retained the classic colony forming unit morphology. However, as early as 12 hours post-treatment the brain RNA treated stem cells showed typical neuronal and glial morphologies. Further, these cells expressed a commonly used immunochemical marker for neurones. Control cells did not. This change in phenotype survived passage (×3) and thus would appear a stable change in recipient stem cell differentiation. That donor tissue RNA was responsible for the change in stem cell differentiation was confirmed by subsequent experimentation in which the inductive effect of RNA was abolished by pre-treatment with RNaze, yet remained resistant to treatment of the donor brain RNA with trypsin, a potent protease.

The experiment was repeated using donor RNA, derived from skeletal muscle to confirm the specificity of the induced differentiation. It was clearly visible that the stem cells prepared as above and treated with muscle derived RNA (prepared using a commercially available kit, RNAzol), showed a stable differentiation change to muscle phenotype. This was confirmed by immuno staining with Phospholamban and Phalloidin. In the muscle study, the stem cells were exposed to muscle derived RNA (derived with a different RNA separation technique) via a different method of RNA delivery. RNA was delivered to the stem cells via liposomes prepared after the methodology of Felgner et al (1987). Thus it can be concluded from these studies that the induction in stem cells is specific to the donor tissue source, and that the RNA can be added to the stem cells via a variety of techniques commonly employed to deliver nucleic acids to cells.

### Example 2

### The Effects of Brain RNA Differentiated Stem Cells on Age Related Damage to the Rat Brain, Assessed by Spatial Learning and Memory Performance of Recipient Animals

Bone marrow mesenchymal stem cells were prepared in vitro as described above in Example 1. When the cells reached confluence, they were exposed to brain RNA (prepared as above) for 12 hours. Donor stem cells were derived from a pigmented rat strain (Lister Hooded). Donor RNA and recipient animals were provided from a different rat strain (Sprague Dawley).

Recipient Sprague Dawley rats were ex-breeder male rats aged between 468-506 days. It is well established that such animals of advanced age cannot learn to locate a hidden platform in a water maze (Stewart & Morris, 1993; Bagnall & Ray, 2000). Experimental animals received a 0.5 ml intra-venous injection of brain RNA treated stem cells, equating to the product of one six well plate of brain RNA treated cells. Control animals received an equivalent amount of untreated stem cells. Briefly, cells were collected from plates, either treated (experimental) or untreated (control) by mechanically removing them from the plastic plates using a rubber policeman and collected, by aspiration, in culture media. Cells were concentrated via a 5 minute spin at 1000 rpm and brought to a concentration outlined above. All injection procedures were conducted blind. For both groups, injections were mediated via the tail vein.

Fourteen days after injection, the aged rats were assessed blind on a commonly used spatial learning task, the Morris water maze. Each animal received 3 swims per day over a 3 day period with an inter trial interval of 10 minutes (Stewart & Morris, 1993). Latency to find the platform on each trial was recorded for each animal. Each trial consisted of a 60 second swim. If after that interval the animal had not located the platform, it was gently guided to the platform by the experimenter. Upon reaching the platform, the animal was allowed 10 seconds to orient to its location prior to removal to the home cage. Learning is evidenced by a decrease in time to locate the platform over repeated trials.

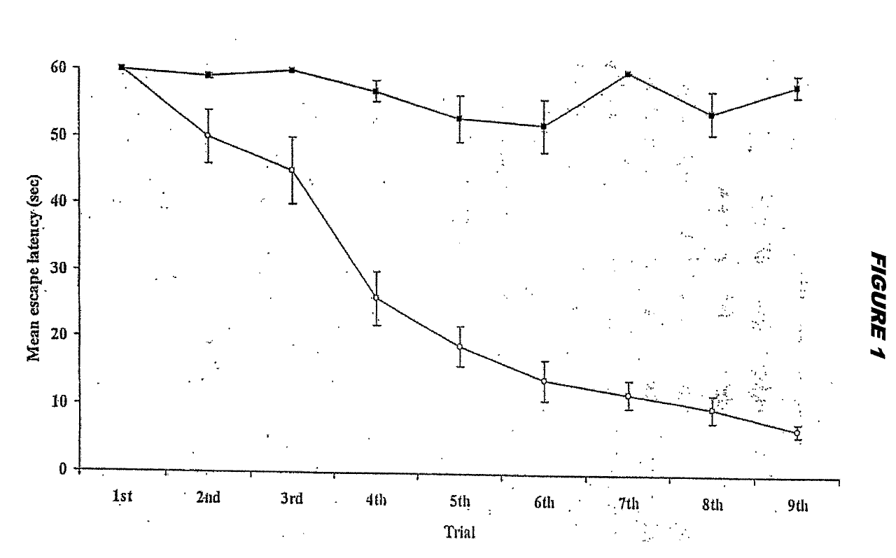

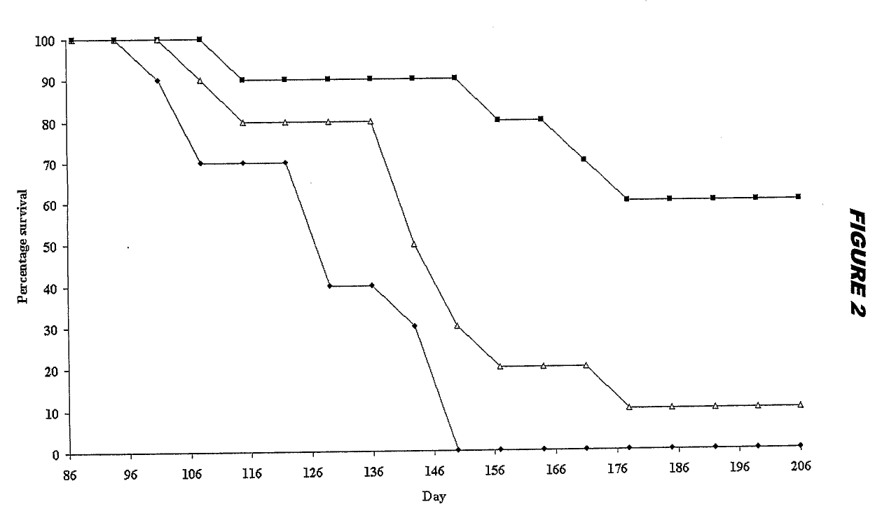

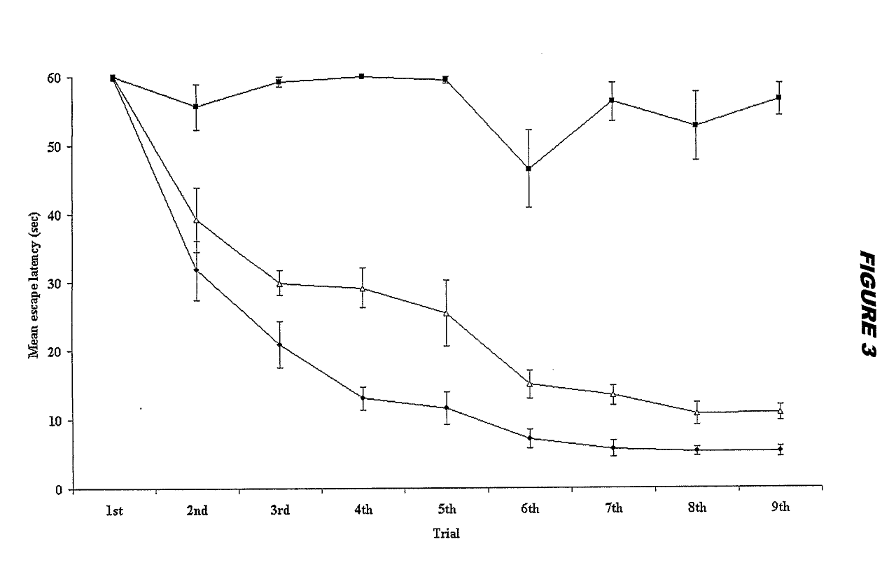

The results of the study are presented in FIG. 1. Control rats (n=9) receiving intra venous stem cells which had not been exposed to RNA, could not learn this task with no decrease in response latency over trials. However, the experimental animals receiving brain RNA treated stem cells showed a remarkable learning ability comparable to that of young rodents (p<0.0000000001). Two conclusions may be drawn from this study. First, RNA treated stem cells can significantly ameliorate age related deficits in spatial learning. Control untreated stem cells cannot. Second, it should be noted that donated stem cells were from a different strain of rat and recipient animals were not rendered immunodeficient. Thus, the results suggest that not only did the experimental group cells differentiate to appropriate neural tissue capable of functional improvement, they acquired an immunological status rendering them acceptable to the recipient. It should be noted that donor brain RNA was sourced from sibling animals to the recipients, yet donor cells were sourced from a different strain.

The results not only confirm that RNA differentiated stem cells can repair age related damage by restoring behavioural capabilities, but further that such treated cells acquire the immune characteristics of the donor RNA. This offers a strategy to change the immune profile of stem cell lines or stem cell banks to create differentiated cells with specific compatibility with the recipient.

### Example 3

### The Effects of RNA Extracted from GFP Expressing Mouse Brain on Wild Type Rat Bone Marrow Mesenchymal Stem Cells

Bone marrow mesenchymal stem cells were prepared in vitro as described above in Example 1.

Brain homogenate was prepared and RNA separated by commercial RNA separation kit or standard phenol based procedures. In the initial experiment, RNA was prepared by a cold phenol extraction method (Kirby). Brains were dissected from eight freshly killed GFP-expressing adult mice. Brains, excluding the cerebellum were weighed and 5 ml of phosphate buffered saline (PBS) added per gram of tissue. The mixture was homogenised in a glass teflon homogeniser for approximately 4 minutes. An equal volume of 95%-saturated phenol was added. The resultant solution was left at room temperature for 15 minutes then centrifuged at 18,000 rpm in an ultra centrifuge for 30 minutes. The aqueous phase was retained and brought to a concentration of 0.1M MgCl by the addition of 1M MgCl. Two volumes of ethanol were then added and this was allowed to precipitate for approximately 30 minutes. A final spin at 6,000 rpm for 15 minutes produced an RNA rich precipitate, which was retained and stored under ethanol. Resultant RNA was air dried and dissolved in 6 ml of fresh media as defined above.

One ml of media containing the RNA was added to each well of confluent bone marrow stem cells for 24 hours. After 24 hours the RNA media was removed and replaced with fresh media. Cells were observed for phenotypic and genotypic transformation every 12 hours. The results obtained are indicated in the Table below.

| | | GFP |
|---|---|---|
| Cells | Morphology | expression |
| Untreated cells | Retained CFU morphology | − |
| Brain RNA treated cells | Developed Neuronal type | + |
| | morphology | |

Within 24 hours of the addition of exogenous GFP RNA, the vast majority of adherent stem cells began expressing GFP, determined by fluorescent microscopy. Further, most stem cells began to show donor tissue (neural) morphology. In contrast, untreated bone marrow stem cells retained the classic colony forming unit morphology. This change in phenotype and genotype (to GFP expressing) was evident for at least 7 days post RNA exposure and survived passage (×3) and thus would appear a stable change in recipient stem cell phenotype and genotype.

In another example, this experiment was repeated using donor RNA derived from wild type B57/Bl mouse brains. This was applied to GFP-expressing bone marrow mesenchymal stem cells using the same methodology as above. In this example, recipient cells clearly lost GFP expression under the influence of wild type RNA. This change in genotypic expression was again stable over four days and through passage of the cells.

These data show exogenous RNA to be taken up by recipient cells in vitro and to be capable of stably changing recipient cell genotypic expression to that of the original donor tissue.

### Example 4

### RNASE and DNASE Treating the RNA Prior to Adding to the Cells

Rat bone marrow cells were passaged 24 hours prior to the experiment commencing, at a density of 8,000 cells/ml into 6 well plates.

The RNA used was from GFP rat brain. This was assayed on the NanoDrop for purity. The RNA was then split into two batches. One was treated with RNase, the other with DNase.

The RNase method was performed as follows:

To each RNA tube, 0.25 cm³ of RNase was added (at a concentration of 1 mg/cm³ in PBS), and incubated at 37° C. for 5 hours. After this time, 0.5 cm³ Bentonite was added at a concentration of 10 μg/cm³ in PBS. This was incubated for 1 hour at room temperature. The solution was then centrifuged for 10 minutes at 1500 rpm to precipitate the Bentonite, and the supernatant carefully removed as this is the fraction containing the RNase treated RNA.

The DNase method was performed using the Ambion TURBO DNA-free (cat#1907):

Each RNA sample was diluted in 1000 μl nuclease free water, and 100 μl of this was placed into a well of a 96 well plate. To this was added 10 μl DNase I buffer, followed by 2 μl rDNase I. This was incubated at 37° C. for 30 minutes, when a further 2 μl rDNase I was added and incubated at 37° C. for 30 minutes.

Two wells were then transferred to a 0.5 cm³ eppendorf, and 20 μl DNase inactivation reagent was added and incubated at room temperature for 2 minutes, vortexing a few times. Finally, this was centrifuged at 10,000 g for 90 seconds and the RNA containing supernatant was transferred to a fresh tube.

For the RNase treated group, the RNA concentration was originally 128 μg/ml

For the DNase treated group, the RNA concentration was 144 μg/ml

Untreated RNA bad a concentration of 128 μg/ml

The RNA was added in serum positive a-MEMS, and this was changed after 24 hours, and again after 48 hours.

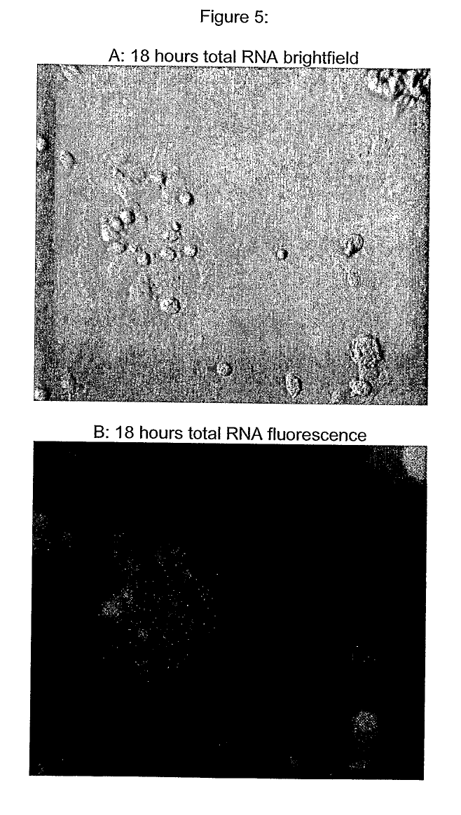

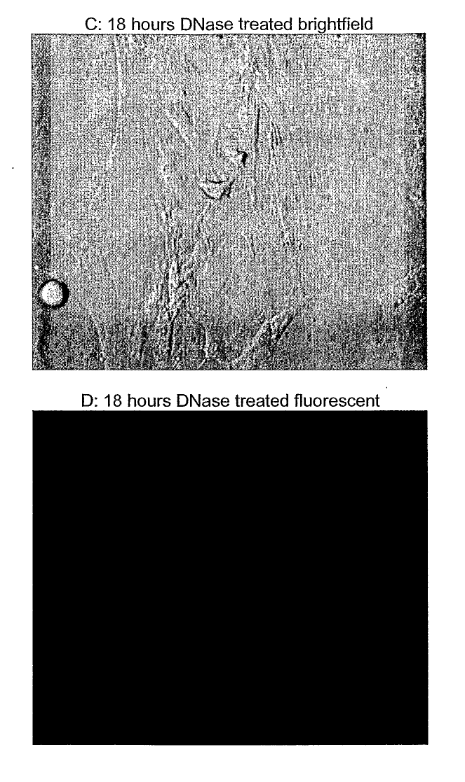

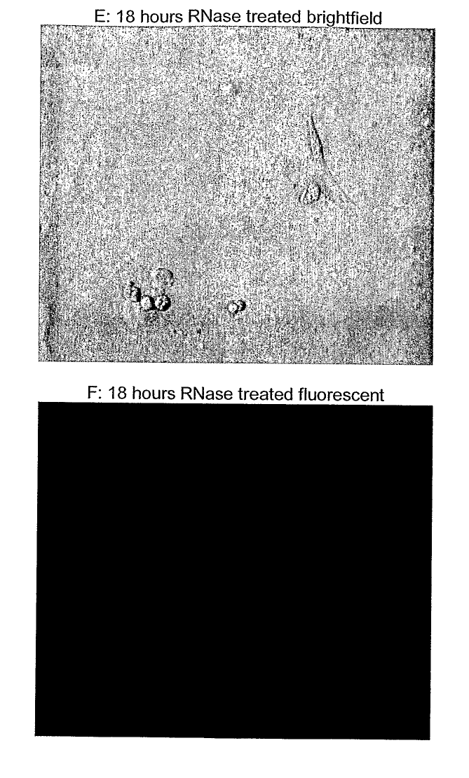

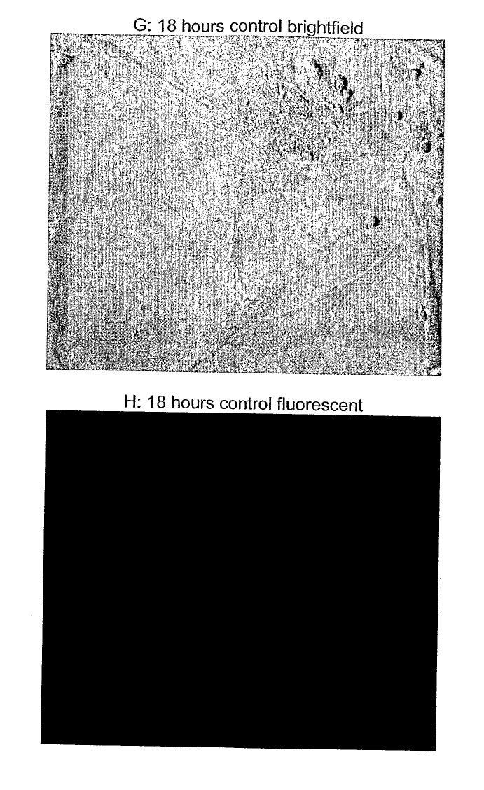

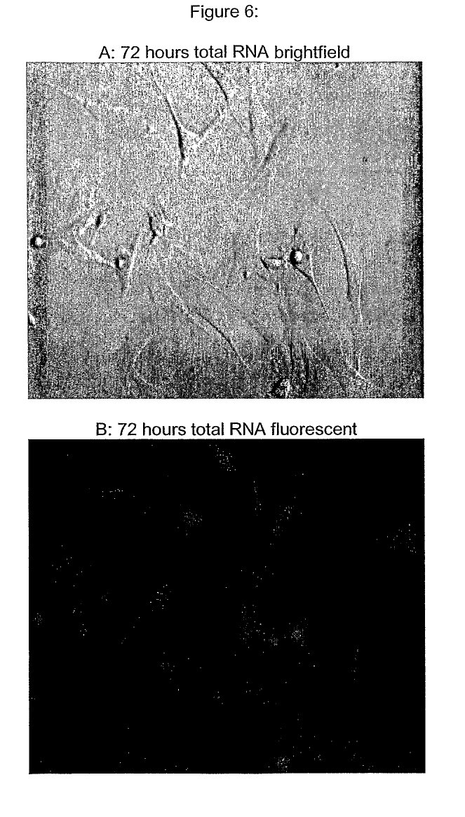

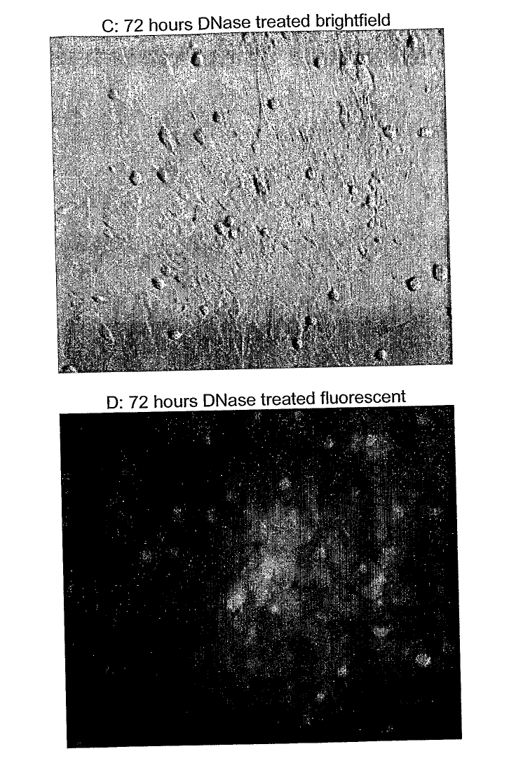

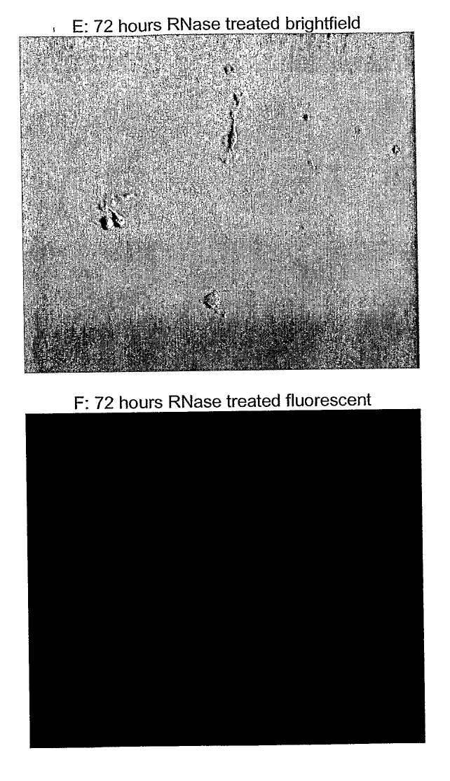

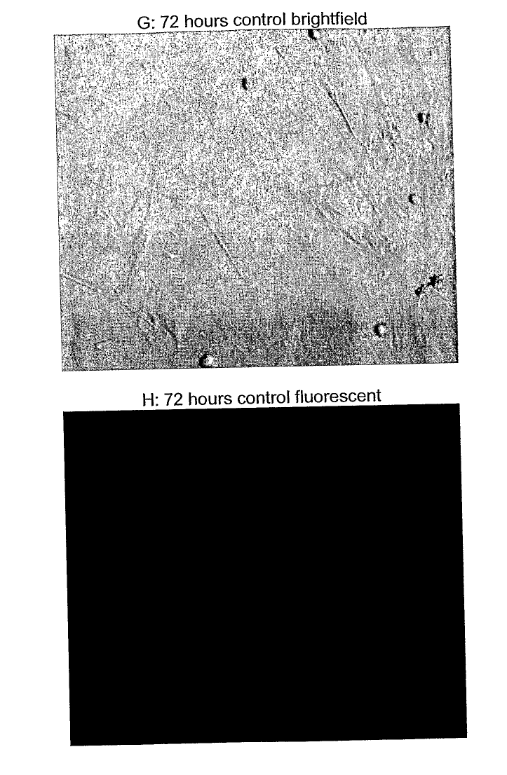

Photographs were taken in brightfield and fluorescence after 18 hours and then continuously thereafter. These are shown in FIGS. 5A to 5H and 6A to 6H.

The results showed that the DNase treated group were the only treated group to express GFP. This suggests that it is the RNA that is the active fraction. The Total RNA group also showed signs of fluorescence.

### Example 5

### The Effects of RNA Extracted from C166 Cells Expressing GFP on Wild Type Bone Arrow Mesenchymal Stem Cells and their Subsequent Drug Resistance to G418 Genetecin

Bone marrow mesenchymal stem cells were prepared in vitro as described above in Example 1.

The cells from eight 175 cm³ flasks of C166 GFP-expressing cells were removed and processed using an acid guanidium thiocyanate-phenol-chloroform RNA extraction procedure (Chomczynski et al (1987) Analytical Chemistry, 162, 156-159).

The tissue samples were reconstituted using solution D (0.36 ml 2-mercaptoethanol added to 50 ml Solution A). Solution A was made up with 250 g guanidinium thiocyanate, 293 ml distilled water, 17.6 ml sodium citrate (0.75M, pH 7) and 26.4 ml sarcosyl (10%).

The volume of solution D added was in the ratio of 1 ml:0.2 g tissue. This was then homogenised using a glass/Teflon homogeniser. After 10 passes, 10% of the volume of sodium acetate (2M, pH 4.0) was added and mixed by inversion. An equal volume to the solution D of phenol chloroform-iso-amyl alcohol (in a ratio of 25:24:1) was added and this mixture was shaken vigorously for 10 seconds, then cooled at −20° C. for 15 min. After this time, the solution was transferred to 1.5 ml tubes, with a maximum of 1.2 ml in each, these were then centrifuged at 10,000 g for 20 minutes at 4° C.

The top (aqueous) phase was then transferred to a fresh 1.5 ml tube, to a maximum of 0.5 ml, and 1 ml isopropanol was added. This was incubated at −20° C. for 15 minutes to precipitate RNA, and was then centrifuged at 14,000 rpm for 20 minutes at 4° C. The RNA pellet should be seen at the base of the tube. The supernatant was removed and 1.5 ml 100% ethanol was added, and the tubes vortexed, then chilled at −80° C. for 30 minutes. These were then centrifuged at 12,000 g for 20 minutes at 4° C. The supernatant was again removed, and the pellet was washed with 1 ml 70% ethanol, vortexed and centrifuged at 12,000 g for 10 minutes at 4° C. This can then be stored at −20° C. until required.

At this stage, the RNA purity and concentration was measured using a Genequant spectrophotometer. The average purity (A260/A280)=1.96, and the average concentration 72.72 μg/ml. This corresponded to a yield of RNA of 8.7 μg/106 cells.

The resultant RNA was air dried and dissolved in 6 ml of fresh media as defined above. One ml of media containing the RNA was added to each well of confluent bone marrow stem cells for 24 hours to give a final concentration of RNA of 96 μg/ml. After 24 hours the RNA media was removed and replaced with fresh media. Cells were observed for phenotypic and genotypic transformation every 12 hours.

The cells were passaged once a week for 4 weeks and after each passage the cells were monitored for their morphology. Again, after each passage, the bone marrow cells receiving the C166 GFP RNA expressed GFP as well as having the visible shape of C166 GFP cells. On the fourth week, the morphology of the bone marrow cells which received the C166 GFP RNA still retained the morphology of C166 GFP cells. This was not only due to the shape of the cells and the speed in which they colonised the wells, but they also fluoresced.

On passage 27, a sample of rat bone marrow cells with C166 GFP and C166 wild type RNA was analysed using PCR together with a C166 GFP cell type positive control.

The rat bone marrow cells with C166 GFP and C166 wild type RNA showed that GFP DNA was present in the cells. This was mirrored with the C166 GFP cells.

In equivalent experiments, cells were monitored daily and photographed in both brightfield and fluorescence.

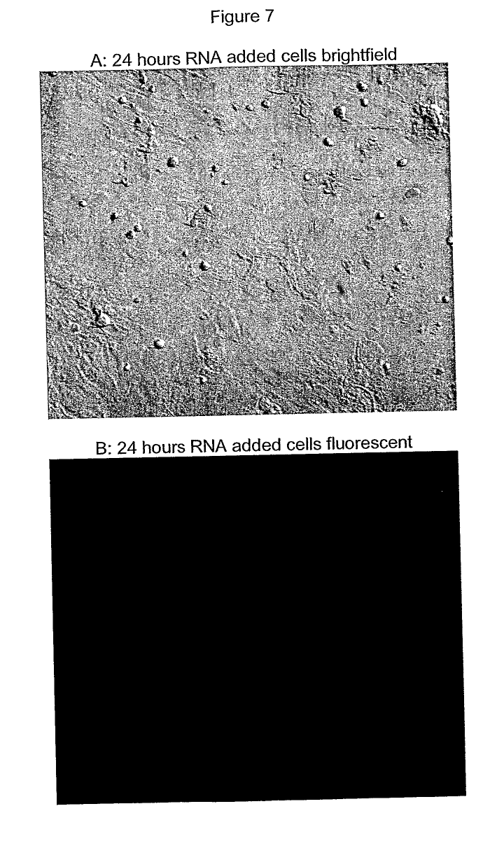

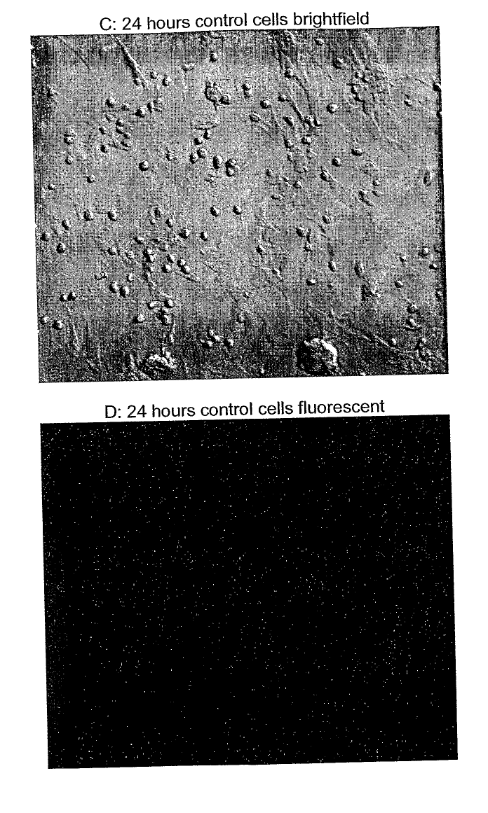

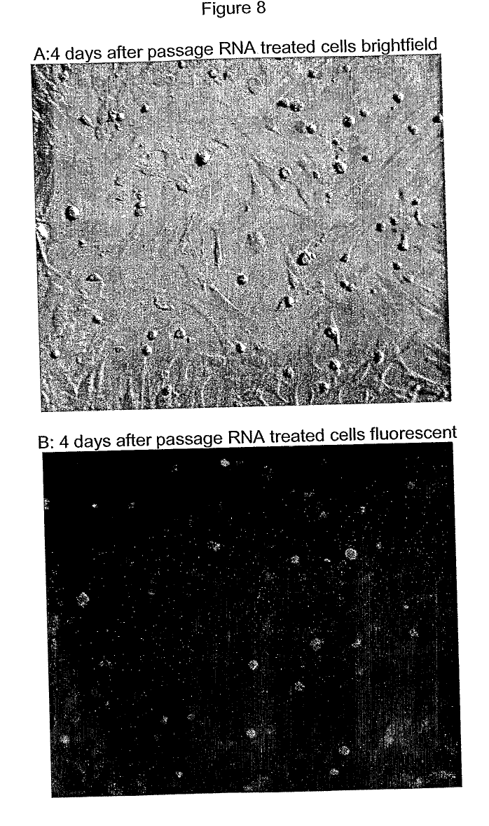

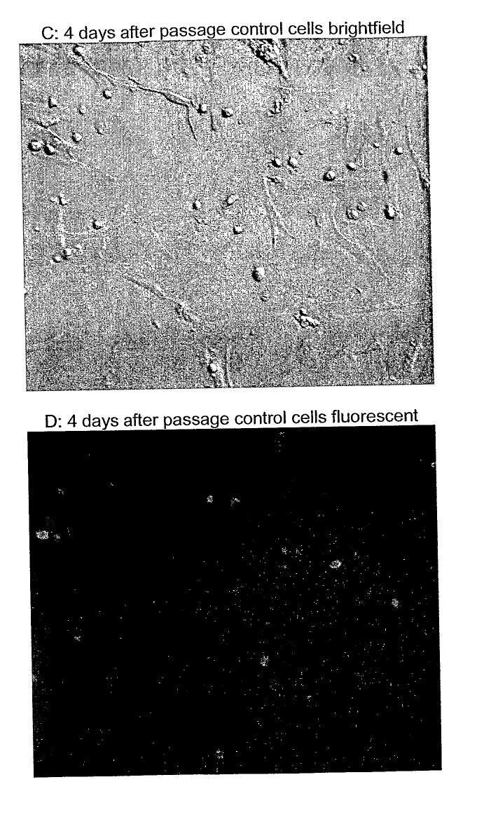

The cells showed no signs of differentiation or GFP expression after 2 days, when they were passaged into six well plates (see FIGS. 7A to 7H). Again, the results did not yield any positive results. Four days after passage, the cells receiving the C166 GFP/C166 wild type mixed RNA showed fluorescence, compared to the controls, where only auto-fluorescence was observed (see FIGS. 8A to 8H).

The original flasks were kept after the cells were passaged and they too showed signs of fluorescence.

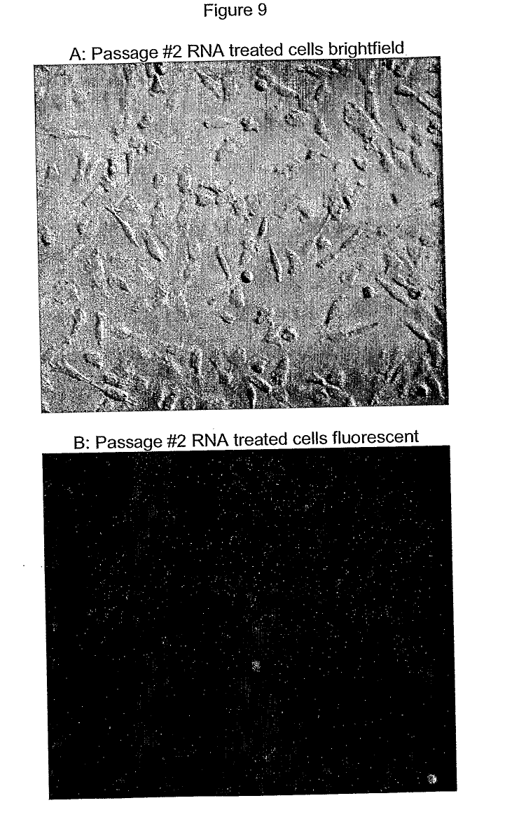

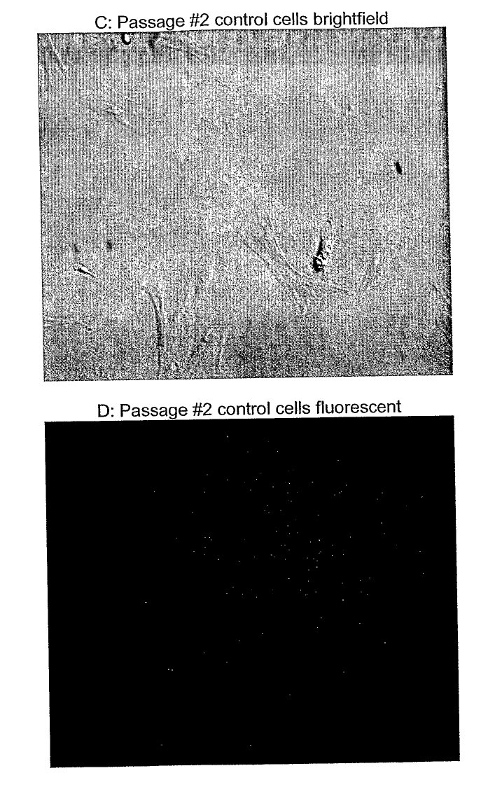

The cells were passaged once more (#2) from the 6 well plates, and the cells began to show morphological changes like C166 cells, compared to the controls (see FIGS. 9A to 9I). The cell proliferation vastly increased, and all the RNA treated cells fluoresced.

A further attribute of the C166 GFP cells is that they are resistant to G418 Genetecin selection agent. This is toxic to wild type bone marrow cells and to non-GFP C166 cells. A six well plate was set up with passaged cells of the wild type bone marrow (3 wells) and with the wild type bone marrow that had received the C166 GFP RNA (3 wells). One of the wells for each cell treatment received no G418 Genetecin, one well for each cell treatment received 0.2 mg/ml G418 Genetecin and the final wells received 1 mg/ml G418 Genetecin.

The 6 well plate was then returned to the incubator at 37° C. and 5% CO₂ for 72 hours, and was then examined.

The wells containing both the wild type bone marrow cells and the bone marrow cells which had received the C166 GFP RNA but no G418 Genetecin both showed the morphology which was expected. However, the wild type bone marrow cells when given the G418 Genetecin at both concentrations 0.2 mg/ml and 1 mg/ml killed the cells, with no adherent cells visible and just dead cells floating in the media. The C166 GFP RNA cells which received the G418 Genetecin at both concentrations 0.2 mg/ml and 1 mg/ml still retained the morphology and speed in which they colonised the wells. This shows that not only are the bone marrow cells which received the C166 GFP RNA fluorescent, colonise the wells at great speed, but are also resistant to G418 Genetecin, exactly like C166 GFP cells. The morphology and phenotypy of the cells are now those of C166 GFP cells, not wild type bone marrow cells.

In conclusion, the rat bone marrow cells with C166 GFP and C166 wild type RNA were as resistant as C166 cells were and rat bone marrow cells were not. The rat bone marrow cells with C166 GFP and C166 wild type RNA appear to now have the same morphology and phenotypy as the C166 GFP cells.

The rat bone marrow cells with C166 GFP and C166 wild type RNA were also assayed on an electrophoresis gel, and the GFP portion was in exactly the same position as in the C166 GFP cells, added to this, a sequence of the DNA from the rat bone marrow cells with C166 GFP and C166 wild type RNA was carried out, and this matched base for base the C166 GFP cells.

### Example 6

### In Vivo Stimulation of Resident Stem Cells Via Exogenous RNA Stimulated Differentiation, Migration and Integration

Given the powerful stimulatory effects of exogenous RNA on stem cells established in Examples 1 to 4, and the effects of these cells on repairing age related damage in a mammalian model, a further Example is given, establishing the effects of primary tissue derived RNA on host animal resident stem cells. To this end, neonate rats received an intraperitoneal injection of donor GFP-expressing crude bone marrow at age 1 day postnatal. Each animal received approximately 800,000 cells in a 0.2 ml injection. These foreign cells were readily integrated in host bone marrow and were observed to contribute to this biological environment. At age 90 days, GFP bone marrow grafted animals were randomly assigned to two groups.

Experimental animals received an injection of brain RNA, control animals received an injection of physiological saline. Experimental brain RNA was prepared as outlined in Example 1. Injection was conducted sub-cutaneously. Each animal received one whole brain equivalent of donor RNA in a 0.5 ml injection. Controls received an equivalent injection of a physiological saline.

The results obtained showed a significant thickening of recipient cortex (p<0.0001) in experimental animals compared to control animals. Further, a significant number of differentiated neurones and glia in experimental animals showed expression of GFP indicating infiltration of resident bone marrow stem cells into the brain following application of exogenous brain RNA.

### Example 7

### Induced Differentiation of Steal Cells Via Exogenous RNA Isolated from a Primary Cell Culture of Cortical Neurones

A purified culture of embryonic cortical neurones was established in the laboratory following the protocol of Saneto and deVellis (1987). Briefly, time mated Sprague Dawley female rats were sacrificed at day 16 of gestation. The abdominal area was sterilised with 70% alcohol and the uteri exposed. Uteri containing the embryos were then dissected free from the uteri and placed in a large 100 mm Petri dish. All the above procedures were conducted on a clean bench outside the sterile hood to prevent contamination. All further procedures were conducted under sterile conditions.

Intact uteri were then washed with physiological saline and transferred to another sterile Petri dish. Embryos were then dissected free from the uteri and placed in a new Petri dish for brain dissection. Brain tissue was exposed and gently removed with a spatula and cortices were dissected under a dissecting microscope. Meninges were then dissected clear in physiological saline. After cortices were processed, they were gently disrupted with repeated passage through a 10 ml glass pipette. The cell suspension was then passed through a Nitex 130 filter (mesh size 130 μm) and the filtrate centrifuged at 40 g. The pellet was then re-dispersed in serum free basal media (Saneto & deVellis, 1987, supra) and passed through Nitex 33 (mesh size 33 μm) and cells counted.

The suspension was supplemented with insulin (5 μg/ml) and transferrin (100 μg/ml) to form neurone-defined medium. Cells were seeded at a density of 1×10⁵ per well on 24 well culture plates pre-coated with polylysine (2.5 μg/ml). Cultures are reported as containing more than 95% neurones by immunological criteria of expressing the marker neurofilament protein, while not expressing the biochemical and immunological markers for astrocytes and oligodendrocytes (Saneto & deVellis, 1987, supra). Media was changed every third day post plating and cultures were maintained for 12 days prior to RNA extraction.

RNA was extracted from the primary cortical neurone cultures via a commercial kit (RNAzol) using the manufacturer's protocol. Resultant RNA was collected and redissolved in bone marrow culture medium (as defined in Example 1) just prior to application to a confluent colony of rat bone marrow cells prepared as in Example 1. Each recipient bone marrow culture well received the total RNA extracted from one complete 24 well primary neuronal culture (although similar results were obtained a wide variety of exogenous RNA concentrations).

Bone marrow stem cells were examined microscopically 24 hours after application of exogenous RNA dissolved in media. Control bone marrow stem cells received an equal amount of RNAzol prepared bone marrow stem cell RNA.

Results showed all experimental stem cell wells produced clearly differentiated neurones, which stained positively for neuronal markers. No observable change in stem cell differentiation was found in the Bone marrow RNA treated wells. These results suggest that donor RNA from a purified cell source may induce highly specific stem cell differentiation.

The differentiation inducing effect of exogenous RNA fractions was sensitive to pre-treating the donor RNA with RNaze yet insensitive to trypsin. This suggests that RNA mediated the effect. These effects may be repeated using a wide range of RNA doses delivered exogenously by a variety of delivery methods and vehicles including liposomes or electroporation.

### Example 8

### Specific Stem Cell Differentiation Induced by Exogenous RNA

### Marrow Harvest and Culture.

Bone marrow stromal (mesenchymal) stem cells were obtained from adult Sprague Dawley rats. The technique is based upon the protocol of Owen and Friedenstein (1988), and represents a typical established adult stem cell source suitable for expansion in vitro.

Briefly, after schedule one killing (cervical dislocation), tibia and femora were excised within 5 minutes of death. All connective and muscular tissue was removed from the bones and all further procedures were conducted under sterile conditions.

Marrow was expelled from the bones by flushing the bones with media (α-MEMS—Gibco Invitrogen Co. UK) containing 10% foetal calf serum, and 1% penicillin/streptomycin. Flushing was achieved by inserting a 25-gauge needle attached to a 5 ml plastic barrel into the neck of the bone (cut at both distal and proximal end) and expelling 2 ml of media through the bone. The media and bone marrow sample were collected in sterile universal containers. Bone marrow cells were subsequently dissociated by gentle trituration through a 19-gauge needle approximately 10 times. One ml of aspirate was then placed in six well plates (SLS Ltd. UK). Two ml of fresh α-MEMS was then added to each well giving a plating density of approximately 200,000 cells per ml. Plates were then incubated at 37° C., in 5% CO₂ in air and left undisturbed for 24 to 48 hours (Harrison & Rae, 1997).

Following this time period, marrow derived stem cells were isolated from non-plastic adherent cells by aspirating the culture media from the plate. Plastic adherent marrow stromal stem cells remained, and were supported by the addition of 2 ml of fresh α-MEMS (10% foetal calf serum and 1% penicillin/streptomycin). New media was applied every 72 hours until the plate was confluent with colony forming units (CFU's) confirmed by microscope analysis (Owen & Friedenstein, 1988, supra). Under optimal conditions this required 5 to 7 days at 37° C.

Positive control cells were primary cultures of embryonic whole brain (E18) maintained on identical 6 well plates

### Experimental Design

Plates of cells were randomly assigned to 5 groups of treatment:

Group 1: Brain RNA on BMSC 150 μg/ml
Group 2: Brain RNA+RNase 150 μg/ml
Group 3: Spleen RNA on BMSC 150 μg/ml
Group 4: No RNA added to BMSC
Group 5: Positive Control E18 brain primary culture

### RNA Methods

### RNA Extraction—Acid Guanidinium Thiocyanate-Phenol-Chloroform Method

RNA extraction was modified to further minimize DNA contamination by an additional step of DNase treatment (Ambion). Purity and concentration was confirmed by analysis on Nanodrop spectrophotometer.

Tissue Preparation.
1. Add tissue to 1 ml Solution D (@4° C.)
2. Homogenise

RNA Extraction.
1. Add 0.1 ml sodium acetate (0.2M, pH 4.0)
2. Invert to mix
3. Add 1 ml of phenol chloroform-iso-amyl alcohol (25:24:1)
4. Shake vigorously for 10 seconds
5. Cool on ice for 15 min.
6. Transfer solution to 2 ml tubes—1.2 ml in each
7. Centrifuge at 10,000 g for 20 min @4° C.

RNA Precipitation.
1. Transfer the top (aqueous) phase to a fresh tube, max of 0.5 ml.
2. Add 1 ml isopropanol
3. Incubate at −20° C. for 5 min to precipitate RNA
4. Centrifuge at 10,000 g for 10 min @4° C. RNA pellet should be seen at base of tube.
5. Discard the supernatant, air dry the pellet. Do not allow the pellet to dry out completely as this will make the pellet very difficult to resuspend.
6. Dissolve the RNA pellet in 50 μl RNase free water for 10 min.
7. Transfer immediately to ice before use, or to storage at −20° C.
8. Repellet RNA and treat with DNase

DNase Protocol
Dilute each of the RNA samples in 1000 μl nuclease free water.
Put 100 μl into a well of a 96 well plate.
Add 10 μl DNase I buffer to each well.
Add 2 μl rDNase I.
Incubate at 37° C. for 30 minutes.
Add a further 2 μA rDNase I.
Incubate at 37° C. for 30 minutes.
Transfer two of each well to a 0.5 cm³ eppendorf.
Add 20 μl DNase Inactivation Reagent.
Incubate at room temperature for 2 minutes, vortexing a few times.
Centrifuge at 10,000 g for 90 seconds and transfer the RNA to a fresh tube.

Solution D

| | | | |
|---|---|---|---|
| 2-mercaptoethanol | 0.36 | ml | |
| Add 2-mercaptoethanol | | | |
| to Solution A | | | |
| Solution A | 50 | ml | Shelf life 1 |
| | | | month at RT |

Solution A

| | | | |
|---|---|---|---|
| Guanidinium thiocyanate | 250 | g | |
| Distilled water | 293 | ml | |
| Sodium citrate (0.75M, pH 7) | 17.6 | ml | |
| Sarcosyl, 10% @65° C. | 26.4 | ml | Solution A shelf |
| | | | life 3 months at RT |

### RNase Treatment

One sample of brain RNA was treated with RNase prior to addition to the cell sample in Group 2.
Remove the ethanol from the RNA sample and air dry for 10 minutes.
Add 0.25 cm³ of the RNase (at a concentration already made up of 1 mg/cm³ in PBS). Each RNase tube will give enough RNase for 4 eppendorfs of RNA.
Incubate the RNA+RNase at 37° C. for 5 hours.
Add 0.5 cm³ Bentonite (stock solution 10 μg/cm³ in PBS).
Incubate for 1 hour at room temperature
Microcentrifuge for 10 minutes at 1500 rpm to precipitate the Bentonite.
Carefully remove the supernatant and use.

### Results & Conclusion

By day 4 post treatment there was clear morphological changes in the brain RNA treated stem cells. Cells could be clearly identified as neuronal and glial by their morphology. All RNA treated wells showed the same morphologies and distribution of these morphologies as the positive control wells (Group 5) primary cultures of brain tissue. RNase (Group 2) treatment destroyed this differentiation effect showing the active inducer of differentiation to be the RNA fraction. Specificity of the differentiation was confirmed by the spleen RNA treated group. Here, differentiation of the stem cells showed a different morphology involving aggregates of rounded cells with a spleenocyte-like morphology. Untreated BMSC retained their normal morphology throughout the experiment.

Cells were maintained in culture for 9 weeks and taken through 4 passages. Each group maintained their induced differentiation.

DNase-free naked RNA added to BMSC in culture can induce specific differentiation appropriate to the donor tissue. This transformation is stable over time and passage of the cells. The active inducer of the differentiation is RNA as the effect is destroyed by degradation of the RNA by RNase.

### Example 9

### Induction of Nerve Tissue Specific Expression of Fluorescence in BMSC by Exogenous RNA

Bone marrow stem cells were extracted from the tibias of B6.Cg-Tg(Thy-CFP) 23Jrs/J mice (Jackson Laboratory USA) and maintained in culture using the methods outlined above. These mice express a special variant of GFP (cyan-CFP) at high levels in motor and sensory neurones, as well as a sub set of central neurones. No expression is detected in non-neural cells.

Cultures were maintained in 6 well culture plates for 18 days (approx. 80% confluent) with a media change every 72 hours. Cells were observed under fluorescence microscopy to confirm no expression of Neurone specific fluorescence in BMSCs.

RNA was extracted as reported in Example 8 from C57/black wild type mouse brain (adult) and analysed for purity and concentration as reported in Example 8.

RNA was added to BMSCs at 120 μg/ml in serum free media with an exposure time of 60 minutes. After exposure to RNA cells were washed with fresh media and maintained in long term culture.

Control BMSC were exposed to serum free media for one hour with no RNA and similarly maintained.

### Results

Seventy two hours after treatment with wild type C57/black mouse brain RNA, B6.Cg-Tg(Thy-CFP) 23Jrs/J BMSCs showed some fluorescence. No fluorescence was evident in control (no brain RNA) B6.Cg-Tg(Thy-CFP) 23Jrs/J BMSCs. Cells were observed at every media change and bright fluorescence was evident in all treated wells throughout the 4 month duration of the study. At no time interval did any untreated well show any fluorescence.

Further, Brain RNA treated BMSCs showed extensive morphological changes towards a neural phenotype.

### Conclusions

Expression of cyan-CFP at high levels in differentiated BMSCs confirms that the stem cells had been induced to differentiate into neural tissues. Further, this was a very stable differentiation as the RNA induced differentiation persisted for at least 4 months in culture.

The experiment was also repeated using the same protocols using a control group which received muscle RNA which should not induce Cg-Tg(Thy-CFP) 23Jrs/J BMSCs to fluoresce. Experimental Cg-Tg(Thy-CFP) 23Jrs/J BMSCs exposed to wild type brain RNA again showed extensive and persistent fluorescence. Muscle RNA induced cells showed no evidence of fluorescence and clear muscle like morphology.

Thus, this study confirmed the initial RNA induced specific differentiation of the stem cells and showed that neural differentiation was only induced by neural derived donor RNA.

### Example 10

### Retro-Transformation of Terminally Differentiated Cells Via Exogenous Application of RNA Fractions Obtained from Stem Cell Sources

Given the powerful and specific effects of RNA tissue extracts on stem cell differentiation in Examples 1 to 6, a final example of the technology is provided. Here, the donated RNA rich extract is obtained from cultured stem cells. Its ability to reverse differentiation is tested by exogenous application to terminally differentiated adult fibroblasts to investigate if recipient mature differentiated cells could be re-differentiated to stem cell character and behaviour via stem cell derived RNA fractions. The results obtained show that stem cell type tissue may be generated from differentiated tissue.

Adult rat (Lister Hooded) fibroblasts were obtained and maintained in culture conditions according to the protocol of Kawaja et al., (1992). A biopsy of skin (approx. 1 cm²) was placed into a sterile Petri dish containing phosphate buffered saline (PBS), pH7.4. The biopsy was then dipped (×3) in another dish filled with 70% ethanol then placed back in fresh PBS and cut into 1-2 mm pieces. These explants were placed into 60-mm tissue culture dishes pre-filled with 1 ml Delbecco's minimal essential medium supplemented with 10% fetal bovine serum (FBS) and 0.1% glutamine. 10 units/ml of penicillin and 100 μg/ml streptomycin were also added. This culture was incubated with 5% CO₂ at 37° C.

After two days in such culture conditions, fibroblasts begin to migrate from the explant, at this stage an additional 2-3 ml of nutrient media was added.

When the plates reached approximately 90% confluence, they were passaged by incubating the cultures with 1-2 ml of trypsin solution and transferred to a 15-ml centrifuge tube, then centrifuged in a bench centrifuge for 10 minutes at room temperature. The supernatant was discarded and the pellet resuspended in 10 ml of culture medium. These cells were maintained in untreated 6 well plates seeded with 0.5 ml cell suspension in 2 ml of medium until confluence. At this time they could be further passaged.

Donor RNA was sourced from adult rat bone marrow mesenchymal stem cells maintained in culture as reported in Example 1 or from neural stem cells (neurospheres) cultured according to the protocol of Reynolds & Weiss (1992). All RNA rich extracts were prepared by RNAzol separation following the manufacturer protocol. Thus, two donor RNA fractions were obtained: 1) bone marrow stem cell RNA (BMS-RNA) and 2) neural stem cell RNA (NS-RNA). These fractions were dissolved respectively in fibroblast growth media at various concentrations from 0.75 μg/ml to 500 μg/ml and added to adult differentiated fibroblasts maintained in final culture wells for 5 days. Transformation of fibroblasts via stem cell derived exogenous RNA appeared across a wide range of doses.

In the results obtained, differentiated fibroblasts with no treatment of exogenous stem cell RNA showed no change in phenotype. 48 hours after RNA application, fibroblasts treated with an exogenous RNA dose of 25 μg/ml of either NS-RNA or BMS-RNA both showed a clear change in morphology. Recipient fibroblasts of NS-RNA formed floating spheres with the appearance and characteristics of neurospheres, from these neural phenotype cells began to radiate these could be easily identified as both neuronal and glial in morphology. Recipient fibroblasts of BMS-RNA, at for example 25 μg/ml, showed the classical bipolar shape of mesenchymal stem cells and were plastic adherent.

Subsequent experimentation showed these cells to be able to produce neurones and muscle tissues when further induced by exogenous RNA as described in Example 1. The retro-differentiation inducing effect of exogenous stem cell derived RNA fractions was sensitive to pre-treating the donor RNA with RNaze yet insensitive to trypsin. This suggests that the effect was mediated by RNA. These effects may be repeated using a wide range of RNA doses delivered exogenously by a variety of delivery methods and vehicles including liposomes or electroporation.

Thus, differentiated adult tissue can be retro-differentiated into stem cell like tissues when subjected to various stern cell-derived RNA fractions. The properties of the resulting cells reflect the donor stem cell morphology, behaviour and potential. Thus a novel and ethically less contentious way of obtaining both totipotent and pluripotent stem cells for a variety of applications in regenerative medicine is provided.

### Example 11

### Comparison of Spine RNA Treated Bone Marrow Stem Cells with Undifferentiated Bone Marrow Stem Cells in an Animal Model of Motor Neurone Disease

The SOD 1 mouse is a well-established animal model of human motor neurone disease. These transgenic animals begin to show hind limb paralysis at 70-90 days with aggressive loss of motor neurones and death at 120-135 days.

Thirty animals were used in the study. All were confirmed to express the SOD 1 genotype. Animals were randomly assigned into three groups as follows:
(i) group 1—bone marrow stem cells incubated with spine RNA;
(ii) group 2—bone marrow stem cells only; and

(iii) group 3—PBS injection.

Donor bone marrow stem cells were harvested and cultured as described in Example 1. Spine RNA was prepared from freshly dissected adult C57/Bl mice using the Kirby protocol described in Example 1. Stem cells to be used in group 1 were incubated with spine RNA for 5 hours (250 μg/ml), washed twice in fresh media, and then concentrated for injection at approximately 90,000 cells per animal in 0.1 ml. Stem cells prepared for injection in group 2 were maintained in culture with no exposure to RNA and given 5 hours equivalent exposure to fresh media.

Recipient animals in each group received an injection via the tail vein. Injections were mediated using a 30G needle. Injections were performed on recipient animals between the ages of 72 and 86 days at which time all animals showed hind limb paralysis. The number of animals surviving in each condition was recorded daily. Further limb movement was assessed weekly on a simple run test to observe hind and forelimb function.

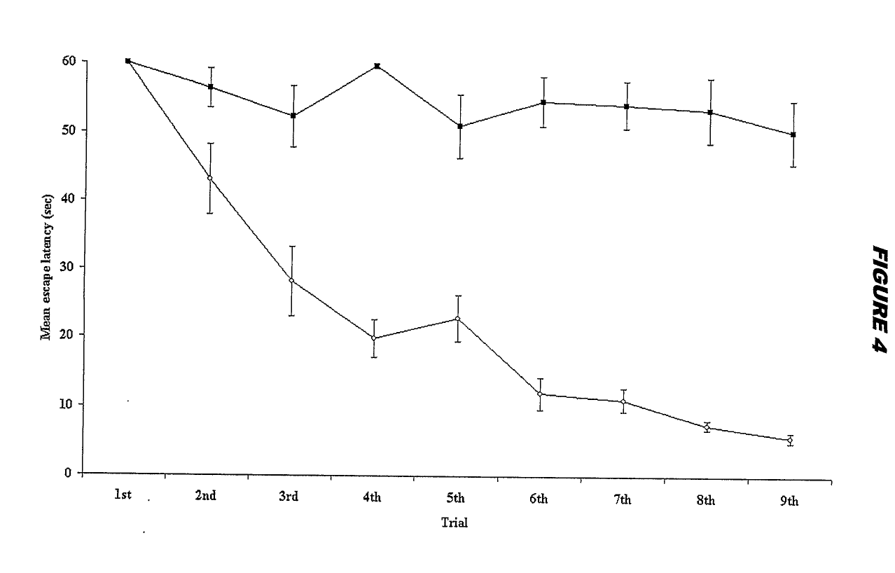

The results of this study are illustrated in FIG. 2. Pre-treatment of stem cells with spine derived RNA dramatically improved the efficacy of stem cell treatment in an established model of progressive neurodegenerative disease. Untreated bone marrow derived stem cells did have some effect but the novel step of pre-differentiating stem cells with RNA dramatically improves the effect. It is further noted from this example that all surviving animals in the RNA stem cell group (6) and the survivors in the stem cell only group (1) had complete recovery of pre-treatment paralysis and the treatment prevented further evolution of this normally progressive disease.

### Example 12

### Effects of RNA Donor Tissue Age and Developmental Stage on Stem Cell Migration Integration and Repair

Having established the effects of donor tissue derived RNA on stem cells in a variety of applications, a further example is provided investigating the effects of donor tissue developmental stage, prior to RNA extraction, on stem cell proliferation, migration and integration into host tissue.

Bone marrow stem cells were harvested and cultured as outlined in Example 1 from Tau-GFP-expressing mice. Recipient animals (N=24) were 254-299 day old C57/Bl mice randomly assigned to three recipient groups (n=8). Cultures of stem cells were randomly allocated to three conditions for RNA treatment prior to injection:
(i) group 1 foetal (E15) brain RNA+stem cells;
(ii) group 2 adult (90 day) brain RNA+stem cells; and
(iii) group 3 stem cells+no RNA.

RNA was extracted using the Kirby method as detailed in Example 1 and the appropriately sourced RNA detailed above was dissolved in media at a concentration of 200 μg/ml. Each well of recipient stem cells was incubated in 2 ml of fresh media supplement with 1 ml of RNA containing media (groups 1 & 2) or 3 ml of fresh media only (group 3) for 12 hours. Cells were then washed twice and concentrated for injection at approximately 40,000 cells in 0.3 μl of fresh media. Recipient animals were anaesthetised and cells were injected using stereotaxic guidance into the left lateral ventricle of the brain. Twenty days after surgery all groups were assessed on a mouse Morris water maze using the same training protocol as reported for rats reported in Example 2. Mice at this age show similar spatial learning deficits to old rats using this training methodology. After training, recipient rats were sacrificed and brain tissue was examined for cortical thickness and fluorescent microscopy to assess survival, proliferation and migration of GFP-expressing cells.

Behavioural results are presented in FIG. 3. Animals in both groups 1 and 2 showed excellent learning on the Morris water maze when compared to animals in group 3. This further shows the stimulatory effect of exogenous RNA treatment on stem cells in repairing age related brain damage (see Examples 2 and 8). Further, the foetal RNA+stem cell group showed significantly (p<1×10⁻¹⁰) faster acquisition of the task than the adult RNA+stem cell group. These data indicate that RNA sourced from a developmental stage when extensive neurogenesis is occurring may have a more profound effect when used to treat stem cells for tissue repair. Examining cortical thickness further supported this conclusion.

Measurement of cortex thickness in 20 identical anatomical cross sections in each animal showed a significant difference between the adult RNA+stem cells recipients and the stem cell only group (p<1×10⁻⁵), this confirms similar rat data (see Example 6). However, the cortex measures in the foetal RNA+stem cell group was also significantly thicker than the adult RNA group. Optical examination under fluorescent microscopy showed that the adult RNA+stem cell group had GFP-expressing cells extensively throughout the injected and contralateral hemispheres. However, foetal RNA+stem cell animals had approximately 30% more cells than the adult RNA group throughout the cortex of both hemispheres. GFP-expressing cells in the stem cell only group was predominantly located around the lower margins of the injected lateral ventricles and the olfactory bulbs. Only occasional cells were located in the ipsilateral cortex.

It can be concluded from this study that pre-treatment of stem cells with brain derived RNA increases their proliferation, migration and functional integration into recipient nervous systems. Further, RNA sourced from a more immature developmental stage, at an active cell generative stage, may have a more profound effect on stem cell stimulation and their consequent ameliorative effect in both age and disease related damage.

### Example 13

### A Comparison of the Stimulatory Effects of Adult Stem Cell Derived RNA on Endogenous Neural Stem Cells and their Activity

Evidence provided in Example 6 shows that exogenous RNA had a stimulatory effect on resident bone marrow stem cells in restoring age related behavioural deficits. It is also described (Example 10) that stem cell derived RNA can influence differentiated tissues. This Example investigates if direct injection of bone marrow stem cell derived RNA can stimulate endogenous repair mechanisms to ameliorate age related behavioural deficits. Various endogenous neural repair processes are now known, including direct neurogenesis mediated by neural stem cells, but also secretion of survival factors from stem cells, which may influence damaged differentiated tissues.

Bone marrow stem cells were harvested and cultured in vitro as described in Example 1. Confluent cultures were then selected for RNA extraction. RNA extraction was mediated using a commercial product RNAzol following the manufacturer's instructions. Resultant bone marrow RNA was dissolved in PBS (200 μg/15 μl) ready for injection into recipients.

Recipient Sprague Dawley rats were ex-breeder males aged between 433 days and 570 days. Due to profound age related damage to the CNS such animals cannot learn or recall the Morris water maze task. Recipients were matched for age into two groups of 10 animals:
(i) group 1—received a 15 μl injection of stem cell RNA; and
(ii) group 2—received a 15 μl injection of stem cell RNA treated with RNaze (see Example 1).

Injections were made under anaesthesia into the right lateral ventricle under stereotaxic guidance. Briefly, recipient rat was anaesthetized, head shaved and placed in a stereotaxic frame. Skin was swabbed with 100% alcohol and the skull exposed by longitudinal incision. A 1.5 mm wide hole was drilled 1.5 mm anterior to the bregma and 1.5 mm lateral to the midline. The visible dura was cut with the tip of a 30G hypodermic needle. The loaded cannula was lowered into the lateral ventricle via stereotaxic guidance and the contents ejected in 5 μl steps. The cannula was left in place for 2 minutes before removal and the incision closed with suture.

Fourteen days after injection, the aged rats were assessed blind on the Morris water maze as described in Example 2.

Results of this study are presented in FIG. 4. Control rats receiving deactivated stem cell RNA (RNaze treated) could not learn the task. There was no decrease in response latency over trials. However, the stem cell RNA treated animals all learned the task and were comparable in performance to young rats.

The stem cell derived RNA had a significant (p=1.28×10⁻⁴⁵) effect on stimulating endogenous repair mechanisms in the aged recipient brain. This may have been mediated by stimulation of resident neural stem cell neurogenesis per se or by increased production of secretory molecular products involved in tissue repair.

This experiment has also been replicated with a similar stimulatory effect using foetal (E12) derived whole brain RNA injected at a dose of 125 μg/μl (n=8) and a PBS injected control (n=8). Foetal RNA injected animals performed significantly better than control (p<1×10⁻⁵). This replication indicates that RNA prepared from developmental stages known to show increased stem cell activity may also be used to stimulate endogenous repair mechanisms.

### Example 14

### The Effects of Foetal Brain Extracted RNA on Damaged Brain Tissue In Vitro

The results of the two studies in Example 13 suggests that exogenous RNA sourced from stem cell active tissues, or stem cell derived RNA, may influence not only endogenous stem cells but may also influence resident differentiated cells. This is also shown in Example 10. The current description investigates the effects of foetal brain derived RNA on adult brain cortex cells placed in vitro.

It is well established that foetal neurones survive in tissue culture, however adult cortical neurones do not survive well. The principal reason for this is the damage suffered during initial cell preparation and plating. The trauma of dissociation is known to produce irreparable damage. It was hypothesised that RNA from an actively developing (foetal) tissue may repair such damage and enhance the survival of these cells.

RNA was extracted from 3 g of fresh foetal (E18) cortex using the Kirby protocol described in Example 1.

Adult neural tissue was cultured via the technique described in Example 7 (Saneto & deVallis, 1987). This protocol produces excellent cultures of foetal cortical neurones, however adult cortex preparations do not survive using this method. Source cortex was dissected from 48 day old Sprague Dawley rats and plated at a density of approximately 1×10⁵ into 24 well plates. 96 wells were thus prepared. 24 hours after plating, all wells were observed to have large populations of dead, necrotic and dying cells. 12 wells per 24 well plate were treated with 150 μg of foetal brain RNA dissolved in the neurone culture media. The control wells each received fresh culture media. Cells were left undisturbed for a further 48 hours then all wells received a media change with fresh media. Media changes were repeated every 3 days. Cells were observed every 24 hours.

An initial observation at 24 hours post media change showed that all control wells were dead. No viable cells remained, clumps of floating debris were observed and a dense coating of dead material was found at the bottom of all control wells. All control wells were found to have cloudy discoloured media indicative of dead cultures. Experimental wells appeared in better health but still contained some dead material. Viable cells were, however, visible.

After 72 hours (second media change) all control wells were dead (and disposed of). Experimental wells contained cell debris, which was removed with the media change, however in all wells some viable cells remained attached to the plate. Visible from many cells were small neurite outgrowths and clear neural morphology.

After 96 hours all experimental wells had flourishing neurones many with visible axon and dendrite structures. 17/48 (35%) wells showed extensive cell contact and connectivity.

After 120 hours all experimental wells contained extensive cell populations showing both neurone and glia morphology. Extensive neural networks were evident in all wells.

Cells were maintained for a further 30 days and expressed neural morphology throughout.

This Example shows a novel methodology for the culture of adult neural tissue. Furthermore, it illustrated that RNA extracted from a stem cell rich foetal tissue source has a profound rescue effect on damaged cells. This suggests a novel approach to tissue repair and regeneration via foetal or cultured stem cell RNA deliverable via a variety of methods to aged, diseased tissue or intractable wounds or trauma.

### Example 15

### The Use of Rat Embryo RNA to Enhance Stem Cell Involvement in Tissue Regeneration

Adult mammals, including human beings, have poor regenerative abilities in many tissues and organs compared to foetal stages, which often have extensive regenerative abilities. Two major factors associated with this loss of regenerative ability are scar tissue formation and loss of secretory molecules that recruit new cells to injured tissues. While many laboratories have reported the integration of injected stem cells into damaged tissues, this has been on a relatively small scale. It could be hypothesised that if the signalling mechanisms of the foetal stage could be recapitulated in the adult, this would improve the ability of stem cells to effect major regeneration of structures which show little or no repair. This would include old established injuries with associated scaring which is known to inhibit stem cell migration, integration and repair potential. The methodology used is co-injection of whole embryo RNA with stem cells. The example provided illustrated the complete regeneration of an established ear punch hole lesion in adult rats following injection of whole rat embryo RNA and bone marrow stem cells.

15-day old foetuses were dissected from the uteri of time-mated Lister Hooded rats. Foetal tissues were disrupted mechanically by a Turex homgenizer in cold PBS. RNA was extracted using the Kirby protocol described in Example 1.

Bone marrow stem cells were cultured as described in Example 1 and concentrated for injection as described in Example 12.

The injury model involved 18 male Lister Hooded rats aged between 137 and 149 days at time of injection. All rats received a 1.5 mm hole punch injury to the left ear at 30 days prior to injection date to model an old established injury. Rats at this age do not regenerate ear tissue.

Experimental animals (n=6) received a tail vein injection of 800 kg of embryo RNA dissolved in 0.3 ml of PBS. One hour later, the animals received a second injection of approximately 2×10⁵ bone marrow stem cells suspended in 0.3 ml of a-MEMS culture media. Control animals (n=6) received an initial tail vein injection of approximately 2×10⁵ bone marrow stem cells followed by a second injection of 0.3 ml PBS 1 hour later. A further group, no treatment controls (n=6), were ear clipped but received no treatment.

Animals were observed daily for any signs of regeneration of ear injury. Results showed no evidence of tissue repair or remodelling in the no treatment control group. Similarly, the stem cell only injected controls also failed to show any evidence of repair other than a slight inflammatory response lasting 17 hours in one animal around the site of the injury. The experimental animals treated with a combination of embryo RNA and stem cells showed complete closure of the injury in all animals between 6 and 9 days post injection. In 5 of the 6 experimental animals there was complete remodelling of the injury to the extent that there was no visible scar or evidence of the original lesion. Animal 3 showed complete closure of the lesion but a visible skin covered depression remained.

The results clearly show that stem cell mediated tissue repair and regeneration can be dramatically improved by co-injecting embryo derived RNA fractions with the stem cells. It is clear, from this example and other similar studies by the present inventors, that the embryo RNA alters the host tissue environment around the tissue to signal injected stem cells to the damaged area. Further, the established scarring of the injury was similarly altered to provide a permissive environment for stem cell infiltration and subsequent repair of the lesion. With such co-treatment, stem cells are recruited to the damaged tissues and can reverse the damage once in location by regeneration of the relevant tissue types. Of great significance is the fact that the damage model used in this example is an old well established injury which stem cell injection alone cannot repair. This method provides a novel method of improving the efficacy of any potential stem cell therapy. Similar results have also been found using RNA extracted from foetal tissue maintained in tissue culture and injected up to 48 hours prior to stem cell injection. Longer intervals have not yet been investigated. Similarly, simultaneous injection of the RNA with stem cells achieves a similar major regeneration of damaged tissue. It is postulated that the embryo RNA re-creates the permissive regenerative environment and signalling environment of the foetal period.

### Example 16

### Generation of RAT Embryonic Stem Cell-Like Cells from Adult Rat Bone Marrow Mesenchymal Stem Cells

While much emphasis has been placed on the plasticity of adult stem cells in many research laboratories, others consider embryonic stem cells to offer the most promise in the future of regenerative medicine. Embryonic stem cells have several practical disadvantages such as the ethics of generating embryonic stem cells, contamination of cell lines or availability of suitable cells. This example seeks to use embryonic stem cell extracted RNA to convert adult bone marrow stem cells to an embryonic stem cell-like cell.

Isolation, growth and maintenance of rat embryonic stem cells (RESCs) was carried out following the protocols of Fandrich et al. (2002) and Ruhnke et al. (2003). Briefly, RESCs were isolated from the dissociated inner cell mass of 4 to 5 day old blastocysts derived from time-mated Sprague Dawley rats. Embryonic stem cells were maintained on a feeder layer of mitomycin-treated embryonic fibroblasts. Culture media consisted of high-glucose Dulbecco's modified Eagles medium, 10% heat inactivated foetal bovine serum, 1% 200 mM L-glutamine, 1% penicillin/streptomycin solution (50 IU/50 μg), insulin (0.09 mg/ml), 1,000 U/ml LIF and 5 ml nucleoside solution (as reported in Ruhnke et al. 2003).

These cells grow in distinctive smooth round clumps and stained positive for alkaline phosphatase, a commonly used ES marker.

RNA was extracted from these RESCs via RNAzol prep following the manufacturer's instructions. Adult bone marrow stem cells were cultured as reported in Example 1 in 6 well culture plates. Each confluent well was assigned either experimental (n=12) or control (n=12). Experimental wells received 150 μg of RESC RNA in 3 ml of bone marrow culture media (see Example 1) at a routine media change. Control animals received 3 ml of bone marrow culture media. After 24 hours there was a noted change in morphology of some of the cells in the experimental wells. The colony forming units, typical of bone marrow mesenchymal stem cells appeared disrupted and large numbers of aggregated smooth round clumps of cells appeared floating in the media. Their morphology was reminiscent of the RESC cultures. No such structures appeared in the control wells. These floating aggregated structures were aspirated with the media and placed onto feeder layers in RESC media as described above and maintained in long term culture. Over 60 days they retained their floating round aggregate morphology. After 60 days in culture these cells stained positive for alkaline phosphatase, the ES marker. Control well media was also aspirated and placed in identical wells conducive to RESC culture, no aggregated floating structures emerged.

This experiment suggest a novel method for generating embryonic stem cell like cells from adult stem cells with fewer ethical issues to address.

### Example 17

### In-Vivo Injection of Muscle RNA from Exercise Tolerant Rats Induces Exercise Tolerance in Sedentary Rats

Exercise is known to be beneficial to muscle anatomy and physiology. During repeated exercise micro damage to skeletal muscle induces both stem cell activity and changes in muscle cell biology. Such changes facilitate an increased tolerance for exercise with practice.

RNA extracted from hind limb muscles from exercised rats was injected to sedentary animals to investigate the effects of such treatment on recipient animal performance during heavy exercise. The exercise task involved running on a revolving drum. Rats readily learned to stay on the apparatus by running at an appropriate speed dictated by the revolution speed of the drum. As the animal tires and stops running it falls into a plastic bin filled with shredded paper. Once running skill had been perfected, animals would happily run on the apparatus until exhaustion. After a period of initial training on the apparatus, run time was recorded as a measure of exercise tolerance.

Experimental Donor rats (n=10) were trained daily on a suitable exercise regimen as follows:

Week 1—Animals were given 5 trials per day (10 minutes) with inter-trial interval of 1 hour. The revolution speed was set at 15 mm/sec. If the animal fell, it was placed back on the drum for the full duration of the trial. All animals mastered this motor skill readily over this orientation week.
Week 2—Animals were given 5 trials per day with an increased speed of 37 mm/sec with a 1-hour inter trial interval. If an animal fell, it was immediately placed back on the apparatus. Each trial was of 15 minutes duration.
Week 3—Animals were given 1 trial per day at the same run speed but run until the first fall.
Week 4—Animals were given 1 trial per day to first fall criterion at a run speed of 97 mm/sec.

Control Donor rats (n=10) were not exposed to the exercise apparatus and remained in their home cage throughout the 4-week exercise period.

Both groups of donors were sacrificed at the end of week 4 and hind limb muscles dissected. RNA was extracted by the method outlined in Example 1. RNA was then stored in 900 μg doses ready for injection.

Recipient animals (n=20) were divided into two matched groups. All recipient animals received an orientation week of training on the apparatus as described in donor week 1 training. They received no further conditioning.

One day after last orientation trial recipient rats received 900 μg of RNA dissolved in 0.3 ml of PBS (IV) into the tail vein. Experimental recipients received exercised muscle RNA, control animals received un-exercised RNA.

One-week post injection all rats received a run trial as follows: 5 minutes gentle running at 15 mm/sec. All rats balanced and ran comfortably in this session. After five minutes balance trial, the speed was increased to 97 mm/sec and the duration to falling off/jumping off was recorded as a measure of exercise tolerance.

There was a clear difference between the two groups. Recipients of non-exercised RNA showed a mean exercise time of 3.54 minutes. Recipients of muscle RNA from exercised rats showed a mean exercise time of 6.19 minutes.

The RNA extracted from the exercised animals enhanced exercise tolerance in recipient animals compared to controls. These preliminary data suggest that RNA may transfer exercise induced muscle enhancement to naïve muscle via in vivo application. This may provide a valuable therapeutic approach to various muscle degenerative diseases or a novel method to improve muscle mass in disease, ageing or age related pathology. Further, the technique may be of value in agriculture.

### Example 18

### Effect of polyA Positive and polyA Negative RNA on the In Vitro Differentiation of Stem Cells

An effect has been seen with the addition of whole, unfractionated, RNA to cell cultures, with the result of the cells differentiating into cells of the type the RNA was extracted from. The following example illustrates that the polyA positive RNA fraction is the active fraction for cell differentiation.

Isolation of brain RNA. Sixteen P26 rats were sacrificed and their brains removed, placed in RNAlater™ (Ambion cat#7021) and stored on ice before incubation at 4° C. After 24 hours, the sample was removed from the RNAlater™ and placed in a SPEX CertiPrep 6850 Freezer Mill for milling under liquid nitrogen. The programme of sample preparation was 2 minutes pre-cooling, 1 minute milling, 1 minute cooling, 1 minute milling. The resulting powder was processed using an acid guanidinium thiocyanate-phenol-chloroform RNA extraction procedure (Chomczynski et al (1987) Analytical Chemistry 162, 156-159).

The tissue samples were then reconstituted in solution D (0.36 ml 2-mercaptoethanol in 50 μm Solution A (250 g guanidinium thiocyanate in 293 ml distilled water with 17.6 ml sodium citrate (0.75M, pH 7) and 26.4 ml sarcosyl (10%))).

The volume of solution D added was in a ratio of 1 ml: 0.2 g tissue. The resultant mixture was triturated using a 10 ml syringe with a 19 gauge needle. After five triturations, 10% of the volume of sodium acetate (2M, pH 4.0) was added and mixed by inversion. An equal volume of phenol chloroform-iso-amyl alcohol (in a ratio of 25:24:1) as the volume of solution D used was added the resultant mixture shaken vigorously for 10 seconds before cooling at −20° C. for 15 min. After this time, the solution was transferred to 2 ml tubes, with a maximum of 1.2 ml in each, before centrifugation at 10,000 g for 20 minutes at 4° C.

Following centrifugation, the upper (aqueous) phase was transferred to a fresh 2 ml tube, to a maximum of 0.5 ml, and 1 ml isopropanol added. This mixture was then incubated at −20° C. for 15 minutes to precipitate RNA, before centrifugation at 14,000 rpm for 20 minutes at 4° C. An RNA pellet was obtained at the base of the tube. The supernatant was removed and 1.5 ml 100% ethanol added to the pellet. The mixture was vortexed and incubated at −80° C. for 30 min. The mixture was then centrifuged at 12,000 g for 20 minutes at 4° C. The supernatant was again removed and the pellet washed with 1 ml 70% ethanol, vortexed and centrifuged at 12,000 g for 10 minutes at 4° C. The resultant pellet was stored at −20° C. until required.

At this stage, the purity and concentration of the RNA sample produced were ascertained using a Genequant spectrophotometer. The average purity (A260/A280) was 1.82 and the average concentration was 548.69 μg/ml.

The total RNA was then further purified by the addition of 0.1 volume 3M sodium acetate, 1 μl glycogen and 2.5 volumes of 100% ethanol and the resultant mixture incubated at −70° C. for 30 minutes before centrifugation at 12,000 g for 25 minutes at 4° C. The supernatant was removed by aspiration and the pellet centrifuged once more at 12,000 g for 5 minutes at 4° C. to remove any remaining supernatant. 1 cm³ 70% ethanol was added, the mixture vortexed, and the RNA repelleted by centrifuging at 12,000 g for 10 minutes at 4° C. The supernatant was then removed.

Some of the resultant RNA pellet was set aside for use as total RNA later. For the remaining total RNA, samples of not more than 2,000 were resuspended in 0.75 cm³ nuclease free water and vortexed. An equal volume of 2× binding solution (Poly(A) Purist™ mRNA purification kit, manufacturer's protocol) was added and mixed thoroughly. Each RNA sample was then added to a tube containing 100 mg oligo(dT) cellulose and mixed by inversion. The resultant mixture was then heated to 70° C. in a water bath for 5 minutes. After this time, the mixture was agitated gently for 60 minutes at room temperature. The oligo(dT) cellulose was pelleted by centrifuging the mixture at 3000 g for 3 minutes at room temperature.

Isolation of polyA negative RNA fraction. The resultant supernatant (which contains the polyA negative RNA) was removed by aspiration and diluted by the addition of three volumes of nuclease free water and 0.1 volumes of 3M sodium acetate. Three volumes of 100% ethanol were then added and the mixture mixed thoroughly before chilling to −70° C. for 30 minutes. The mixture was then centrifuged at 12,000 g for 20 minutes at 4° C. The supernatant was removed by aspiration and the pellet washed by vortexing in 1 ml 70% ethanol. The resultant suspension was centrifuged at 12,000 g for 10 minutes at 4° C., leaving a polyA negative RNA pellet. This was stored at −20° C. until required.

Isolation of polyA positive RNA fraction. Separately, 0.5 cm³ of Wash Solution 1 (Poly(A) Purist™ mRNA purification kit, manufacturer's protocol) was added to the oligo(dT) cellulose pellet (which contains the polyA positive RNA) and the mixture vortexed to resuspend the pellet. A spin column was placed in a 2 ml microfuge tube and the oligo(dT) cellulose suspension transferred to this column, which was then centrifuged at 3000 g for 3 minutes at room temperature. The filtrate was discarded from the microfuge tube and the spin column returned to the tube. This washing step was repeated a further time with Wash Solution 1 and a further three times with Wash Solution 2 (Poly(A) Purist™ mRNA purification kit, manufacturer's protocol).

The spin column was then placed in a fresh microfuge tube and 200 μl of warm THE RNA Storage Solution (Ambion cat#7001) (previously heated to 70° C. in a water bath) added to the oligo(dT) cellulose pellet. The mixture was vortexed to mix the two and the tube immediately centrifuged at 5,000 g for 2 minutes at room temperature. This addition of warm THE RNA Storage Solution was repeated a further two times.

The spin column was discarded and 40 μl 5M ammonium acetate, 1 μl glycogen and 1.1 ml 100% ethanol added to the filtrate. This mixture (which contains the polyA positive RNA) was then stored at −70° C. for 30 minutes.

To recover the polyA positive RNA, the mixture was centrifuged at 12,000 g for 30 minutes at 4° C. and the supernatant removed by aspiration and discarded. The remaining pellet was then washed with 70% ethanol and vortexed. Finally, a polyA positive RNA pellet was obtained by centrifuging the resultant mixture at 12,000 g for 10 minutes at 4° C. This sample was stored at −20° C. until required.

Addition of total brain RNA polyA positive brain RNA and polyA negative brain RNA to bone marrow cell cultures. Bone marrow cell cultures were cultured from 5 week-old rats, and the cultures grown in 75 cm² flasks for one month, going through one cell passage. The cells were confluent prior to addition of RNA.

The RNA samples were resuspended (after evaporation of residual ethanol) into a-MEMS media (Invitrogen cat#32571-093), supplemented with 10% foetal calf serum (Invitrogen cat#10108-165) and 3% penicillin/streptomycin (Invitrogen cat#15070-063). The media was removed from the cell culture flasks, and the total RNA sample applied directly to the cells. This was repeated with the polyA positive and polyA negative RNA samples. Furthermore, fresh a-MEMS was added as a control. The amount of RNA added was calculated to be 191.8 μg/ml.

After 24 hours, the cell culture media was changed. The cells were monitored daily for 6 days with photographs being taken. The cells were then passaged onto 6 well plates and photographed daily.

The cells treated with total RNA were not viable, with clumps of cells floating in the media. However, there was evidence of dendritic branching, and neurites (but not glia) were present.

The cells treated with polyA positive RNA showed signs of differentiation, with neurites, oligodendroglia and astroglia being present. The cells exhibited large projections and the differentiation survived passage.

In contrast, the cells treated with polyA negative RNA showed a lesser degree of differentiation than seen with either total RNA or polyA positive RNA. The differentiation included the presence of neurites, but not glia.

The control cells showed normal bone marrow cell growth. No signs of differentiation and no neurites or glia were seen.

These results show that total RNA and polyA positive RNA can induce a stable change in recipient stem cell differentiation. In contrast, polyA negative RNA can only induce a slight change in recipient stem cell differentiation, with this change being thought to result from a small amount of residual polyA positive RNA in the fraction.

### Example 19

### polyA RNA Separation Using the MACS mRNA Isolation Kit

In these experiments, the RNA sample used was from GFP rat brain, extracted using the method described in Example 4. The mRNA fraction was obtained using the Miltenyi Biotec μMACS mRNA isolation kit for Total RNA (cat#130-075-102). This method was performed as follows:

The RNA was heated for 3 minutes to 65° C., then placed directly on ice. Each RNA was diluted to 1000 μg total RNA with at least 1 volume of Lysis/Binding buffer. The final volume should be 0.5-5.0 ml. To this was added 25 μl Oligo(dT) MicroBeads per 100 μg total RNA. A MACS Column Type M was placed in the magnetic field of an appropriate MACS separator. The columns were rinsed with 250 μl Lysis/Binding buffer and the buffer was allowed to run through. The solution containing the total RNA was fed through the column matrix. The magnetically labelled mRNA is retained on the column. The column was rinsed with 1×250 μl Lysis/Binding buffer, and then 4 times with 250 μl Wash buffer. For elution of the mRNA, the column should remain in magnetic field. Apply 200 μl preheated Elution buffer and the mRNA was eluted by gravity.

The RNA was repelleted by adding 0.1 volume 3M sodium acetate, and mixed, 3 volumes 100% ethanol, and mixed, this was incubated for 90 minutes at −70° C. The solution was then centrifuged at 14,000 g for 20 minutes at 4° C.

The supernatant was removed, and washed with 1 ml 75% ethanol and vortexed. A final centrifugation at 14,000 g for 10 minutes at 4° C. where the RNA was repelleted.

There were 3 Poly A-fractions eluted.

The initial RNA concentration was 160 μg/ml, and this was used for the following treatments:

i) Poly A+ RNA

ii) Poly A-1 RNA

iii) Poly A-2 RNA

iv) Poly A-3 RNA

v) No treatment

The RNA was added to six well plates, 3 wells containing serum positive a-MEMS with the RNA, the other three, just containing serum positive a-MEMS.

The media was changed after 24 hours with fresh serum positive a-MEMS, and photography in brightfield and fluorescence was carried out

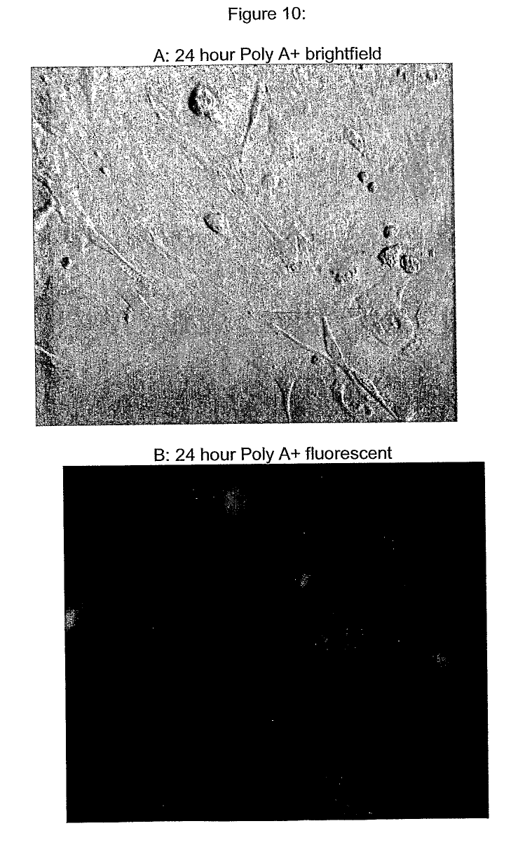

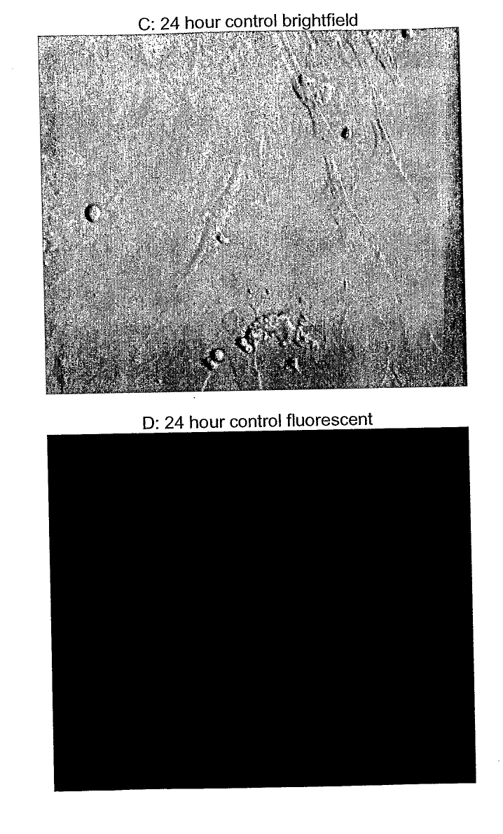

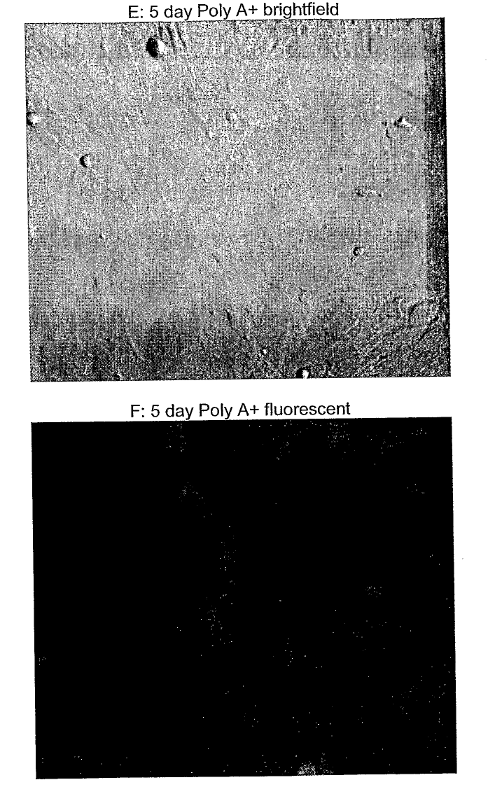

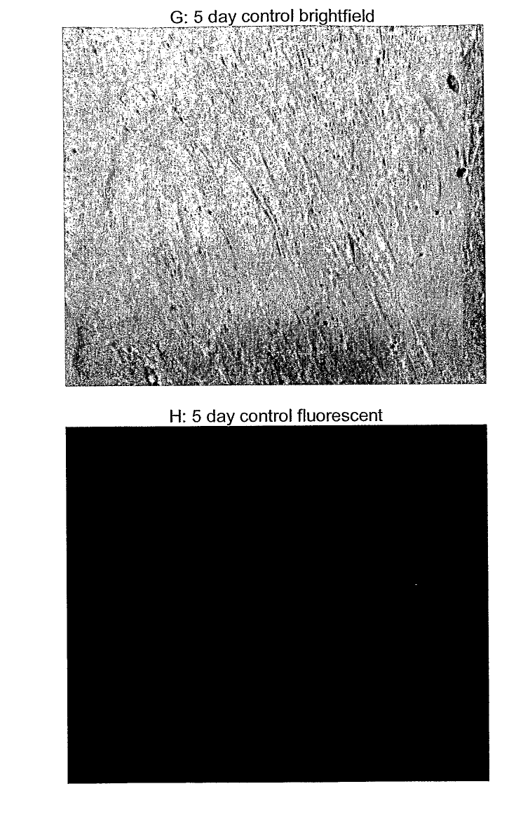

Results showed that the Poly A+ fraction did show signs of fluorescence (see FIGS. 10A to 10H). This together with the results from Example 4 (DNase/RNase treatment) suggest that it might be the RNA and specifically the Poly A+ RNA that is the active fraction that is effective to elicit the effects noted herein in which genotypic modification may be effected in a cell.

It will be understood that the invention is described above by way of example only and modifications may be made whilst remaining within the scope and spirit of the invention.

### REFERENCES

Bagnall, L. & Ray, S. (1999) Rat strain Differences on performance in the Morris water maze., Animal Technology. Vol. 50 (2). 69-77.
Dai et al. (2000) Biol Blood Marrow Transplant. 6(4). 395-407.
Fandrich, F., Lin, X., Chai, G. X. et al. (2002) Preimplantation-stage stem cells induce long term allogeneic graft acceptance without supplementary host conditioning. Nature Medicine. Vol. 8. 171-178.
Felgner et al. (1987) Proc. Nat. Acad. Aci. Vol 84. 7413-7417.
Gennaro (2000) Remington: The Science and Practice of Pharmacy. 20th edition, ISBN: 0683306472
Graham & van der Eb (1978) Virology 52:456-457.
Harrison, M A. & Rae, I F. (1997) General Techniques of Cell Culture. Cambridge Univ. Press. Cambridge.
Kawaja, et al. (1992) In Neural Transplantation: A practical Approach. IRL Press. Oxford. Keown et al. (1990) Methods in Enzymology 185:527-537
Kirby, K. S. (1956) J. Biochem. Vol. 64. 405.
Kirshenbaum et al. (1999) Curr Opin Struct Biol 9:530-5.
Mansour et al. (1988) Nature 336:348-352 (1988)
Owen, M. & Friedenstein, A J. (1988) Stromal stem cells: marrow derived osteogenic precursors CIBA Foundation Symposium 136. 42-60.
Reynolds, B. A. & Weiss, S. (1992) Science. Vol. 255. 1707-1710.
Ruhnke, M., Ungefroren, H., Zehle, G., Bader, M., Kremer, B., & Fandrich, F. (2003) Long-term culture and differentiation of rat embryonic stem cell-like cells into neuronal, glial, endothelial and hepatic lineages. Stem Cells. Vol. 21. (4).428-436.
Saneto, R. P. & de Vellis, J. (1987) Neuronal and Glial cells: cell culture of the central nervous system. In Turner, A. J. & Bachelard, H. S. (Eds.) Neurochemistry: A practical approach. IRL Press. Oxford.
Stewart, C. A. & Morris, R. G. M. (1993) The watermaze. In Sahgal. A (Ed.) Behavioural Neuroscience. A practical approach. Vol. 1. IRL Press. Oxford.
Tada, et al. (2001) Curr Biol. Vol 11(19). 1553-8.

1. A method of inducing genotypic modification in a cell, which comprises providing isolated RNA comprising RNA extractable from source tissue to the cell under conditions whereby the desired induction of genotypic modification is achieved, wherein the RNA is isolated polyA positive RNA in substantially pure form.

2. The method of claim 1, wherein the cell is modified in vitro.

3. The method of claim 1, wherein the cell is modified in vivo.

4. The method according to any one of the preceding claims, wherein the cell is a totipotent, pluripotent or unipotent stem cell of a stem cell line or derived from a tissue of an animal or plant.

5. The method according to claim 4, wherein the cell is a totipotent, pluripotent or unipotent stem cell of a human stem cell line or derived from a tissue of a human.

6. The method according to claim 4, wherein the cell undergoes differentiation into one or more desired cell types.

7. The method according to claim 1, wherein the source tissue comprises one or more cell types in common with the cell.

8. The method according to claim 1, wherein the source tissue and the cell are iso-organic.

9. The method according to claim 1, wherein the cell is dividing.

10. The method according to claim 1, wherein the cell is non-dividing.

11. The method according to claim 1, wherein the RNA consists essentially of RNA sequences that have the ability to induce one or more specific genotypic modifications in the cell.

12. The method of claim 11, wherein the RNA is obtainable by a method comprising the steps of:
i) contacting RNA extracted from source tissue with one or more nucleic acid species capable of annealing to an RNA fraction in the extract;
ii) incubating the resultant mixture under conditions whereby said one or more nucleic acid species anneal with said fraction; and
iii) isolating the annealed fraction from the remainder of the extract,
wherein said fraction comprises the RNA sequences that have the ability to induce one or more specific genotypic modifications in the cell.

13. The method according to claim 12, wherein the nucleic acid species are 17 to 25 bases long.

14. The method according to claim 13, wherein the nucleic acid species are 20 bases long.

15. The method according to any one of claims 12 to 14, wherein the nucleic acid species comprise sequence that is complementary to a sequence of DNA at the genomic region in the source tissue corresponding to the genomic region modified in the cell.

16. A cell obtained, or obtainable by the method according to claim 1.

17. A pharmaceutical composition comprising a cell of claim 16.

18. A genetically modified organism derived from a tell of claim 17.

19. (canceled)

20. The method according to claim 1, wherein said induction of genotypic modification is provided for treating a genetic disease selected from the group comprising muscular dystrophy, cystic fibrosis, haemophilia A, haemophilia B, sickle cell anaemia and cancer.

21. The method according to claim 20, wherein the cancer is selected from the group comprising melanoma, breast cancer, renal cell carcinoma and ovarian cancer.
